SEQUENCE LISTING (84 sequences)

SEQ ID NO: 1 - protein, 1368 aa, synthetic construct
  REGION 1..1368: note = Streptococcus pyogenes dCas9 (D10A, H840A)
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1053 aa, synthetic construct
  REGION 1..1053: note = Staphylococcus aureus dCas9 (Mutant: D10A and H557A)
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1053 aa, synthetic construct
  REGION 1..1053: note = Staphylococcus aureus dCas9 (Mutant: D10A and N580A)
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVHHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1053 aa, synthetic construct
  REGION 1..1053: note = Staphylococcus aureus dCas9 (Mutant: D10A and N580A)
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVHAIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 5 - protein, 984 aa, synthetic construct
  REGION 1..984: note = Campylobacter jejuni dCas9(D8A, H559A)
MARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 6 - protein, 1084 aa, synthetic construct
  REGION 1..1084: note = Corynebacterium diphtheria dCas9(D8A, H573A)
MKYHVGIAVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDAIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 7 - protein, 1107 aa, synthetic construct
  REGION 1..1107: note = Eubacterium ventriosum dCas9(D8A, H592A)
MGYTVGLAIGVASVGVAVLDENDNIVEAVSNIFDEADTSNNKVRRTLREGRRTKRRQKTRIEDFKQLWETSGYIIPHKLHLNIIELRNKGLTELLSLDELYCVLLSMLKHRGISYLEDADDGEKGNAYKKGLAFNEKQLKEKMPCEIQLERMKKYGKYHGEFIIEINDEKEYQSNVFTTKAYKKELEKIFETQRCNGNKINTKFIKKYMEIYERKREYYIGPGNEKSRTDYGIYTTRTDEEGNFIDEKNIFGKLIGKCSVYPEEYRASSASYTAQEFNLLNDLNNLKINNEKLTEFQKKEIVEIIKDASSVNMRKIIKKVIDEDIEQYSGARIDKKGKEIYHTFEIYRKLKKELKTINVDIDSFTREELDKTMDILTLNTERESIVKAFDEQKFVYEENLIKKLIEFRKNNQRLFSGWHSFSYKAMLQLIPVMYKEPKEQMQLLTEMNVFKSKKEKYVNYKYIPENEVVKEIYNPVVVKSIRTTVKILNALIKKYGYPESVVIEMPRDKNSDDEKEKIDMNQKKNQEEYEKILNKIYDEKGIEITNKDYKKQKKLVLKLKLWNEQEGLCLYSGKKIAIEDLLNHPEFFEIDAIIPKSISLDDSRSNKVLVYKTENSIKENDTPYHYLTRINGKWGFDEYKANVLELRRRGKIDDKKVNNLLCMEDITKIDVVKGFINRNLNDTRYASRVVLNEMQSFFESRKYCNTKVKVIRGSLTYQMRQDLHLKKNREESYSHHAVDAMLIAFSQKGYEAYRKIQKDCYDFETGEILDKEKWNKYIDDDEFDDILYKERMNEIRKKIIEAEEKVKYNYKIDKKCNRGLCNQTIYGTREKDGKIHKISSYNIYDDKECNSLKKMINSGKGSDLLMYNNDPKTYRDMLKILETYSSEKNPFVAYNKETGDYFRKYSKNHNGPKVEKVKYYSGQINSCIDISHKYGHAKNSKKVVLVSLNPYRTDVYYDNDTGKYYLVGVKYNHIKCVGNKYVIDSETYNELLRKEGVLNSDENLEDLNSKNITYKFSLYKNDIIQYEKGGEYYTERFLSRIKEQKNLIETKPINKPNFQRKNKKGEWENTRNQIALAKTKYVGKLVTDVLGNCYIVNMEKFSLVVDK

SEQ ID NO: 8 - protein, 1130 aa, synthetic construct
  REGION 1..1130: note = Streptococcus pasteurianus dCas9(D10A, H599A)
MTNGKILGLAIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGAKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 9 - protein, 1125 aa, synthetic construct
  REGION 1..1125: note = Lactobacillus farciminis dCas9(D10A, H611A)
MTDRISLGLAIGVASVGFSVLDIDKGKVIELGARLFNATVAAGNQDRRDMRGSRRLLNRNKQRRKDVSKLFQEYGLLDNFDRDNFHTAFNNNWNPYELRVKGLTKQLNKEELADSLYQIIKRRGISYALKDADANEDGTDYSSSLKINSQELIEKTPAQIQLQRLNDYGKVRGKVIVGDDIDDQKVLLNVFPTNAYEKEARQIIATQQQFYPEILTGEFVKEYCQILTRKRDYFVGPGNEKSRTDYGIYKTDGRTLDNLFEELIGHDKIYPDELRASAASYTAQLFNVLNDLNNLRILSYEDGKLTQIDKEKIIAEIKNNTTVVNMLNVIKKVTGCQKDDIKGFRLNEKDKPEISSMPIYRKAHKDFLKVGIDITDWPIDFIDRLSFILTLNTENGEIRKQINKRLVPDFDFLNDDLVQLIIDNKDSFDIKTNNKWHRFSLKTMNKLIPTMIERPVEQMTLLTEMGLIKKDTKRFEGNKYLPYKEIANDIFNPVASKSVREALKIVNAVLKKYGHIEYIVIEMPRDKNLDEERKQIADFQKKNKKIKDAAFNAFVKAVGSKKNVKIALSKNRKLQMGIRLWHQQQGIDPYNGEEINANDLISNPDKFEIDAIIPQSISFDDGINNKTLCFASMNQVKGQKTPYEFLNEGHGQGYAKFKAIVSKNKNFSKAKRDNYLFEENVSNIETRKRFLSRNLVDTRYSSRVVLNSLQEFFHEKSDDTKVTVIRGKFTSNMRKHWHIDKTRDTFHHHAIDASIIAATPFLRMWKKGSTIFPTKINETSIDIETGEILDDKNFDKVMYEEPYSGFISEIMNADDRIKFSHQVDKKMNRKVSDATIYSTRIGKLSKDKKDAEYVVAKVKDIYSVDGYENFKKVYKKDKTKFLLYKYDPRTFSELEKIVIDYPDKIEKVQTNGKIKAVDISPFELYRRDNGMVRKYSKKTGPAIKQLKYLDKKLGSHIDITPKNANNKHVVLQSLKPWRTDVYLNHETGEYEIMGIKYSDLKFNKNEGYGIKKDKYLKIKNIEGVSENSEFMFSLYRKDRIKVQDMETNESVELLFWSRNFSNKKYAELKPISQIDNDKVLPIYGRGRLIKRLIPKNCKIWKVNTSILGDTYYLEKESNYPQKILD

SEQ ID NO: 10 - protein, 1179 aa, synthetic construct
  REGION 1..1179: note = Sphaerochaeta globosa dCas9(D30A, H635A)
MSKKVSRRYEEQAQEICQRLGSRPYSIGLALGVGSIGVAVAAYDPIKKQPSDLVFVSSRIFIPSTGAAERRQKRGQRNSLRHRANRLKFLWKLLAERNLMLSYSEQDVPDPARLRFEDAVVRANPYELRLKGLNEQLTLSELGYALYHIANHRGSSSVRTFLDEEKSSDDKKLEEQQAMTEQLAKEKGISTFIEVLTAFNTNGLIGYRNSESVKSKGVPVPTRDIISNEIDVLLQTQKQFYQEILSDEYCDRIVSAILFENEKIVPEAGCCPYFPDEKKLPRCHFLNEERRLWEAINNARIKMPMQEGAAKRYQSASFSDEQRHILFHIARSGTDITPKLVQKEFPALKTSIIVLQGKEKAIQKIAGFRFRRLEEKSFWKRLSEEQKDDFFSAWTNTPDDKRLSKYLMKHLLLTENEVVDALKTVSLIGDYGPIGKTATQLLMKHLEDGLTYTEALERGMETGEFQELSVWEQQSLLPYYGQILTGSTQALMGKYWHSAFKEKRDSEGFFKPNTNSDEEKYGRIANPVVHQTLNELRKLMNELITILGAKPQEITVELARELKVGAEKREDIIKQQTKQEKEAVLAYSKYCEPNNLDKRYIERFRLLEDQAFVCPYCLEHISVADIAAGRADVDAIFPRDDTADNSYGNKVVAHRQCNDIKGKRTPYAAFSNTSAWGPIMHYLDETPGMWRKRRKFETNEEEYAKYLQSKGFVSRFESDNSYIAKAAKEYLRCLFNPNNVTAVGSLKGMETSILRKAWNLQGIDDLLGSRHWSKDADTSPTMRKNRDDNRHHGLDAIVALYCSRSLVQMINTMSEQGKRAVEIEAMIPIPGYASEPNLSFEAQRELFRKKILEFMDLHAFVSMKTDNDANGALLKDTVYSILGADTQGEDLVFVVKKKIKDIGVKIGDYEEVASAIRGRITDKQPKWYPMEMKDKIEQLQSKNEAALQKYKESLVQAAAVLEESNRKLIESGKKPIQLSEKTISKKALELVGGYYYLISNNKRTKTFVVKEPSNEVKGFAFDTGSNLCLDFYHDAQGKLCGEIIRKIQAMNPSYKPAYMKQGYSLYVRLYQGDVCELRASDLTEAESNLAKTTHVRLPNAKPGRTFVIIITFTEMGSGYQIYFSNLAKSKKGQDTSFTLTTIKNYDVRKVQLSSAGLVRYVSPLLVDKIEKDEVALCGE

SEQ ID NO: 11 - protein, 1168 aa, synthetic construct
  REGION 1..1168: note = Azospirillum (strain B510) dCas9(D75A, H680A)
MARPAFRAPRREHVNGWTPDPHRISKPFFILVSWHLLSRVVIDSSSGCFPGTSRDHTDKFAEWECAVQPYRLSFALGTNSIGWGLLNLDRQGKPREIRALGSRIFSDGRDPQDKASLAVARRLARQMRRRRDRYLTRRTRLMGALVRFGLMPADPAARKRLEVAVDPYLARERATRERLEPFEIGRALFHLNQRRGYKPVRTATKPDEEAGKVKEAVERLEAAIAAAGAPTLGAWFAWRKTRGETLRARLAGKGKEAAYPFYPARRMLEAEFDTLWAEQARHHPDLLTAEAREILRHRIFHQRPLKPPPVGRCTLYPDDGRAPRALPSAQRLRLFQELASLRVIHLDLSERPLTPAERDRIVAFVQGRPPKAGRKPGKVQKSVPFEKLRGLLELPPGTGFSLESDKRPELLGDETGARIAPAFGPGWTALPLEEQDALVELLLTEAEPERAIAALTARWALDEATAAKLAGATLPDFHGRYGRRAVAELLPVLERETRGDPDGRVRPIRLDEAVKLLRGGKDHSDFSREGALLDALPYYGAVLERHVAFGTGNPADPEEKRVGRVANPTVHIALNQLRHLVNAILARHGRPEEIVIELARDLKRSAEDRRREDKRQADNQKRNEERKRLILSLGERPTPRNLLKLRLWEEQGPVENRRCPYSGETISMRMLLSEQVDIDAILPFSVSLDDSAANKVVCLREANRIKRNRSPWEAFGHDSERWAGILARAEALPKNKRWRFAPDALEKLEGEGGLRARHLNDTRHLSRLAVEYLRCVCPKVRVSPGRLTALLRRRWGIDAILAEADGPPPEVPAETLDPSPAEKNRADHRHHALDAVVIGCIDRSMVQRVQLAAASAEREAAAREDNIRRVLEGFKEEPWDGFRAELERRARTIVVSHRPEHGIGGALHKETAYGPVDPPEEGFNLVVRKPIDGLSKDEINSVRDPRLRRALIDRLAIRRRDANDPATALAKAAEDLAAQPASRGIRRVRVLKKESNPIRVEHGGNPSGPRSGGPFHKLLLAGEVHHVDVALRADGRRWVGHWVTLFEAHGGRGADGAAAPPRLGDGERFLMRLHKGDCLKLEHKGRVRVMQVVKLEPSSNSVVVVEPHQVKTDRSKHVKISCDQLRARGARRVTVDPLGRVRVHAPGARVGIGGDAGRTAMEPAEDIS

SEQ ID NO: 12 - protein, 1003 aa, synthetic construct
  REGION 1..1003: note = Gluconacetobacter diazotrophicus dCas9(D11A, H587A)
MIDESLTFGIALGIGSCGWAVLRRPSAFGRKGVIEGMGSWCFDVPETSKERTPTNQIRRSNRLLRRVIRRRRNRMAAIRRLLHAAGLLPSTDSDALKRPGHDPWELRARGLDKPLKPVEFAVVLGHIAKRRGFKSAAKRKATNISSDDKKMLTALEATRERLGRYRTVGEMFARDPDFASRRRNREGKYDRTTARDDLEHEVHALFAAQRRLGQGFASPELEEAFTASAFHQRPMQDSERLVGFCPFERTEKRAAKLTPSFERFRLLARLLNLRITTPDGERPLTVDEIALVTRDLGKTAKLSIKRVRTLIGLEDNQRFTTIRPEDEDRDIVARTGGAMTGTATLRKALGEALWTDMQERPEQLDAIVQVLSFFEANETITEKLREIGLTLAVLDVLLTALDAGVFAKFKGAAHISTKAARNLLPHLEQGRRYDEACTMAGYDHAASRLSHHGQIVAKTQFNALVTEIGESIANPIARKALIEGLKQIWAMRNHWGLPGSIHVELARDVGNSIEKRREIEKHIEKNTALRARERREVHDLLDLEDVNGDTLLRYRLWKEQGGKCLYTGKAIHIRQIAATDNSVQVDAILPWSRFGDDSFNNKTLCLASANQQKKRSTPYEWLSGQTGDAWNAFVQRIETNKELRGFKKRNYLLKNAKEAEEKFRSRNLNDTRYAARLFAEAVKLLYAFGERQEKGGNRRVFTRPGALTAALRQAWGVESLKKQDGKRINDDRHHALDALTVAAVDEAEIQRLTKSFHEWEQQGLGRPLRRVEPPWESFRADVEATYPEVFVARPERRRARGEGHAATIRQVKERECTPIVFERKAVSSLKEADLERIKDGERNEAIVEAIRSWIATGRPADAPPRSPRGDIITKIRLATTIKAAVPVRGGTAGRGEMVRADVFSKPNRRGKDEWYLVPVYPHQIMNRKAWPKPPMRSIVANKDEDEWTEVGPEHQFRFSLYPRSNIEIIRPSGEVIEGYFVGLHRNTGALIPTPVGPDSYVIA

SEQ ID NO: 13 - protein, 1082 aa, synthetic construct
  REGION 1..1082: note = Neisseria cinerea dCas9(D16A, H588A)
MAAFKPNPMNYILGLAIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDAALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 14 - protein, 1140 aa, synthetic construct
  REGION 1..1140: note = Roseburia intestinalis dCas9(D24A, H628A)
MRENGSDERRRNMDEKMDYRIGLAIGIASVGWAVLQNNSDDEPVRIVDLGVRIFDTAEIPKTGESLAGPRRAARTTRRRLRRRKHRLDRIKWLFENQGLINIDDFLKRYNMAGLPDVYQLRYEALDRKLTDEELAQVLLHIAKHRGFRSTRKAETAAKENGAVLKATDENQKRMQEKGYRTVGEMIYLDEAFRTGCSWSEKGYILTPRNKAENYQHTMLRAMLVEEVKEIFSSQRRLGNEKATEELEEKYLEIMTSQRSFDLGPGMQPDGKPSPYAMEGFSDRVGKCTFLGDQGELRGAKGTYTAEYFVALQKINHTKLVNQDGETRNFTEEERRALTLLLFTQKEVKYAAVRKKLGLPEDILFYNLNYKKAATKEEQQKENQNTEKAKFIGMPYYHDYKKCLEERVKYLTENEVRDLFDEIGMILTCYKNDDSRTERLAKLGLVPIEMEGLLAYTPTKFQHLSMKAMRNIIPFLEKGMTYDKACEEAGYDFKADSKGTKQKLLTGENVNQTINEITNPVVKRSVSQTVKVINAIIRTYGSPQAINIELAREMSKTFEERRKIKGDMEKRQKNNEDVKKQIQELGKLSPTGQDILKYRLWQEQQGICMYSGKTIPLEELFKPGYDIDAILPYSITFDDSFRNKVLVTSQENRQKGNRTPYEYMGNDEQRWNEFETRVKTTIRDYKKQQKLLKKHFSEEERSEFKERNLTDTKYITTVIYNMIRQNLEMAPLNRPEKKKQVRAVNGAITAYLRKRWGLPQKNRETDTHHAMDAVVIACCTDGMIQKISRYTKVRERCYSKGTEFVDAETGEIFRPEDYSRAEWDEIFGVHIPKPWETFRAELDVRMGDDPKGFLDTHSDVALELDYPEYIYENLRPIFVSRMPNHKVTGAAHADTIRSPRHFKDEGIVLTKTALTDLKLDKDGEIDGYYNPQSDLLLYEALKKQLLLYGNDAKKAFAQDFHKPKADGTEGPVVRKVKIQKKQTMGVFVDSGNGIAENGGMVRIDVFRVNGKYYFVPVYTADVVKKVLPNRASTAHKPYGEWKVMEDKDFLFSLYSRDLIHIKSKKDIPIKMVNGGMEGIKETYAYYIGADISAANIQGIAHDSRYKFRGLGIQSLDVLEKCQIDVLGHVSVVRSEKRMGFS

SEQ ID NO: 15 - protein, 1037 aa, synthetic construct
  REGION 1..1037: note = Parvibaculum lavamentivorans dCas9(D8A, H601A)
MERIFGFAIGTTSIGFSVIDYSSTQSAGNIQRLGVRIFPEARDPDGTPLNQQRRQKRMMRRQLRRRRIRRKALNETLHEAGFLPAYGSADWPVVMADEPYELRRRGLEEGLSAYEFGRAIYHLAQHRHFKGRELEESDTPDPDVDDEKEAANERAATLKALKNEQTTLGAWLARRPPSDRKRGIHAHRNVVAEEFERLWEVQSKFHPALKSEEMRARISDTIFAQRPVFWRKNTLGECRFMPGEPLCPKGSWLSQQRRMLEKLNNLAIAGGNARPLDAEERDAILSKLQQQASMSWPGVRSALKALYKQRGEPGAEKSLKFNLELGGESKLLGNALEAKLADMFGPDWPAHPRKQEIRHAVHERLWAADYGETPDKKRVIILSEKDRKAHREAAANSFVADFGITGEQAAQLQALKLPTGWEPYSIPALNLFLAELEKGERFGALVNGPDWEGWRRTNFPHRNQPTGEILDKLPSPASKEERERISQLRNPTVVRTQNELRKVVNNLIGLYGKPDRIRIEVGRDVGKSKREREEIQSGIRRNEKQRKKATEDLIKNGIANPSRDDVEKWILWKEGQERCPYTGDQIGFNALFREGRYEVEAIWPRSRSFDNSPRNKTLCRKDVNIEKGNRMPFEAFGHDEDRWSAIQIRLQGMVSAKGGTGMSPGKVKRFLAKTMPEDFAARQLNDTRYAAKQILAQLKRLWPDMGPEAPVKVEAVTGQVTAQLRKLWTLNNILADDGEKTRADHRHHAIDALTVACTHPGMTNKLSRYWQLRDDPRAEKPALTPPWDTIRADAEKAVSEIVVSHRVRKKVSGPLHKETTYGDTGTDIKTKSGTYRQFVTRKKIESLSKGELDEIRDPRIKEIVAAHVAGRGGDPKKAFPPYPCVSPGGPEIRKVRLTSKQQLNLMAQTGNGYADLGSNHHIAIYRLPDGKADFEIVSLFDASRRLAQRNPIVQRTRADGASFVMSLAAGEAIMIPEGSKKGIWIVQGVWASGQVVLERDTDADHSTTTRPMPNPILKDDAKKVSIDPIGRVRPSND

SEQ ID NO: 16 - protein, 1132 aa, synthetic construct
  REGION 1..1132: note = Nitratifractor salsuginis (strain DSM 16511) dCas9(D8A, H611A)
MKKILGVALGITSFGYAILQETGKDLYRCLDNSVVMRNNPYDEKSGESSQSIRSTQKSMRRLIEKRKKRIRCVAQTMERYGILDYSETMKINDPKNNPIKNRWQLRAVDAWKRPLSPQELFAIFAHMAKHRGYKSIATEDLIYELELELGLNDPEKESEKKADERRQVYNALRHLEELRKKYGGETIAQTIHRAVEAGDLRSYRNHDDYEKMIRREDIEEEIEKVLLRQAELGALGLPEEQVSELIDELKACITDQEMPTIDESLFGKCTFYKDELAAPAYSYLYDLYRLYKKLADLNIDGYEVTQEDREKVIEWVEKKIAQGKNLKKITHKDLRKILGLAPEQKIFGVEDERIVKGKKEPRTFVPFFFLADIAKFKELFASIQKHPDALQIFRELAEILQRSKTPQEALDRLRALMAGKGIDTDDRELLELFKNKRSGTRELSHRYILEALPLFLEGYDEKEVQRILGFDDREDYSRYPKSLRHLHLREGNLFEKEENPINNHAVKSLASWALGLIADLSWRYGPFDEIILETTRDALPEKIRKEIDKAMREREKALDKIIGKYKKEFPSIDKRLARKIQLWERQKGLDLYSGKVINLSQLLDGSADIEAIVPQSLGGLSTDYNTIVTLKSVNAAKGNRLPGDWLAGNPDYRERIGMLSEKGLIDWKKRKNLLAQSLDEIYTENTHSKGIRATSYLEALVAQVLKRYYPFPDPELRKNGIGVRMIPGKVTSKTRSLLGIKSKSRETNFHHAEDALILSTLTRGWQNRLHRMLRDNYGKSEAELKELWKKYMPHIEGLTLADYIDEAFRRFMSKGEESLFYRDMFDTIRSISYWVDKKPLSASSHKETVYSSRHEVPTLRKNILEAFDSLNVIKDRHKLTTEEFMKRYDKEIRQKLWLHRIGNTNDESYRAVEERATQIAQILTRYQLMDAQNDKEIDEKFQQALKELITSPIEVTGKLLRKMRFVYDKLNAMQIDRGLVETDKNMLGIHISKGPNEKLIFRRMDVNNAHELQKERSGILCYLNEMLFIFNKKGLIHYGCLRSYLEKGQGSKYIALFNPRFPANPKAQPSKFTSDSKIKQVGIGSATGIIKAHLDLDGHVRSYEVFGTLPEGSIEWFKEESGYGRVEDDPHH

SEQ ID NO: 17 - protein, 997 aa, synthetic construct
  REGION 1..997: note = Campylobacter lari dCas9(D7A, H567A)
MKILGFAIGINSIGWAFVENDELKDCGVRIFTEAENPSNKESLALPRRNARSSRRRLKRRKARLVAIKRILAKELKLNFKDYVAADGELPKAYEGKLTSIYELRYKALIQQLETKDLARVILHIAKHRGYMNKNEKKSNDTKKGKILSALKNNALKLENYQSVGEYFYKEFFQKYKENTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEILNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYYEACEITNLKPKTVDEKEDFLPAFCDSIFAHELSNPVVNRAISEYRKVLNALLKKYGKVHKIHLELARDVGLSKKAREKIEDQQKNNKAINDWALKECENIGIKASAKNILKLKLWKEQKEICIYSGNKISIEHLKDEKALEVDAIYPYSRSFDDSFINKVLVFTKENQEKLNKTPFEAFGKNIEKWSKIQTLAQNLPYKKKNKILDENFKDKQQQDFISRNLNDTRYITTLIAKYTKEYLNFLPLSENENTNLKSGEKGSKIHIQTISGMLTSVLRHTWGFDKKDRNNHLHHALDAIIVAYSTSSIIKAFSDFKKNQELLKARFYAKELTSDNYKHQVKFFEPFKGFREKILSKIDEIFVSTPPRKRARGALHEETFYSEDKMIKKYNSKEGLQIALSCGRVRKIGTKYVENDTMVRVDIFKKQNKFYAIPIYVMDFALGILPNKIVITGKDKNNNPKQWQTIDESYEFCFSLYKNDLILLQKKNMQEPEFAYYNSFDIDRSRIKIKKHDNKFENLTSNQKLLFTNKDKGKNRTGIQNLKIFEKYIVTPLGDKIKADFQPRENISLKTSKKHGL

SEQ ID NO: 18 - protein, 1388 aa, synthetic construct
  REGION 1..1388: note = Streptococcus thermophilus (strain LMD-9) dCas9(D10A, H847A)
MTKPYSIGLAIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDAIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 19 - protein, 27 aa, synthetic construct
  REGION 1..27: note = linker
SSGNSNANSRGPSFSSGLVPLSLRGSH

SEQ ID NO: 20 - protein, 80 aa, synthetic construct
  REGION 1..80: note = XTEN80 linker
GGPSSGAPPPSGGSPAGSPTSTEEGTSESATPESGPGTSTEPSEGSAPGSPAGSPTSTEEGTSTEPSEGSAPGTSTEPSE

SEQ ID NO: 21 - protein, 16 aa, synthetic construct
  REGION 1..16: note = XTEN16 linker
SGSETPGTSESATPES

SEQ ID NO: 22 - protein, 62 aa, synthetic construct
  REGION 1..62: note = Exemplary KRAB domain
RTLVTFKDVFVDFTREEWKLLDTAQQIVYRNVMLENYKNLVSLGYQLTKPDVILRLEKGEEP

SEQ ID NO: 23 - protein, 302 aa, synthetic construct
  REGION 1..302: note = DNMT3A
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 24 - protein, 215 aa, synthetic construct
  REGION 1..215: note = DNMT3L
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 25 - protein, 16 aa, synthetic construct
  REGION 1..16: note = exemplary nuclear localization sequence1
APKKKRKVGIHGVPAA

SEQ ID NO: 26 - protein, 16 aa, synthetic construct
  REGION 1..16: note = exemplary nuclear localization sequence2
KRPAATKKAGQAKKKK

SEQ ID NO: 27 - protein, 544 aa, synthetic construct
  REGION 1..544: note = DNMT3A-DNMT3L fusion peptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 28 - protein, 2111 aa, synthetic construct
  REGION 1..2111: note = DNMT3A-DNMT3L(3A3L)-dCas9-KRAB
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGSGSETPGTSESATPESRTLVTFKDVFVDFTREEWKLLDTAQQIVYRNVMLENYKNLVSLGYQLTKPDVILRLEKGEEP

SEQ ID NO: 29 - RNA, 20 nt, synthetic construct
  modified_base 1: mod_base = OTHER; note = thymine
  modified_base 6: mod_base = OTHER; note = thymine
  modified_base 16: mod_base = OTHER; note = thymine
  misc_feature 1..20: note = mouse VEGFA sgRNA1
  source 1..20: mol_type = other RNA
tcagctcgcccccagtgccg

SEQ ID NO: 30 - RNA, 20 nt, synthetic construct
  modified_base 19: mod_base = OTHER; note = thymine
  misc_feature 1..20: note = mouse VEGFA sgRNA2
  source 1..20: mol_type = other RNA
gcagcgaggccgcggcactg

SEQ ID NO: 31 - RNA, 20 nt, synthetic construct
  modified_base 14: mod_base = OTHER; note = thymine
  misc_feature 1..20: note = mouse VEGFA sgRNA3
  source 1..20: mol_type = other RNA
ggggcagccgagctgcagcg

SEQ ID NO: 32 - RNA, 20 nt, synthetic construct
  modified_base 3: mod_base = OTHER; note = thymine
  modified_base 14: mod_base = OTHER; note = thymine
  misc_feature 1..20: note = mouse VEGFA sgRNA4
  source 1..20: mol_type = other RNA
actgggggcgagctgagcgg

SEQ ID NO: 33 - RNA, 20 nt, synthetic construct
  modified_base 18: mod_base = OTHER; note = thymine
  misc_feature 1..20: note = mouse VEGFA sgRNA5
  source 1..20: mol_type = other RNA
cagcgaggccgcggcactgg

SEQ ID NO: 34 - RNA, 20 nt, synthetic construct
  modified_base 8: mod_base = OTHER; note = thymine
  misc_feature 1..20: note = mouse VEGFA sgRNA6
  source 1..20: mol_type = other RNA
gccgagctgcagcgaggccg

SEQ ID NO: 35 - RNA, 20 nt, synthetic construct
  modified_base 10: mod_base = OTHER; note = thymine
  modified_base 14: mod_base = OTHER; note = thymine
  modified_base 20: mod_base = OTHER; note = thymine
  misc_feature 1..20: note = mouse VEGFA sgRNA7
  source 1..20: mol_type = other RNA
gccgcggcctcgctgcagct

SEQ ID NO: 36 - RNA, 20 nt, synthetic construct
  modified_base 12: mod_base = OTHER; note = thymine
  modified_base 14: mod_base = OTHER; note = thymine
  modified_base 16: mod_base = OTHER; note = thymine
  misc_feature 1..20: note = human VEGFA sgRNA1
  source 1..20: mol_type = other RNA
agcaccagcgctctgtcggg

SEQ ID NO: 37 - RNA, 20 nt, synthetic construct
  modified_base 13: mod_base = OTHER; note = thymine
  modified_base 17: mod_base = OTHER; note = thymine
  misc_feature 1..20: note = human VEGFA sgRNA2
  source 1..20: mol_type = other RNA
ggggcagccgggtagctcgg

SEQ ID NO: 38 - RNA, 20 nt, synthetic construct
  modified_base 4: mod_base = OTHER; note = thymine
  modified_base 16: mod_base = OTHER; note = thymine
  modified_base 18: mod_base = OTHER; note = thymine
  modified_base 20: mod_base = OTHER; note = thymine
  misc_feature 1..20: note = human VEGFA sgRNA3
  source 1..20: mol_type = other RNA
ggctagcaccagcgctctgt

SEQ ID NO: 39 - RNA, 20 nt, synthetic construct
  modified_base 3: mod_base = OTHER; note = thymine
  modified_base 15: mod_base = OTHER; note = thymine
  modified_base 17: mod_base = OTHER; note = thymine
  modified_base 19: mod_base = OTHER; note = thymine
  misc_feature 1..20: note = human VEGFA sgRNA4
  source 1..20: mol_type = other RNA
gctagcaccagcgctctgtc

SEQ ID NO: 40 - RNA, 20 nt, synthetic construct
  modified_base 7: mod_base = OTHER; note = thymine
  modified_base 11: mod_base = OTHER; note = thymine
  modified_base 18: mod_base = OTHER; note = thymine
  misc_feature 1..20: note = human VEGFA sgRNA5
  source 1..20: mol_type = other RNA
gccgggtagctcggaggtcg

SEQ ID NO: 41 - RNA, 20 nt, synthetic construct
  modified_base 3: mod_base = OTHER; note = thymine
  modified_base 10: mod_base = OTHER; note = thymine
  modified_base 13: mod_base = OTHER; note = thymine
  modified_base 19: mod_base = OTHER; note = thymine
  misc_feature 1..20: note = human VEGFA sgRNA6
  source 1..20: mol_type = other RNA
gctcggaggtcgtggcgctg

SEQ ID NO: 42 - RNA, 20 nt, synthetic construct
  modified_base 5: mod_base = OTHER; note = thymine
  modified_base 7: mod_base = OTHER; note = thymine
  modified_base 9: mod_base = OTHER; note = thymine
  misc_feature 1..20: note = human VEGFA sgRNA7
  source 1..20: mol_type = other RNA
gcgctctgtcgggaggcgca

SEQ ID NO: 43 - RNA, 20 nt, synthetic construct
  modified_base 4: mod_base = OTHER; note = thymine
  modified_base 11: mod_base = OTHER; note = thymine
  modified_base 14: mod_base = OTHER; note = thymine
  modified_base 20: mod_base = OTHER; note = thymine
  misc_feature 1..20: note = human VEGFA sgRNA8
  source 1..20: mol_type = other RNA
agctcggaggtcgtggcgct

SEQ ID NO: 44 - RNA, 20 nt, synthetic construct
  modified_base 4: mod_base = OTHER; note = thymine
  modified_base 6: mod_base = OTHER; note = thymine
  modified_base 8: mod_base = OTHER; note = thymine
  misc_feature 1..20: note = human VEGFA sgRNA9
  source 1..20: mol_type = other RNA
cgctctgtcgggaggcgcag

SEQ ID NO: 45 - RNA, 20 nt, synthetic construct
  modified_base 2: mod_base = OTHER; note = thymine
  modified_base 9: mod_base = OTHER; note = thymine
  modified_base 12: mod_base = OTHER; note = thymine
  modified_base 18: mod_base = OTHER; note = thymine
  misc_feature 1..20: note = human VEGFA sgRNA10
  source 1..20: mol_type = other RNA
ctcggaggtcgtggcgctgg

SEQ ID NO: 46 - RNA, 20 nt, synthetic construct
  modified_base 1: mod_base = OTHER; note = thymine
  modified_base 5: mod_base = OTHER; note = thymine
  modified_base 12: mod_base = OTHER; note = thymine
  modified_base 15: mod_base = OTHER; note = thymine
  misc_feature 1..20: note = human VEGFA sgRNA11
  source 1..20: mol_type = other RNA
tagctcggaggtcgtggcgc

SEQ ID NO: 47 - RNA, 20 nt, synthetic construct
  modified_base 10: mod_base = OTHER; note = thymine
  modified_base 17: mod_base = OTHER; note = thymine
  misc_feature 1..20: note = human VEGFA sgRNA12
  source 1..20: mol_type = other RNA
gccacgacctccgagctacc

SEQ ID NO: 48 - RNA, 20 nt, synthetic construct
  modified_base 4..5: mod_base = OTHER; note = thymine
  modified_base 9: mod_base = OTHER; note = thymine
  modified_base 17: mod_base = OTHER; note = thymine
  misc_feature 1..20: note = human VEGFA sgRNA13
  source 1..20: mol_type = other RNA
cggttaggtggaccggtcag

SEQ ID NO: 49 - RNA, 20 nt, synthetic construct
  modified_base 9: mod_base = OTHER; note = thymine
  modified_base 13: mod_base = OTHER; note = thymine
  modified_base 16..19: mod_base = OTHER; note = thymine
  misc_feature 1..20: note = human VEGFA sgRNA14
  source 1..20: mol_type = other RNA
ggggcggatgggtaattttc

SEQ ID NO: 50 - RNA, 20 nt, synthetic construct
  modified_base 8: mod_base = OTHER; note = thymine
  misc_feature 1..20: note = human VEGFA sgRNA15
  source 1..20: mol_type = other RNA
gggaagctcgacccccacca

SEQ ID NO: 51 - RNA, 20 nt, synthetic construct
  modified_base 2: mod_base = OTHER; note = thymine
  modified_base 8..9: mod_base = OTHER; note = thymine
  modified_base 14..15: mod_base = OTHER; note = thymine
  modified_base 18..19: mod_base = OTHER; note = thymine
  misc_feature 1..20: note = human VEGFA sgRNA16
  source 1..20: mol_type = other RNA
gtcgagcttccccttcattg

SEQ ID NO: 52 - RNA, 20 nt, synthetic construct
  modified_base 5..6: mod_base = OTHER; note = thymine
  modified_base 11..12: mod_base = OTHER; note = thymine
  modified_base 15..16: mod_base = OTHER; note = thymine
  misc_feature 1..20: note = human VEGFA sgRNA17
  source 1..20: mol_type = other RNA
gagcttccccttcattgcgg

SEQ ID NO: 53 - RNA, 20 nt, synthetic construct
  modified_base 19: mod_base = OTHER; note = thymine
  misc_feature 1..20: note = human VEGFA sgRNA18
  source 1..20: mol_type = other RNA
gcccgggcccgagccgcgtg

SEQ ID NO: 54 - RNA, 20 nt, synthetic construct
  modified_base 3: mod_base = OTHER; note = thymine
  modified_base 6: mod_base = OTHER; note = thymine
  modified_base 18: mod_base = OTHER; note = thymine
  misc_feature 1..20: note = human VEGFA sgRNA19
  source 1..20: mol_type = other RNA
gctggtagcggggaggatcg

SEQ ID NO: 55 - RNA, 20 nt, synthetic construct
  modified_base 3: mod_base = OTHER; note = thymine
  modified_base 15: mod_base = OTHER; note = thymine
  misc_feature 1..20: note = human VEGFA sgRNA20
  source 1..20: mol_type = other RNA
ggtagcggggaggatcgcgg

SEQ ID NO: 56 - RNA, 20 nt, synthetic construct
  modified_base 9: mod_base = OTHER; note = thymine
  modified_base 19..20: mod_base = OTHER; note = thymine
  misc_feature 1..20: note = human VEGFA sgRNA21
  source 1..20: mol_type = other RNA
ggggaggatcgcggaggctt

SEQ ID NO: 57 - RNA, 20 nt, synthetic construct
  modified_base 8: mod_base = OTHER; note = thymine
  modified_base 18..19: mod_base = OTHER; note = thymine
  misc_feature 1..20: note = human VEGFA sgRNA22
  source 1..20: mol_type = other RNA
gggaggatcgcggaggcttg

SEQ ID NO: 58 - RNA, 20 nt, synthetic construct
  modified_base 8: mod_base = OTHER; note = thymine
  modified_base 17: mod_base = OTHER; note = thymine
  misc_feature 1..20: note = human VEGFA sgRNA23
  source 1..20: mol_type = other RNA
ggaccggtcagcggactcac

SEQ ID NO: 59 - RNA, 76 nt, synthetic construct
  modified_base 2..5: mod_base = OTHER; note = thymine
  modified_base 11: mod_base = OTHER; note = thymine
  modified_base 17: mod_base = OTHER; note = thymine
  modified_base 24..25: mod_base = OTHER; note = thymine
  modified_base 30: mod_base = OTHER; note = thymine
  modified_base 36: mod_base = OTHER; note = thymine
  modified_base 39: mod_base = OTHER; note = thymine
  modified_base 43..44: mod_base = OTHER; note = thymine
  modified_base 46: mod_base = OTHER; note = thymine
  modified_base 51..52: mod_base = OTHER; note = thymine
  modified_base 60: mod_base = OTHER; note = thymine
  modified_base 70: mod_base = OTHER; note = thymine
  modified_base 74: mod_base = OTHER; note = thymine
  misc_feature 1..76: note = Exemplary tracr sequence
  source 1..76: mol_type = other RNA
gttttagagctagaaatagcaagttaaaataaggctagtccgttatcaacttgaaaaagtggcaccgagtcggtgc

SEQ ID NO: 60 - RNA, 20 nt, synthetic construct
  modified_base 1: mod_base = OTHER; note = thymine
  modified_base 9: mod_base = OTHER; note = thymine
  modified_base 14: mod_base = OTHER; note = thymine
  misc_feature 1..20: note = Rabbit VEGFA sgRNA1
  source 1..20: mol_type = other RNA
tggaaccctggagtgacccc

SEQ ID NO: 61 - RNA, 20 nt, synthetic construct
  modified_base 10: mod_base = OTHER; note = thymine
  modified_base 12..13: mod_base = OTHER; note = thymine
  misc_feature 1..20: note = Rabbit VEGFA sgRNA2
  source 1..20: mol_type = other RNA
gcccagagctgttggaaccc

SEQ ID NO: 62 - RNA, 20 nt, synthetic construct
  modified_base 13: mod_base = OTHER; note = thymine
  modified_base 17: mod_base = OTHER; note = thymine
  misc_feature 1..20: note = Rabbit VEGFA sgRNA3
  source 1..20: mol_type = other RNA
ggaggccgggggtcactcca

SEQ ID NO: 63 - RNA, 20 nt, synthetic construct
  modified_base 9: mod_base = OTHER; note = thymine
  modified_base 20: mod_base = OTHER; note = thymine
  misc_feature 1..20: note = Rabbit VEGFA sgRNA4
  source 1..20: mol_type = other RNA
ggagcccgtcagggacgggt

SEQ ID NO: 64 - RNA, 20 nt, synthetic construct
  modified_base 1: mod_base = OTHER; note = thymine
  modified_base 14: mod_base = OTHER; note = thymine
  modified_base 17: mod_base = OTHER; note = thymine
  misc_feature 1..20: note = Rabbit VEGFA sgRNA5
  source 1..20: mol_type = other RNA
tagggggcgagggtgctgcg

SEQ ID NO: 65 - RNA, 20 nt, synthetic construct
  modified_base 2: mod_base = OTHER; note = thymine
  modified_base 9..10: mod_base = OTHER; note = thymine
  modified_base 19: mod_base = OTHER; note = thymine
  misc_feature 1..20: note = Rabbit VEGFA sgRNA6
  source 1..20: mol_type = other RNA
ctccagggttccaacagctc

SEQ ID NO: 66 - RNA, 20 nt, synthetic construct
  modified_base 1: mod_base = OTHER; note = thymine
  modified_base 3: mod_base = OTHER; note = thymine
  modified_base 5: mod_base = OTHER; note = thymine
  modified_base 13: mod_base = OTHER; note = thymine
  modified_base 15: mod_base = OTHER; note = thymine
  modified_base 17: mod_base = OTHER; note = thymine
  misc_feature 1..20: note = Rabbit VEGFA sgRNA7
  source 1..20: mol_type = other RNA
tgtgtgagcgcgtgtgtagg

SEQ ID NO: 67 - RNA, 20 nt, synthetic construct
  modified_base 13: mod_base = OTHER; note = thymine
  modified_base 16: mod_base = OTHER; note = thymine
  modified_base 20: mod_base = OTHER; note = thymine
  misc_feature 1..20: note = Rabbit VEGFA sgRNA8
  source 1..20: mol_type = other RNA
agggggcgagggtgctgcgt

SEQ ID NO: 68 - RNA, 20 nt, synthetic construct
  modified_base 18: mod_base = OTHER; note = thymine
  modified_base 20: mod_base = OTHER; note = thymine
  misc_feature 1..20: note = Rabbit VEGFA sgRNA9
  source 1..20: mol_type = other RNA
gggaaggggcccagagctgt

SEQ ID NO: 69 - RNA, 20 nt, synthetic construct
  modified_base 13: mod_base = OTHER; note = thymine
  misc_feature 1..20: note = Rabbit VEGFA sgRNA10
  source 1..20: mol_type = other RNA
gcggggagcccgtcagggac

SEQ ID NO: 70 - RNA, 20 nt, synthetic construct
  modified_base 2: mod_base = OTHER; note = thymine
  modified_base 6: mod_base = OTHER; note = thymine
  modified_base 14: mod_base = OTHER; note = thymine
  misc_feature 1..20: note = Rabbit VEGFA sgRNA11
  source 1..20: mol_type = other RNA
gtccctgacgggctccccgc

SEQ ID NO: 71 - RNA, 20 nt, synthetic construct
  modified_base 14: mod_base = OTHER; note = thymine
  modified_base 18: mod_base = OTHER; note = thymine
  misc_feature 1..20: note = Rabbit VEGFA sgRNA12
  source 1..20: mol_type = other RNA
aggaggccgggggtcactcc

SEQ ID NO: 72 - RNA, 21 nt, synthetic construct
  modified_base 3: mod_base = OTHER; note = thymine
  modified_base 7: mod_base = OTHER; note = thymine
  misc_feature 1..21: note = Rabbit VEGFA sgRNA13
  source 1..21: mol_type = other RNA
gctgcgtggggaggaggccgg

SEQ ID NO: 73 - RNA, 20 nt, synthetic construct
  modified_base 1: mod_base = OTHER; note = thymine
  modified_base 8..9: mod_base = OTHER; note = thymine
  modified_base 18: mod_base = OTHER; note = thymine
  modified_base 20: mod_base = OTHER; note = thymine
  misc_feature 1..20: note = Rabbit VEGFA sgRNA14
  source 1..20: mol_type = other RNA
tccagggttccaacagctct

SEQ ID NO: 74 - RNA, 20 nt, synthetic construct
  modified_base 18: mod_base = OTHER; note = thymine
  misc_feature 1..20: note = Rabbit VEGFA sgRNA15
  source 1..20: mol_type = other RNA
gacgggcggggagcccgtca

SEQ ID NO: 75 - RNA, 20 nt, synthetic construct
  modified_base 2: mod_base = OTHER; note = thymine
  modified_base 18..19: mod_base = OTHER; note = thymine
  misc_feature 1..20: note = Rabbit VEGFA sgRNA16
  source 1..20: mol_type = other RNA
gtgaggagcgcagaggcttg

SEQ ID NO: 76 - RNA, 20 nt, synthetic construct
  modified_base 9: mod_base = OTHER; note = thymine
  misc_feature 1..20: note = Rabbit VEGFA sgRNA17
  source 1..20: mol_type = other RNA
gggagcggtgaggagcgcag

SEQ ID NO: 77 - RNA, 20 nt, synthetic construct
  modified_base 1: mod_base = OTHER; note = thymine
  modified_base 5: mod_base = OTHER; note = thymine
  modified_base 10: mod_base = OTHER; note = thymine
  modified_base 19: mod_base = OTHER; note = thymine
  misc_feature 1..20: note = Rabbit VEGFA sgRNA18
  source 1..20: mol_type = other RNA
tcagtcgcctgggagcggtg

SEQ ID NO: 78 - RNA, 20 nt, synthetic construct
  modified_base 14: mod_base = OTHER; note = thymine
  misc_feature 1..20: note = Rabbit VEGFA sgRNA19
  source 1..20: mol_type = other RNA
ggggcagccgggctgcgagg

SEQ ID NO: 79 - RNA, 20 nt, synthetic construct
  modified_base 3: mod_base = OTHER; note = thymine
  modified_base 19..20: mod_base = OTHER; note = thymine
  misc_feature 1..20: note = Rabbit VEGFA sgRNA20
  source 1..20: mol_type = other RNA
ggtgaggagcgcagaggctt

SEQ ID NO: 80 - RNA, 20 nt, synthetic construct
  misc_feature 1..20: note = Rabbit VEGFA sgRNA21
  source 1..20: mol_type = other RNA
cgcagggggagcggagccgg

SEQ ID NO: 81 - RNA, 20 nt, synthetic construct
  misc_feature 1..20: note = Rabbit VEGFA sgRNA22
  source 1..20: mol_type = other RNA
gcgaggaggccgcggcgcag

SEQ ID NO: 82 - RNA, 20 nt, synthetic construct
  misc_feature 1..20: note = Rabbit VEGFA sgRNA23
  source 1..20: mol_type = other RNA
cgaggaggccgcggcgcagg

SEQ ID NO: 83 - RNA, 20 nt, synthetic construct
  modified_base 3: mod_base = OTHER; note = thymine
  modified_base 8: mod_base = OTHER; note = thymine
  modified_base 14: mod_base = OTHER; note = thymine
  misc_feature 1..20: note = Rabbit VEGFA sgRNA24
  source 1..20: mol_type = other RNA
gctccgctccccctgcgccg

SEQ ID NO: 84 - RNA, 20 nt, synthetic construct
  misc_feature 1..20: note = Rabbit VEGFA sgRNA25
  source 1..20: mol_type = other RNA
  modified_base 2: mod_base = OTHER; note = thymine
  modified_base 7: mod_base = OTHER; note = thymine
  modified_base 10: mod_base = OTHER; note = thymine
  modified_base 17: mod_base = OTHER; note = thymine
ctgcgctcctcaccgctccc

(19) United States
(12) Patent Application Publication
(10) Pub. No.: US 2025/0066772 A1
(43) Pub. Date: Feb. 27, 2025

(54) METHOD OF MODULATING VEGF AND USES THEREOF

(71) Applicant: Epigenic Therapeutics, Inc., Shanghai (CN)

(72) Inventors: Changyang ZHOU, Shanghai (CN); Yidi SUN, Shanghai (CN); Shaoshuai MAO, Shanghai (CN); Wenbo PENG, Shanghai (CN)

(21) Appl. No.: 18/724,421

(22) PCT Filed: Jan. 10, 2023
(86) PCT No.: PCT/CN2023/071521

§ 371 Date: Jun. 26, 2024

(30) Foreign Application Priority Data
Jan. 14, 2022 (WO) PCT/CN2022/071930

(51) Int. Cl. C12N 15/11; C12N 9/10; C12N 9/22; C12N 15/86; C12N 15/88; C12N 15/90

(52) U.S. Cl. CPC C12N 15/11; C12N 9/1007; C12N 9/22; C12N 15/86; C12N 15/88; C12N 15/907; C12Y 201/01037; C07K 2319/80; C12N 2310/20; C12N 2750/14143

(57) ABSTRACT

The invention provides for a composition, which in particular comprising a fusion molecule comprising a least one DNA binding protein and at least one modulator of gene expression, wherein the fusion molecule is targeted to a genomic region near a VEGF gene and/or within a VEGF regulatory element and the at least one modulator of gene expression provides a modification of at least one nucleotide near the VEGF gene and/or within a VEGF regulatory element. The invention also provides methods of reduction or elimination of VEGF gene products in vivo and uses thereof.

### FIELD

The present disclosure relates generally to the fields of molecular biology, immunology, and medicine. More particularly, it relates to CRISPR/Cas9 based fusion molecules for use in targeted reduction or elimination of VEGF gene products in vivo and methods of use thereof.

### SEQUENCE LISTING

The application comprises a Sequence Listing, filed herewith in electronic format, and is hereby incorporated into the specification by reference.

### BACKGROUND

Engineered DNA-binding proteins that can be customized to target any gene in mammalian cells have enabled rapid advances in biomedical research and are a promising platform for gene therapies. The RNA-guided CRISPR-Cas9 system has emerged as a promising platform for programmable targeted gene regulation. Fusion of catalytically inactive, “dead” Cas9 (dCas9) to the Kruppel-associated box (KRAB) domain generates a synthetic repressor capable of highly specific and potent modulation or silencing of target genes in cell culture experiments.

However, persistent modulation and silencing of endogenous genes using synthetic dCas9-KRAB fusion proteins have presented challenges for use in in vivo therapies. Synthetic repressors exceed size packaging limits of viral vector delivery methods. Safety, toxicity, immunogenicity, and off-target effects are other challenges that limit the use of synthetic repressors in vivo.

There is a need in the art for alternative approaches for generating genetically engineered synthetic gene repressors and in vivo delivery of the synthetic gene repressors for use as therapeutics. The present disclosure addresses this unmet need in the art.

### SUMMARY

In an aspect, the disclosure provides a sgRNA, comprising a sequence complementary to a target DNA sequence located within 500 bp upstream to 500 bp downstream of the transcription start site of Vascular Endothelial Growth Factor (VEGF) gene.

In some embodiments, the sgRNA comprises the nucleic acid sequence of any one of SEQ ID NOs: 29-58 and 60-84.

In some embodiments, the VEGF gene is VEGF-A gene from a mammalian animal, such as human, monkey, mouse, rat, and rabbit.

In an aspect, the disclosure provides a DNA sequence encoding the sgRNA as disclosed herein.

In an aspect, the disclosure provides a composition comprising:
(a) a fusion molecule comprising a least one DNA binding protein and at least one modulator of gene expression, or a nucleic acid sequence encoding the fusion molecule; and
(b) a guiding molecule comprising the sgRNA as disclosed herein and a protein binding sequence that is capable of binding to the at least one DNA binding protein, or a nucleic acid sequence encoding the guiding molecule;
wherein the at least one modulator of gene expression provides a modification of at least one nucleotide near the VEGF gene and/or within a VEGF regulatory element.

In some embodiments, the at least one modulator of gene expression as described herein provides a modification of at least one nucleotide from within 1,000 bp upstream to 1,000 bp downstream of the transcription start site of the VEGF gene.

In an aspect, the disclosure provides a composition comprising a fusion molecule comprising a least one DNA binding protein and at least one modulator of gene expression, or a nucleic acid sequence encoding the fusion molecule, wherein the fusion molecule is targeted to a genomic region near a VEGF gene and/or within a VEGF regulatory element, wherein the at least one modulator of gene expression provides a modification of at least one nucleotide near the VEGF gene and/or within a VEGF regulatory element.

In some embodiments, the at least one modulator of gene expression comprises a DNA methyltransferase (DNMT), a DNA demethylase, a histone methyltransferase, a histone demethylase, or a portion thereof, or a zinc finger protein-based transcription factor or a portion thereof, or a combination thereof.

In some embodiments, the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

In some embodiments, the VEGF gene is VEGF-A, VEGF-B, VEGF-C, VEGF-D or VEGF-E gene.

In some embodiments, the VEGF regulatory element is a transcription start site, core promoter, a proximal promoter, a distal enhancer, a silencer, an insulator element, a boundary element or a locus control region.

In some embodiments, the modification of at least one nucleotide near the VEGF gene and/or within a VEGF regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream or downstream of the transcription start site of the VEGF gene.

In some embodiments, the modification of at least one nucleotide near the VEGF gene and/or within a VEGF regulatory element is located within 500 bp upstream or downstream of the transcription start site of the VEGF gene.

In some embodiments, the modification of at least one nucleotide near the VEGF gene and/or within a VEGF regulatory element is located within 300 bp upstream or downstream of the transcription start site of the VEGF gene.

In some embodiments, the modification of at least one nucleotide near the VEGF gene and/or within a VEGF regulatory element is located within 1000 bp upstream of the transcription start site to within 300 bp downstream of the transcription start site of the VEGF gene.

In some embodiments, the modification of at least one nucleotide is a DNA methylation.

In some embodiments, the at least one modulator of gene expression comprises one or more selected from a DNA methyltransferase (DNMT), a zinc-finger protein-based transcription factor, a portion thereof and any combinations thereof. The DNA methyltransferase may be DNMT3A, DNMT3B, DNMT3L, DNMT1 and/or DNMT2.

In some embodiments, the DNMT3A comprises the amino acid sequence of SEQ ID NO: 23, and/or the DNMT3L comprises the amino acid sequence of SEQ ID NO: 24.

In some embodiments, the zinc finger protein-based transcription factor is Kruppel-associated suppression box (KRAB). Specifically, the KRAB may comprise the amino acid sequence of SEQ ID NO: 22.

In some embodiments, the at least one modulator of gene expression comprises a DNA methyltransferase or a portion thereof, and a zinc finger protein-based transcription factor or a portion thereof. Specifically, the DNA methyltransferase may be selected from DNMT3A and DNMT3L and a combination thereof, and the zinc finger protein-based transcription factor may be KRAB.

In some embodiments, the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease. For example, the at least one DNA binding protein is dCas9.

In some embodiments, the dCas9 comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, a Streptococcus thermophilus (e.g., strain LMD-9) dCas9.

In some embodiments, the dCas9 comprises the amino acid sequence of SEQ ID NO: 1.

In some embodiments, the fusion molecule as disclosed herein comprises the at least one modulator of gene expression fused to the C-terminus, the N-terminus, or both, of the at least one DNA binding protein.

In some embodiments, the at least one modulator of gene expression is fused directly to the at least one DNA binding protein.

In some embodiments, the at least one modulator of gene expression is fused indirectly with the at least one DNA binding protein via a non-modulator, a second modulator, or a linker.

In some embodiments, the fusion molecule as disclosed herein comprises a dCas9 fused with a KRAB on the C-terminal end and a DNMT3A and a DNMT3L on the N-terminal end.

In some embodiments, the fusion molecule comprises the amino acid sequence of SEQ ID NO: 28.

In some embodiments, the fusion molecule further comprises at least one nuclear localization sequence. The at least one nuclear localization sequence may be directly or indirectly fused to the C-terminus, the N-terminus or both of the at least one DNA binding protein.

In some embodiments, the nucleic acid sequence encoding the fusion molecule is a deoxyribonucleic acid (DNA) or a messenger ribonucleic acid (mRNA).

In some embodiments, the composition as disclosed herein further comprises at least one single guide RNA (sgRNA) that is complementary to a target DNA sequence near the VEGF gene and/or within a VEGF regulatory element.

In some embodiments, the target DNA sequence is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream or downstream of the transcription start site of the VEGF gene.

In some embodiments, the sgRNA comprises the nucleic acid sequence of SEQ ID NOs: 29-58 and 60-84.

In some embodiments, the fusion molecule is packaged in a liposome or a lipid nanoparticle.

In some embodiments, the fusion molecule and the sgRNA are packaged in a liposome or a lipid nanoparticle. The fusion molecule and the sgRNA may be packaged in the same liposome or lipid nanoparticle, or in different liposomes or lipid nanoparticles.

In some embodiments, the liposome or the lipid nanoparticle comprises of ionizable lipids (20%-70%, molar ratio), PEGylated lipids (0%-30%, molar ratio), supporting lipids (30%-50%, molar ratio), and cholesterol (10%-50%, molar ratio).

In some embodiments, the ionizable lipid is selected from a group consisting of pH-responsive ionizable lipids, thermal-responsive ionizable lipids and light-responsive ionizable lipids.

In some embodiments, the fusion molecule is packaged in an AAV vector.

In some embodiments, the fusion molecule and the sgRNA are packaged in an AAV vector. The fusion molecule and the sgRNA may be packaged in the same AAV vector or in different AAV vectors.

In some embodiments, the composition as disclosed herein is a pharmaceutical composition comprising a pharmaceutically acceptable carrier.

In an aspect, the disclosure provides a method for modulating (e.g., reducing or eliminating) the expression of a VEGF gene product in a cell comprising the step of introducing into the cell: a fusion molecule comprising a least one DNA binding protein and at least one modulator of gene expression, or a nucleic acid sequence encoding the fusion molecule, wherein the modulator of gene expression provides a modification of at least one nucleotide near the VEGF gene and/or within a VEGF regulatory element, thereby modulating (e.g., reducing or eliminating) the expression of the VEGF gene product in the cell.

In an aspect, the disclosure provides an in vivo method of modulating (e.g., reducing or eliminating) the expression of a VEGF gene product in a subject, comprising the step of introducing to a cell of the subject: a fusion molecule comprising a least one DNA binding protein and at least one modulator of gene expression, or a nucleic acid sequence encoding the fusion molecule, wherein the modulator of gene expression provides a modification of at least one nucleotide near the VEGF gene and/or within a VEGF regulatory element, thereby modulating (e.g., reducing or eliminating) the expression of the VEGF gene product in the subject.

In an aspect, the disclosure provides a method for treating or alleviating a symptom of a VEGF related disorder in a subject, comprising the step of introducing to a cell of the subject: a fusion molecule comprising a least one DNA binding protein and at least one modulator of gene expression, or a nucleic acid sequence encoding the fusion molecule, wherein the modulator of gene expression provides a modification of at least one nucleotide near the VEGF gene and/or within a VEGF regulatory element, thereby treating or alleviating a symptom of a VEGF related disorder in the subject.

In some embodiments, the VEGF regulatory element is a core promoter, a proximal promoter, a distal enhancer, a silencer, an insulator element, a boundary element or a locus control region.

In some embodiments, the modification of at least one nucleotide near the VEGF gene and/or within a VEGF regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the VEGF gene. In some embodiments, the modification of at least one nucleotide near the VEGF gene and/or within a VEGF regulatory element is located within 1000 bp upstream of the transcription start site of the VEGF gene. In some embodiments, the modification of at least one nucleotide near the VEGF gene and/or within a VEGF regulatory element is located within 300 bp upstream of the transcription start site of the VEGF gene.

In some embodiments, the modification of at least one nucleotide near the VEGF gene and/or within a VEGF regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the VEGF gene. In some embodiments, the modification of at least one nucleotide near the VEGF gene and/or within a VEGF regulatory element is located within about 300 bp downstream of the transcription start site of the VEGF gene. In some embodiments, the modification of at least one nucleotide near the VEGF gene and/or within a VEGF regulatory element is located within 1000 bp upstream of the transcription start site and within 300 bp downstream of the transcription start site of the VEGF gene.

In some embodiments, the modification of at least one nucleotide is a DNA methylation.

In some embodiments, the at least one modulator of gene expression comprises a DNA methyltransferase (DNMT), a DNA demethylase, a histone methyltransferase, a histone demethylase, or a portion thereof.

In some embodiments, the at least one modulator of gene expression comprises a DNA methyltransferase (DNMT) or a portion thereof. In some embodiments, the DNA methyltransferase is DNMT3A, DNMT3B, DNMT3L, DNMT1 or DNMT2. In some embodiments, the DNMT3A comprises the amino acid sequence of SEQ ID NO: 23. In some embodiments, the DNMT3L comprises the amino acid sequence of SEQ ID NO: 24.

In some embodiments, the at least one modulator of gene expression comprises a zinc finger protein-based transcription factor or a portion thereof. In some embodiments, the zinc finger protein-based transcription factor is Kruppel-associated suppression box (KRAB). In some embodiments, the KRAB comprises the amino acid sequence of SEQ ID NO: 22.

In some embodiments, the at least one modulator of gene expression comprises a DNA methyltransferase or a portion thereof and a zinc finger protein-based transcription factor or a portion thereof. In some embodiments, the DNA methyltransferase is selected from DNMT3A and DNMT3L and a combination thereof, and the zinc finger protein-based transcription factor is KRAB.

In some embodiments, the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease. In some embodiments, the at least one DNA binding protein is dCas9. In some embodiments, the dCas9 comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, a Streptococcus thermophilus (e.g., strain LMD-9) dCas9. In some embodiments, the dCas9 comprises the amino acid sequence of SEQ ID NO: 1.

In some embodiments, the fusion molecule comprises the at least one modulator of gene expression fused to the C-terminus, the N-terminus, or both, of the at least one DNA binding protein.

In some embodiments, the at least one modulator of gene expression is fused directly to the at least one DNA binding protein. In some embodiments, the at least one modulator of gene expression is fused indirectly with the at least one DNA binding protein via a non-modulator, a second modulator, or a linker. In some embodiments, the fusion molecule comprises a dCas9 fused with a KRAB on the C-terminal end and a DNMT3A and a DNMT3L on the N-terminal end. In some embodiments, the fusion molecule comprises the amino acid sequence of SEQ ID NO: 28.

In some embodiments, the fusion molecule further comprises at least one nuclear localization sequence. In some embodiments, the at least one nuclear localization sequence is directly fused to the C-terminus, the N-terminus or both of the at least one DNA binding protein. In some embodiments, the at least one nuclear localization sequence is indirectly fused to the C-terminus, the N-terminus or both of the at least one DNA binding protein via a linker.

In some embodiments, the nucleic acid sequence encoding the fusion molecule is a deoxyribonucleic acid (DNA). In some embodiments, the nucleic acid sequence encoding the fusion molecule is a messenger ribonucleic acid (mRNA).

In some embodiments, the method further comprises the step of introducing at least one single guide RNA (sgRNA) that is complementary to a DNA sequence near the VEGF gene and/or within a VEGF regulatory element, thereby targeting the fusion molecule to the VEGF gene or VEGF regulatory element, or a DNA encoding the sgRNA. In some embodiments, the sgRNA comprises the nucleic acid sequence of SEQ ID Nos: 29-58 and 60-84.

In some embodiments, the fusion molecule is formulated in a liposome or a lipid nanoparticle. In some embodiments, the fusion molecule and the sgRNA are formulated in a liposome or a lipid nanoparticle. In some embodiments, the fusion molecule and the sgRNA are formulated in the same liposome or lipid nanoparticle. In some embodiments, the fusion molecule and the sgRNA are formulated in different liposome or lipid nanoparticle.

In some embodiments, the liposome or lipid nanoparticle comprises of ionizable lipids (20%-70%, molar ratio), PEGylated lipids (0%-30%, molar ratio), supporting lipids (30%-50%, molar ratio), and cholesterol (10%-50%, molar ratio). In some embodiments, the ionizable lipid is selected from a group consisting of pH-responsive ionizable lipids, thermal-responsive ionizable lipids and light-responsive ionizable lipids.

In some embodiments, the fusion molecule is formulated in an AAV vector. In some embodiments, the fusion molecule and the sgRNA are formulated in an AAV vector. In some embodiments, the fusion molecule and the sgRNA are formulated in the same AAV vector. In some embodiments, the fusion molecule and the sgRNA are formulated in different AAV vectors.

In some embodiments, the fusion molecule is delivered to the cell by local injection, systemic infusion, or a combination thereof. In some embodiments, the fusion molecule is delivered to the eye of the subject by intraocular injection or intravitreal injection.

In some embodiments, the subject is a mammalian, such as human, monkey, mouse, rat, rabbit, pig, horse, cat and dog.

In some embodiments, the VEGF related disorder is associated with angiogenesis. In some embodiments, the VEGF related disorder is neovascular disorder, such as an ocular neovascular disorder, including age related macular degeneration (AMD). In some embodiments, the VEGF related disorder is wet AMD or dry AMD.

The disclosure provides a sgRNA comprising the nucleic acid sequence of any one of SEQ ID NOs: 29-58 and 60-84. The disclosure provides a DNA sequence encoding any one of the sgRNA disclosed herein.

In an aspect, provided herein is the composition as described above for use in treating or alleviating a symptom of a VEGF related disorder in a subject.

In some embodiments, the VEGF related disorder is neovascular disorder, such as an ocular neovascular disorder, including age related macular degeneration (AMD).

In an aspect, provided herein is use of the composition as described above in the manufacture of a medicament for treating or alleviating a symptom of a VEGF related disorder in a subject.

In an aspect, provided herein is a kit, comprising a container that comprises the composition or the fusion molecule as described above.

### BRIEF DESCRIPTION OF THE DRAWINGS

FIG. 1A is a schematic diagram showing the “EPICAS” dual plasmid system and a sgRNA tiling screen design targeting mouse VEGF-A expression. The first plasmid (“catalytic protein” plasmid or “fusion molecule” plasmid), encodes DNMT3A-DNMT3L (3A3L)-dCas9-KRAB, under the control of a CAG promoter, and a GFP marker separated by 2A elements. The second plasmid (“sgRNA” plasmid) has an sgRNA-scaffold under the control of a U6 promoter and a mCherry marker under the control of a CMV promoter. The sgRNAs of the tiling screen target the transcription start site (TSS)+250 bp upstream of the mouse VEGF-A protein coding sequence (CDS).

FIG. 1B is a bar graph showing relative mRNA expression 48 hours following transfection of a mouse N2A cell line with the catalytic protein plasmid and various single VEGF sgRNA plasmids.

FIG. 1C is a bar graph showing the relative mRNA expression one week following transfection of a mouse N2A cell line with the catalytic protein plasmid and various single VEGF sgRNA plasmids or a mixture of VEGF sgRNA plasmids.

FIG. 1D is a schematic diagram showing the bisulfite PCR analysis result of the VEGF-A locus. Each row represents one single clone, and each column indicates one specific genomic position. Black dots represent sites with successful methylation.

FIG. 2A is a schematic diagram showing the EPICAS mRNA plasmid design. The EPICAS ORF comprises a DNMT3A-DNMT3L-dCas9-KRAB cassette. The plasmid can be digested at the XbaI and BpiI restriction sites to form a linearized plasmid.

FIG. 2B is an electrophoretogram of purified mRNA expressed from the EPICAS mRNA plasmid.

FIG. 2C is a schematic diagram showing EPICAS mRNA can successfully knock down the endogenous VEGFA gene in primary mouse hepatocytes. Left panel, a microscopic photograph of primary mouse hepatocytes; middle panel, flow cytometry graphs showing GFP expression 72 hours post-transfection without or with GFP-P2A-Casoff mRNA and sgRNA treatment; right panel, relative VEGFA mRNA expression in GFP positive cells between control and GFP-P2A-Casoff mRNA and sgRNA treated groups.

FIG. 3A is a schematic diagram of a lipid nanoparticle (LNP) design. Epigenetic CRISPR/Cas elements and sgRNA elements may be encapsulated by LNPs.

FIG. 3B is a transmission electron microscope image showing LNPs containing EPICAS.

FIG. 3C is a graph showing the size distribution of LNPs.

FIG. 3D are a series of pictures showing in vivo fluorescence imaging of luciferase mRNAs delivered by lipid nanoparticle to the eyes of Ai9 mice by intravitreal injection.

FIG. 3E is a schematic diagram of an in vivo experimental design for delivery of LNPs containing EPICAS mRNA and mouse VEGF targeting sgRNA. LNPs were administered to the Ai9 mice via injection into eye posterior region and VEGFA gene expression in the retina and choroid membrane was analyzed 5 days post injection.

FIG. 4A is a schematic diagram of an sgRNA tiling screen design for rabbit VEGFA in 293T reporter cell line. The sgRNAs of the tiling screen target 500 bp upstream and downstream of the transcription start site (TSS) of rabbit VEGFA gene.

FIG. 4B is a series of graphs showing the fluorescence intensity of the reporter cells 72 hours following transfection with EPICAS plasmids and sgRNA targeting rabbit VEGFA.

FIG. 4C is a series of graphs showing the mRNA expression of VEGFA in rabbit RK-13 cells following the transfection of six sgRNA that had good knockdown effects together with EPICAS plasmids, which targeted the endogenous gene VEGFA in the rabbit cells.

FIG. 5A is schematic diagram showing the experimental design of an sgRNA tiling screen targeting conserved regions in human and mouse VEGF-A, specifically, within 300 bp upstream to 300 bp downstream of the transcription start site (TSS) of human VEGFA gene.

FIG. 5B is a series of graphs showing the VEGFA mRNA expression 48 hours following transfection with EPICAS dual plasmid system using various sgRNAs targeting VEGF.

FIG. 5C is a series of graphs showing the VEGFA mRNA expression 96 hours following transfection with EPICAS dual plasmid system using various sgRNAs targeting VEGF.

### DETAILED DESCRIPTION

The present disclosure overcomes problems associated with current technologies by providing genetically engineered fusion molecules (e.g., DNMT3A-DNMT3L (3A3L)-dCas9-KRAB fusion molecule) for targeted reduction or elimination of gene products (e.g., VEGF) in a cell for use in in vivo gene therapy. The genetically engineered fusion molecules of the disclosure are useful for treatment of genetic diseases, including for example, diseases of the liver, diseases associated with high cholesterol, and diseases associated with dysregulation of cholesterol (e.g. low density lipoprotein (LDL) cholesterol). Accordingly, methods of making genetically engineered fusion molecules and pharmaceutical formulations thereof (e.g., lipid nanoparticle formulations) for use in in vivo delivery are also provided.

### I. Definitions

As used herein, the term “coding sequence” or “encoding nucleic acid” means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

The term “complement” or “complementary” as used herein with reference to a nucleic acid can mean Watson-Crick (e.g., A-T/U and C-G) or Hoogsteen base pairing between nucleotides or nucleotide analogs of nucleic acid molecules. “Complementarity” refers toa property shared between two nucleic acid sequences, such that when they are aligned antiparallel to each other, the nucleotide bases at each position will be complementary.

The term “correcting”, “genome editing” and “restoring” refers to changing a mutant gene that encodes a mutant protein, a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

As used herein, the term “donor DNA”, “donor template” and “repair template” refers to a double-stranded DNA fragment or molecule that includes at least a portion of the gene of interest. The donor DNA may encode a full-functional protein or a partially-functional protein.

As used herein, the terms “frameshift” or “frameshift mutation” are used interchangeably and refer to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

As used herein, the term “functional” and “full-functional” describes a protein that has biological activity. A “functional gene” refers to a gene transcribed to mRNA, which is translated to a functional protein.

As used herein, the term “fusion protein” refers to a chimeric protein created through the covalent or non-covalent joining of two or more genes, directly or indirectly, that originally coded for separate proteins. In some embodiments, the translation of the fusion gene results in a single polypeptide with functional properties derived from each of the original proteins.

As used herein, the term “genetic construct” refers to the DNA or RNA molecules that comprise a nucleotide sequence that encodes a protein. The coding sequence includes initiation and termination signals operably linked to regulatory elements including a promoter and polyadenylation signal capable of directing expression in cells.

The term “Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, nonhomologous end joining may take place instead.

The term “genome editing” as used herein refers to changing a gene. Genome editing may include correcting or restoring a mutant gene. Genome editing may include knocking out a gene, such as a mutant gene or a normal gene. Genome editing may be used to treat disease by changing the gene of interest.

The term “identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al, SIAM J. Applied Math. 48, 1073 (1988), herein incorporated by reference in their entirety.

As used herein, the terms “mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

As used herein, the term “modulator of epigenetic modification” refers to an agent that targets gene expression via epigenetic modification (e.g., via histone acetylation or methylation, or DNA methylation at a regulatory element of target gene, e.g., a promoter, enhancer or transcription start site). Chromatin remodeling and DNA methylation are two main mechanisms for regulating gene transcription. Specific epigenetic marks (e.g., DNA methylation) structurally or biochemically direct gene transcription or gene silencing/repression. For example, DNA methylation of regions that regulate transcriptional activities alter gene expression without changing the underlying DNA sequence. Transcriptional regulation using epigenetic modification (e.g., DNA methylation) allows for targeted modulation of gene expression, without affecting the expression of other gene products.

The term “non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

The term “normal gene” as used herein refers to a gene that has not undergone a change, such as a loss, gain, or exchange of genetic material. The normal gene undergoes normal gene transmission and gene expression.

The term “nuclease mediated NHEJ” as used herein refers to NHEJ that is initiated after anuclease, such as a cas9, cuts double stranded DNA.

As used herein, the term “nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, iso-cytosine and iso-guanine. Nucleic acids may be obtained by chemical synthesis methods or by recombinant methods.

As used herein, the term “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. When used with respect to polynucleotides, for one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide. In some embodiments, the expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “partially-functional” as used herein describes a protein that is encoded by a mutant gene and has less biological activity than a functional protein but more than a non-functional protein. In one embodiment, a partially-functional protein shows a biological activity that is less than 95%, 90%, 85%, 80%, 75%, 70%, 65%, 60%, 55%, 50%, 45%, 40%, 35%, or 30% of that of a corresponding functional protein.

The term “premature stop codon” or “out-of-frame stop codon” as used interchangeably herein refers to nonsense mutation in a sequence of DNA, which results in a stop codon at a location not normally found in the wild-type gene. A premature stop codon may cause a protein to be truncated or shorter compared to the full-length version of the protein.

The term “promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of a nucleic acid. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, and CMV IE promoter.

The term “target gene” as used herein refers to any nucleotide sequence encoding a known or putative gene product. The target gene may be a mutated gene involved in a genetic disease or disorder.

The term “target region” as used herein refers to the region of the target gene to which the site-specific nuclease is designed to bind.

As used herein, the term “transgene” refers to a gene or genetic material containing a gene sequence that has been isolated from one organism and is introduced into a different organism. Alternatively, the term “transgene” also refers to a gene or genetic material that is chemically synthesized and introduced into an organism. This non-native segment of DNA may retain the ability to produce RNA or protein in the transgenic organism, or it may alter the normal function of the transgenic organism's genetic code. The introduction of a transgene has the potential to change the phenotype of an organism.

As used herein, the term “variant” when used with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity.

Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982), incorporated herein by reference in its entirety. The hydropathic index of an amino acid is based on a consideration of its hydrophobicity and charge. It is known in the art that amino acids of similar hydropathic indexes may be substituted and still retain protein function. In one aspect, amino acids having hydropathic indexes of +2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, the term “vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, such as a DNA plasmid.

As used herein, the terms “gene transfer,” “gene delivery,” and “gene transduction” refer to methods or systems for reliably inserting a particular nucleotide sequence (e.g., DNA or RNA), fusion protein, polypeptide and the like into targeted cells.

As used herein, the terms “adenoviral associated virus (AAV) vector,” “AAV gene therapy vector,” and “gene therapy vector” refer to a vector having functional or partly functional ITR sequences and transgenes. As used herein, the term “ITR” refers to inverted terminal repeats (ITR). The ITR sequences may be derived from an adeno-associated virus serotype, including without limitation, AAV-1, AAV-2, AAV-3, AAV-4, AAV-5, and AAV-6. The ITRs, however, need not be the wild-type nucleotide sequences, and may be altered (e.g., by the insertion, deletion or substitution of nucleotides), so long as the sequences retain function to provide for functional rescue, replication and packaging. AAV vectors may have one or more of the AAV wild-type genes deleted in whole or part, preferably the rep and/or cap genes but retain functional flanking ITR sequences. Functional ITR sequences function to, for example, rescue, replicate and package the AAV virion or particle. Thus, an “AAV vector” is defined herein to include at least those sequences required for insertion of the transgene into a subject's cells. Optionally included are those sequences necessary in cis for replication and packaging (e.g., functional ITRs) of the virus.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

The “transgene” may contain a transgenic sequence or a native or wild-type DNA sequence. The transgene may become part of the genome of the primate subject. A transgenic sequence can be partly or entirely species-heterologous, i.e., the transgenic sequence, or a portion thereof, can be from a species which is different from the cell into which it is introduced.

As used herein, the term “stably maintained” refers to characteristics of transgenic subject (e.g., a human or non-human primate) that maintain at least one of their transgenic elements (i.e., the element that is desired) through multiple generations of cells. For example, it is intended that the term encompass many cell division cycles of the originally transfected cell. The term “stable transfection” or “stably transfected” refers to the introduction and integration of foreign DNA into the genome of the cell. The term “stable transfectant” refers to a cell that has stably integrated foreign DNA into the genomic DNA.

As used herein, the terms “transgene encoding,” “nucleic acid molecule encoding,” “DNA sequence encoding,” and “DNA encoding” refer to the order or sequence of deoxyribonucleotides along a strand of deoxyribonucleic acid. The order of these deoxyribonucleotides may, for example, determine the order of amino acids along the polypeptide (protein) chain. The DNA sequence thus may code for the amino acid sequence.

As used herein, the term “wild type” (wt) refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants may be isolated, which are identified by the acquisition of altered characteristics when compared to the wild-type gene or gene product.

As used herein, the term “transfection” refers to the uptake of a foreign nucleic acid (e.g., DNA or RNA) by a cell. A cell has been “transfected” when an exogenous nucleic acid (DNA or RNA) has been introduced inside the cell membrane. A number of transfection techniques are generally known in the art (See, e.g., Graham et al., Virol., 52:456 (1973); Sambrook et al., Molecular Cloning, a Laboratory Manual, Cold Spring Harbor Laboratories, New York (1989); Davis et al., Basic Methods in Molecular Biology, Elsevier, (1986); and Chu et al., Gene 13:197 (1981), incorporated herein by reference in their entirety). Such techniques may be used to introduce one or more exogenous DNA moieties, such as a gene transfer vector and other nucleic acid molecules, into suitable recipient cells.

As used herein, the terms “stable transfection” and “stably transfected” refers to the introduction and integration of foreign DNA into the genome of the transfected cell. The term “stable transfectant” refers to a cell, which has stably integrated foreign DNA into the genomic DNA.

As used herein, the term “transient transfection” or “transiently transfected” refers to the introduction of foreign DNA into a cell wherein the foreign DNA fails to integrate into the genome of the transfected cell and is maintained as an episome. During this time the foreign DNA is subject to the regulatory controls that govern the expression of endogenous genes in the chromosomes. The term “transient transfectant” refers to cells which have taken up foreign DNA but have failed to integrate this DNA. As used herein, the term “transduction” denotes the delivery of a DNA molecule to a recipient cell either in vivo or in vitro, via a replication-defective viral vector, such as via a recombinant AAV virion.

As used herein, the term “recipient cell” refers to a cell which has been transfected or transduced, or is capable of being transfected or transduced, by a nucleic acid construct or vector bearing a selected nucleotide sequence of interest. The term includes the progeny of the parent cell, whether or not the progeny are identical in morphology or in genetic make-up to the original parent, so long as the selected nucleotide sequence is present. The recipient cell may be the cells of a subject to which the gene therapy particles and/or gene therapy vector has been administered.

As used herein, the term “recombinant DNA molecule” refers to a DNA molecule which is comprised of segments of DNA joined together by means of molecular biological techniques.

As used herein, the term “regulatory element” refers to a genetic element which can control the expression of nucleic acid sequences. For example, a promoter is a regulatory element that facilitates the initiation of transcription of an operably linked coding region. Other regulatory elements are splicing signals, polyadenylation signals, termination signals, etc.

The term DNA “control sequences” refers collectively to regulatory elements such as promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need be present.

Transcriptional control signals in eukaryotes generally comprise “promoter” and “enhancer” elements. Promoters and enhancers consist of short arrays of DNA sequences that interact specifically with cellular proteins involved in transcription (Maniatis et al., Science 236:1237 (1987), incorporated herein by reference in its entirety). Promoter and enhancer elements have been isolated from a variety of eukaryotic sources including genes in yeast, insect and mammalian cells and viruses (analogous control sequences, i.e., promoters, are also found in prokaryotes). The selection of a particular promoter and enhancer depends on the recipient cell type. Some eukaryotic promoters and enhancers have a broad host range while others are functional in a limited subset of cell types (See e.g., Voss et al., Trends Biochem. Sci., 11:287 (1986); and Maniatis et al., supra, for reviews, incorporated herein by reference in their entirety). For example, the SV40 early gene enhancer is very active in a variety of cell types from many mammalian species and has been used to express proteins in a broad range of mammalian cells (Dijkema et al, EMBO J. 4:761 (1985), incorporated herein by reference in its entirety). Promoter and enhancer elements derived from the human elongation factor 1-alpha gene (Uetsuki et al., J. Biol. Chem., 264:5791 (1989); Kim et al., Gene 91:217 (1990); and Mizushima and Nagata, Nucl. Acids. Res., 18:5322 (1990)), the long terminal repeats of the Rous sarcoma virus (Gorman et al., Proc. Natl. Acad. Sci. U.S.A. 79:6777 (1982)), and the human cytomegalovirus (Boshart et al., Cell 41:521 (1985)) are also of utility for expression of proteins in diverse mammalian cell types, incorporated herein by reference in their entirety. Promoters and enhancers can be found naturally, alone or together. For example, retroviral long terminal repeats comprise both promoter and enhancer elements. Generally promoters and enhancers act independently of the gene being transcribed or translated. Thus, the enhancer and promoter used can be “endogenous,” “exogenous,” or “heterologous” with respect to the gene to which they are operably linked. An “endogenous” enhancer/promoter is one which is naturally linked with a given gene in the genome. An “exogenous” or “heterologous” enhancer or promoter is one which is placed in juxtaposition to a gene by means of genetic manipulation (i.e., molecular biological techniques) such that transcription of that gene is directed by the linked enhancer/promoter.

As used herein, the term “tissue specific” refers to regulatory elements or control sequences, such as a promoter, an enhancer, etc., wherein the expression of the nucleic acid sequence is substantially greater in a specific cell type(s) or tissue(s).

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York (1989), pp. 16.7-16.8, incorporated herein by reference in its entirety). A commonly used splice donor and acceptor site is the splice junction from the 16S RNA of SV40.

Transcription termination signals are generally found downstream of a polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which has been isolated from one gene and operably linked to the 3′ end of another gene. A commonly used heterologous poly A signal is the SV40 poly A signal. The SV40 poly A signal is contained on a 237 bp BamHI/BclI restriction fragment and directs both termination and polyadenylation (Sambrook et al., supra, at 16.6-16.7, incorporated herein by reference in its entirety).

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

As defined herein, a “therapeutically effective amount” or “therapeutic effective dose” is an amount or dose of a fusion protein, polypeptide, nucleic acid, lipid nanoparticle, liposome, AAV particle(s), or virion(s) capable of producing sufficient amounts of a desired protein to modulate the activity of the protein in a desired manner, thus providing a palliative tool for clinical intervention. In some embodiments, a therapeutically effective amount or dose of a transfected fusion protein, polypeptide, nucleic acid, AAV particle(s), or virion(s) as described herein is enough to confer suppression of a gene targeted by the fusion protein/gene therapy construct.

As used herein, the term “treat”, e.g., a disorder, means that a subject (e.g., a human) who has a disorder, is at risk of having a disorder, and/or experiences a symptom of a disorder, will, in an embodiment, suffer a less severe symptom and/or will recover faster, when a fusion molecule or a nucleic acid that encodes the fusion molecule, and/or a gRNA or a nucleic acid that encodes the gRNA, e.g., as described herein, is administered than if the fusion molecule or a nucleic acid that encodes the fusion molecule, and/or the gRNA or a nucleic acid that encodes the gRNA, were never administered.

### II. DNA Binding Proteins

In certain embodiments in the methods and compositions as defined herein according to the disclosure, the DNA binding protein (e.g. DNA targeting agent) comprises a (DNA) nuclease, such as a nuclease which can target DNA in a sequence specific manner or which can be directed or instructed to target DNA in a sequence specific manner, such as a CRISPR-Cas system, Zinc finger nuclease (ZFN), Transcription Activator-Like Effector Nuclease (TALEN), or meganuclease. In some embodiments, the DNA binding protein is a DNA nuclease derived from a CRISPR-Cas system.

### Transcription Activator-Like Effector Nuclease (TALEN) System

In certain embodiments, the nucleic acid binding protein is a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39: e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, each of which are incorporated by reference in their entirety.

By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In some embodiments, the nucleic acid is DNA.

As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers.

As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35) z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the disclosure, polypeptide monomers with an RVD of IG preferentially bind to T. Thus, the number and order of the polypeptide monomer repeats in the nucleic acid binding domain of a TALE determines its nucleic acid target specificity. In still further embodiments of the disclosure, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety. In certain embodiments, targeting is effected by a polynucleic acid binding TALEN fragment. In certain embodiments, the targeting domain comprises or consists of a catalytically inactive TALEN or nucleic acid binding fragment thereof.

### Zn-Finger Nuclease (ZFN) System

In certain embodiments, the targeting domain comprises or consists of a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794, 136, 6,824,978, 6,866,997, 6,933,113, 6,979,539, 7,013,219, 7,030,215, 7,220,719, 7,241,573, 7,241,574, 7,585,849, 7,595,376, 6,903, 185, and 6,479,626, each of which are incorporated by reference in their entirety. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. In certain embodiments, the targeting domain comprises or consists of a nucleic acid binding zinc finger nuclease or a nucleic acid binding fragment thereof. In certain embodiments, the nucleic acid binding (fragment of) a zinc finger nuclease is catalytically inactive.

### Meganuclease

In certain embodiments, the targeting domain comprises a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, each of which are incorporated by reference in their entirety. In certain embodiments, targeting is effected by a polynucleic acid binding meganuclease fragment. In certain embodiments, targeting is effected by a polynucleic acid binding catalytically inactive meganuclease (fragment). Accordingly in particular embodiments, the targeting domain comprises or consists of a nucleic acid binding meganuclease or a nucleic acid binding fragment thereof.

### CRISPR-Cas Systems

In some embodiments, the DNA binding protein and single guide RNA sequence of the present disclosure are derived from the CRISPR-Cas system. The present disclosure provides CRISPR/Cas9-based engineered systems for use in genome editing and treating genetic diseases. The CRISPR/Cas9-based engineered systems may be designed to target any gene (e.g. VEGF), including genes involved in a genetic disease, liver disease and dysregulation of cholesterol such as LDL. The present disclosure provides a CRISPR-Cas system comprising genetically engineered Cas proteins and/or guide RNAs with desired specificity and activity (e.g. reducing or eliminating expression of VEGF gene product). The CRISPR/Cas9-based systems may include a Cas9 protein, a mutated Cas9 protein or Cas9 fusion protein (e.g. DNMT3A-DNMT3L (3A3L)-dCas9-KRAB fusion molecule) and at least one sgRNA (e.g. VEGF sgRNA). The Cas9 fusion protein may, for example, include a domain that has a different activity from what is endogenous to Cas9 (e.g. DNMT3A, DNMT3L or KRAB).

In general, a Cas protein (used interchangeably herein with CRISPR protein, CRISPR enzyme, CRISPR-Cas protein, CRISPR-Cas enzyme, Cas, CRISPR effector, or Cas effector protein) and/or a guide sequence is a component of a CRISPR-Cas system. A CRISPR-Cas system or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, e.g. CRISPR RNA and transactivating (tracr) RNA or a single guide RNA (aka sgRNA; chimeric RNA)) or other sequences and transcripts from a CRISPR locus.

In general, a CRISPR system is characterized by elements that promote the formation of a CRISPR complex at the site of a target sequence (also referred to as a protospacer in the context of an endogenous CRISPR system). In an engineered system of the disclosure, the direct repeat may encompass naturally occurring sequences or non-naturally occurring sequences. The direct repeat of the disclosure is not limited to naturally occurring lengths and sequences. Furthermore, a direct repeat of the disclosure may include insertions of nucleotides such as an aptamer or sequences that bind to an adapter protein (for association with functional domains). In certain embodiments, one end of a direct repeat containing such as an insertion is roughly the first half of a short DR and the end is roughly the second half of the short DR.

In the context of formation of a CRISPR complex, “target sequence” or “target polynucleotides” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

In general, a guide sequence (or spacer sequence) may be any polynucleotide sequence having sufficient complementarity (e.g. perfect complementarity) with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 70%, 75%, 80%, 85%, 90%, 91%, 92%, 93%, 94%, 95%, 96%, 97%, 98%, 99%, or more.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance, a double mismatch is, the more cleavage efficiency is affected. Accordingly, by choosing mismatch positions along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

A CRISPR-Cas system or components thereof may be used for introducing one or more mutations in a target locus or nucleic acid sequence. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s).

Typically, in the context of an endogenous CRISPR-Cas system, formation of a CRISPR complex (comprising a guide sequence hybridized to a target sequence and complexed with one or more Cas proteins) results in cleavage in or near (e.g. within 1, 2, 3, 4, 5, 6, 7, 8, 9, 10, 20, 50, or more base pairs from) the target sequence, but may depend on for instance secondary structure, in particular in the case of RNA targets. In some cases, in the context of an endogenous CRISPR system, formation of a CRISPR complex (comprising a guide sequence hybridized to a target sequence and complexed with one or more Cas proteins) results in cleavage of one or both strands (if applicable) in or near (e.g. within 1, 2, 3, 4, 5, 6, 7, 8, 9, 10, 20, 50, or more base pairs from) the target sequence.

In some embodiments, the guide RNA (capable of guiding Cas to a target locus) may comprise (1) a guide sequence capable of hybridizing to a target locus (a polynucleotide target locus, such as an RNA target locus) in the eukaryotic cell; (2) a direct repeat (DR) sequence, which reside in a single RNA, i.e. an sgRNA (arranged in a 5′ to 3′ orientation) or crRNA.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant disclosure, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publication Nos. US 2014-0310830, US 2014-0287938 A1, US 2014-0273234 A1, US 2014-0273232 A1, US 2014-0273231 A1, US 2014-0256046 A1, US 2014-0248702 A1, US 2014-0242700 A1, US 2014-0242699 A1, US 2014-0242664 A1, US 2014-0234972 A1, US 2014-0227787 A1, US 2014-0189896 A1, US 2014-0186958, US 2014-0186919 A1, US 2014-0186843 A1, US 2014-0179770 A1 and US 2014-0179006 A1, US 2014-0170753; European Patents EP 2784162 B1 and EP 2771468 B1; European Patent Applications EP 2771468, EP 2764103, and EP 2784162; and PCT Patent Publications WO 2021/183807A1 (PCT/US2021/021973), WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), each of which are incorporated herein by reference in their entirety.

### Cas Proteins

The Cas protein (e.g., engineered Cas protein) may have a nuclease activity that is substantially the same (e.g., between 80% and 100%, between 90% and 100%, between 95% and 100%, between 98% and 100%, between 99% and 100%, between 99.9% and 100%, or about 100%) as a wildtype counterpart Cas protein. In certain cases, the engineered Cas protein has a nuclease activity that is higher than (e.g., at least 5%, at least 10%, at least 20%, at least 30%, at least 40%, at least 50%, at least 60%, at least 70%, at least 80%, or at least 90% higher than) a wildtype counterpart Cas protein.

Alternatively or additionally, the Cas protein (e.g., engineered Cas protein) may have a specificity at least 5%, at least 10%, at least 20%, at least 30%, at least 40%, at least 50%, at least 60%, at least 70%, at least 80%, or at least 90% higher than the wildtype counterpart Cas protein. In a particular example, the Cas protein (e.g., engineered Cas protein) may have a specificity at least 30% higher than the wildtype counterpart Cas protein. As used herein, the term “specificity” of a Cas may correspond to the number or percentage of on-target polynucleotide cleavage events relative to the number or percentage of all polynucleotide cleavage events, including on-target and off-target events. The activity and specificity of a Cas protein are consistent with those described in Hsu PD et al., DNA targeting specificity of RNA-guided Cas9 nucleases, Nat Biotechnol. 2013 September; 31 (9): 827-832; and Slaymaker IM, et al., Rationally engineered Cas9 nucleases with improved specificity, Science. 2016 Jan. 1; 351 (6268): 84-88, which also describe examples of methods for detecting the activity and specificity of Cas proteins, and are incorporated herein by reference in their entireties, and are detailed elsewhere herein.

In some embodiments, the Cas protein (e.g., its RuvC domain) may slide one base upstream (with respect to the PAM), and produce a staggered cut, which may be filled and lead to duplication of a single base (i.e., +1 insertion). An example of a +1 insertion position is described in Zuo, Z., and Liu, J. (2016). Cas9-catalyzed DNA Cleavage Generates Staggered Ends: Evidence from Molecular Dynamics Simulations. Scientific Reports 6, 37584. In some embodiments, the engineered Cas protein has a +1 insertion frequency different from the wildtype counterpart Cas protein. For example, the +1 insertion frequency when a guanine is present in the −2 position with respect a PAM is higher than the +1 insertion frequency when a thymidine, a cytidine, or a adenine is present in the −2 position with respect the PAM. In some cases, the +1 insertions depend on host machinery in human cells. In some examples, the Cas protein may generate a staggered cut. The staggered cut may be a 1-bp or 1-nucleotide 5′ overhang. The staggered cut may be a 1-bp or 1-nucleotide 3′ overhang.

The nucleic acid molecule encoding a Cas may be codon optimized. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in tum believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In some embodiments, the Cas proteins may have nucleic acid cleavage activity. The Cas proteins may have RNA binding and DNA cleaving function. In some embodiments, Cas may direct cleavage of one or two nucleic acid strands at the location of or near a target sequence, such as within the target sequence and/or within the complement of the target sequence or at sequences associated with the target sequence, e.g., within about 1, 2, 3, 4, 5, 6, 7, 8, 9, 10, 15, 20, 25, 50, 100, 200, 500, or more base pairs from the first or last nucleotide of a target sequence. In some embodiments, the Cas protein may direct more than one cleavage (such as one, two, three, four, five, or more cleavages) of one or two strands within the target sequence and/or within the complement of the target sequence or at sequences associated with the target sequence and/or within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt, i.e., generating blunt ends. In some embodiments, the cleavage may be staggered, i.e., generating sticky ends.

In some embodiments, a vector encodes a nucleic acid-targeting Cas protein that may be mutated with respect to a corresponding wild-type enzyme such that the mutated nucleic acid-targeting Cas protein lacks the ability to cleave one or two strands of a target polynucleotide containing a target sequence, e.g., alteration or mutation in a HNH domain to produce a mutated Cas substantially lacking all DNA cleavage activity, e.g., the DNA cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “derived” with reference to an enzyme means that the derived enzyme is largely based, in the sense of having a high degree of sequence homology with, a wildtype enzyme, but that it has been mutated (modified) in some way as known in the art or as described herein.

Typically, in the context of an endogenous nucleic acid-targeting system, formation of a nucleic acid-targeting complex (comprising a guide RNA or crRNA hybridized to a target sequence and complexed with one or more nucleic acid-targeting effector proteins) results in cleavage of DNA strand(s) in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest).

It will be appreciated that the effector protein is based on or derived from an enzyme, so the term “effector protein” certainly includes “enzyme” in some embodiments. However, it will also be appreciated that the effector protein may, as required in some embodiments, have DNA or RNA binding, but not necessarily cutting or nicking, activity, including a dead-Cas protein function.

In some embodiments, a Cas protein may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the CRISPR effector protein may be a part of a Light Inducible Transcriptional Effector (LITE) to direct changes in transcriptional activity in a sequence-specific manner. The components of a light may include a CRISPR effector protein, a light-responsive cytochrome heterodimer (e.g. from Arabidopsis thaliana), and a transcriptional activation/repression domain. Further examples of inducible DNA binding proteins and methods for their use are provided in U.S. 61/736,465 and U.S. 61/721,283, and WO 2014018423 A2 which are hereby incorporated by reference in their entirety.

In some embodiments, a mutated Cas may have one or more mutations resulting in reduced off-target effects, e.g., improved CRISPR enzymes for use in effecting modifications to target loci but which reduce or eliminate activity towards off-targets, such as when complexed to guide RNAs, as well as improved CRISPR enzymes for increasing the activity of CRISPR enzymes, such as when complexed with guide RNAs. It is to be understood that mutated enzymes as described herein below may be used in any of the methods according to the disclosure as described herein elsewhere. Any of the methods, products, compositions and uses as described herein elsewhere are equally applicable with the mutated CRISPR enzymes as further detailed below.

The methods and mutations which can be employed in various combinations to increase or decrease activity and/or specificity of on-target vs. off-target activity, or increase or decrease binding and/or specificity of on-target vs. off-target binding, can be used to compensate or enhance mutations or modifications made to promote other effects. Such mutations or modifications made to promote other effects in include mutations or modification to the Cas and/or mutation or modification made to a guide RNA. The methods and mutations of the disclosure are used to modulate Cas nuclease activity and/or binding with chemically modified guide RNAs.

In certain embodiments, the catalytic activity of the Cas protein of the disclosure is altered or modified. It is to be understood that mutated Cas has an altered or modified catalytic activity if the catalytic activity is different than the catalytic activity of the corresponding wild type Cas protein (e.g., unmutated Cas protein). Catalytic activity can be determined by means known in the art. By means of example, and without limitation, catalytic activity can be determined in vitro or in vivo by determination of indel percentage (for instance after a given time, or at a given dose). In certain embodiments, catalytic activity is increased. In certain embodiments, catalytic activity is increased by at least 5%, at least 10%, at least 20%, at least 30%, at least 40%, at least 50%, at least 60%, at least 70%, at least 80%, at least 90%, or at least 100%. In certain embodiments, catalytic activity is decreased. In certain embodiments, catalytic activity is decreased by at least 5%, preferably at least 10%, more preferably at least 20%, such as at least 30%, at least 40%, at least 50%, at least 60%, at least 70%, at least 80%, at least 90%, or (substantially) 100%. The one or more mutations herein may inactivate the catalytic activity, which may substantially decrease all catalytic activity, decrease activity to below detectable levels, or decrease to no measurable catalytic activity.

One or more characteristics of the engineered Cas protein may be different from a corresponding wildtype Cas protein. Examples of such characteristics include catalytic activity, gRNA binding, specificity of the Cas protein (e.g., specificity of editing a defined target), stability of the Cas protein, off-target binding, target binding, protease activity, nickase activity, PFS recognition. In some examples, an engineered Cas protein may comprise one or more mutations of the corresponding wild type Cas protein. In some embodiments, the catalytic activity of the engineered Cas protein is increased as compared to a corresponding wildtype Cas protein. In some embodiments, the catalytic activity of the engineered Cas protein is decreased as compared to a corresponding wildtype Cas protein. In some embodiments, the gRNA binding of the engineered Cas protein is increased as compared to a corresponding wildtype Cas protein. In some embodiments, the gRNA binding of the engineered Cas protein is decreased as compared to a corresponding wildtype Cas protein. In some embodiments, the specificity of the Cas protein is increased as compared to a corresponding wildtype Cas protein. In some embodiments, the specificity of the Cas protein is decreased as compared to a corresponding wildtype Cas protein. In some embodiments, the stability of the Cas protein is increased as compared to a corresponding wildtype Cas protein. In some embodiments, the stability of the Cas protein is decreased as compared to a corresponding wildtype Cas protein. In some embodiments, the engineered Cas protein further comprises one or more mutations which inactivate catalytic activity. In some embodiments, the off-target binding of the Cas protein is increased as compared to a corresponding wildtype Cas protein. In some embodiments, the off-target binding of the Cas protein is decreased as compared to a corresponding wildtype Cas protein. In some embodiments, the target binding of the Cas protein is increased as compared to a corresponding wildtype Cas protein. In some embodiments, the target binding of the Cas protein is decreased as compared to a corresponding wildtype Cas protein. In some embodiments, the engineered Cas protein has a higher protease activity or polynucleotide-binding capability compared with a corresponding wildtype Cas protein. In some embodiments, the PFS recognition is altered as compared to a corresponding wildtype Cas protein.

### Examples of Cas Proteins

Examples of Cas proteins include those of Class I (e.g., Type I, Type III, and Type IV) and Class 2 (e.g., Type II, Type V, and Type VI) Cas proteins, e.g., Cas9, Cas12 (e.g., Cas12a, Cas12b, Cas12c, Cas12d), Cas13 (e.g., Cas13a, Cas13b, Cas13c, Cas13d,), CasX, CasY, Cas14, variants thereof (e.g., mutated forms, truncated forms), homologs thereof, and orthologs thereof. The terms “ortholog” and “homolog” are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Class 2 Cas Proteins

In some embodiments, the Cas protein is a class 2 Cas protein, i.e., a Cas protein of a class 2 CRISPR-Cas system. A class 2 CRISPR-Cas system may be of a subtype, e.g., Type II-A, Type II-B, Type II-C, Type V-A, Type V-B, Type V-C, or Type V-U. In some embodiments, the Cas protein is Cas9, Cas12a, Cas12b, Cas12c, or Cas12d. In some embodiments, Cas9 may be SpCas9, SaCas9, StCas9 and other Cas9 orthologs. Cas12 may be Cas12a, Cas12b, and Cas12c, including FnCas12a, or homology or orthologs thereof. The definition and exemplary members of the CRISPR-Cas system include those described in Kira S. Makarova and Eugene V. Koonin, Annotation and Classification of CRISPR-Cas systems, Methods Mol Biol. 2015; 1311:47-75; and Sergey Shmakov et al., Diversity and evolution of class 2 CRISPR-Cas systems, Nat Rev Microbial. 2017 March; 15 (3): 169-182:.

### Cas Protein Linkers

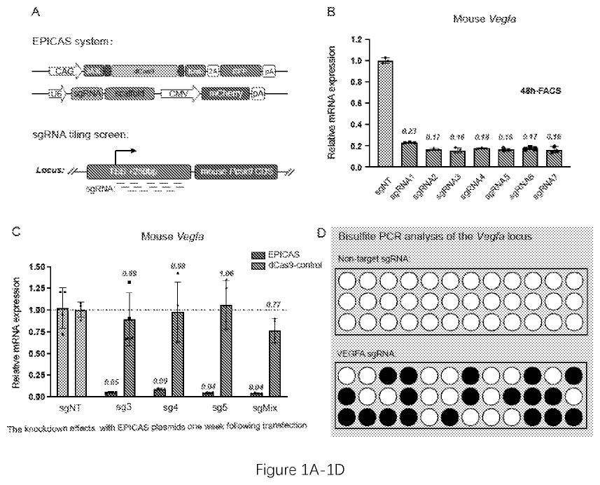

In some examples, the Cas protein comprises at least one RuvC domain and at least one HNH domain. The Cas protein may further comprise a first and a second linker domain connecting the RuvC domain and the HNH domain. The first linker (L1) and second linker (L2) connecting the HNH and RuvC domains in Cas9 are described in studies by Nishimasu, H. et al. “Crystal structure of Cas9 in complex with guide RNA and target RNA” Cell 156 (Feb. 27, 2014): 935-949 and Ribeiro, L. et al. (2018) “Protein engineering strategies to expand CRISPR-Cas9 applications” International Journal of Genomics Volume 2018, Article ID 1652567 (doi.org/10.1155/2018/1652567). FIG. 1 of Ribeiro shows the overall organization, structure and function of Cas9, incorporated specifically herein by reference. Specifically, FIG. 1A shows a schematic representation of the domain organization of SpCas9 indicating the genetic architecture of the HNH and RuvC domains including the linkers L1 (spanning amino acids 765-780) and L2 (spanning amino acids 906-918) as described herein.

Similarly, the domain organization of Staphylococcus aureus Cas9 (SaCas9) can be utilized when referencing the first and second linker domains. In an aspect, the Linker 1 domain region spans residues 481-519, and connects the RuvC-II domain to the HNH domain in SaCas9. In some embodiments, Linker 2 region spans residues 629-649, and connects the RuvC-III domain and the HNH domain of SaCas9. Accordingly, the first and/or second linker domain may be mutated in a Cas9 ortholog, and reference may be made to amino acid residues corresponding to the amino acids of a wild-type SaCas9. See, Nishimasu, Cell. 2015 Aug. 27; 162 (5): 1113-1126; doi: 10.1016/j.cell.2015.08.007, incorporated by reference herein. In particular, FIG. 1, S1-S3 of Nishimasu detail domain organization of Cas9 proteins, and are incorporated specifically by reference herein for their teachings.

The first and second linker may comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more amino acids. The first and second linker may correspond to wild-type linkers. In some aspects, the first and second linkers may comprise one or more mutations in the first and/or second linker. In an aspect the first and/or second linker comprise one or more mutations that improve specificity of the Cas9 protein.

In some embodiments, the linkers, L1 and L2, connecting the HNH and RuvC domains of Cas9 contain the wild-type amino acid sequences. In some embodiments, the linkers connecting the HNH and RuvC domains contain mutations in one or more amino acids. In an example embodiment, the first linker (L1) contains the mutation corresponding to amino acid T7691 of SpCas9 and/or the second linker (L2) contains the mutation corresponding to amino acid G915M of SpCas9. In an example embodiment, one or more linker mutations, e.g., T7691 and G915M, confer improved specificity upon the Cas9 protein.

In one embodiment, one or mutations in the first and second linker may be combined with one or more mutations in other portions of the Cas9 protein for further improved specificity and/or retention of activity that is substantially equivalent to a wild-type Cas9 protein, as described herein.

In one embodiment, mutations in the linker and/or additional mutations within the Cas protein can be identified utilizing the methods detailed herein that enhance/improve specificity and substantially retain wild-type activity to the wild-type Cas9.

Class 2, Type II Cas Proteins (e.g. Cas9)

In some embodiments, the Cas protein may be a Cas protein of a Class 2, Type II CRISPR-Cas system (a Type II Cas protein). In some embodiments, the Cas protein may be a class 2 Type II Cas protein, e.g., Cas9. In some embodiments, the CRISPR/Cas9-based system may include a Cas9 protein or a fragment thereof, a Cas9 fusion protein, a nucleic acid encoding a Cas9 protein or a fragment thereof, or a nucleic acid encoding a Cas9 fusion protein. By “Cas9 (CRISPR associated protein 9)” is meant a polypeptide or fragment thereof having at least about 85% amino acid identity to NCBI Accession No. NP_269215 and having RNA binding activity, DNA binding activity, and/or DNA cleavage activity (e.g., endonuclease or nickase activity). “Cas9 function” can be defined by any of a number of assays including, but not limited to, fluorescence polarization-based nucleic acid bind assays, fluorescence polarization-based strand invasion assays, transcription assays, EGFP disruption assays, DNA cleavage assays, and/or Surveyor assays, for example, as described herein. By “Cas 9 nucleic acid molecule” is meant a polynucleotide encoding a Cas9 polypeptide or fragment thereof. An exemplary Cas9 nucleic acid molecule sequence is provided at genome sequence No. NC_002737. In some embodiments, disclosed herein are inhibitors of Cas9, e.g., naturally occurring Cas9 in S. pyogenes (SpCas9) or S. aureus (SaCas9), or variants thereof. Cas9 recognizes foreign DNA using Protospacer Adjacent Motif (PAM) sequence and the base pairing of the target DNA by the guide RNA (gRNA). The relative ease of inducing targeted strand breaks at any genomic loci by Cas9 has enabled efficient genome editing in multiple cell types and organisms. Cas9 derivatives can also be used as transcriptional activators/repressors.

In some cases, the CRISPR-Cas protein is Cas9 or a variant thereof. In some examples, Cas9 may be wildtype Cas9 including any naturally occurring bacterial Cas9. Cas9 orthologs typically share the general organization of 3-4 RuvC domains and an HNH domain. The 5′ most RuvC domain cleaves the non-complementary strand, and the HNH domain cleaves the complementary strand. All notations are in reference to the guide sequence. The catalytic residue in the 5′ RuvC domain is identified through homology comparison of the Cas9 of interest with other Cas9 orthologs (from S. pyogenes type II CRISPR locus, S. thermophilus CRISPR locus 1, S. thermophilus CRISPR locus 3, and Franciscilla novicida type II CRISPR locus), and the conserved Asp residue (D10) is mutated to alanine to convert Cas9 into a complementary-strand nicking enzyme. Accordingly, the Cas enzyme can be wildtype Cas9 including any naturally occurring bacterial Cas9. The CRISPR, Cas or Cas9 enzyme can be codon optimized, or a modified version, including any chimaeras, mutants, homologs or orthologs. In an additional aspect of the disclosure, a Cas9 enzyme may comprise one or more mutations and may be used as a generic DNA binding protein with or without fusion to a functional domain.

The mutations may be artificially introduced mutations or gain-of-function or loss-of-function mutations. In some embodiments, the transcriptional activation domain may be VP64. In some embodiments, the transcriptional repressor domain may be KRAB or SID4X. Other aspects of the disclosure relate to the mutated Cas9 enzyme being fused to domains which include but are not limited to a nuclease, a transcriptional activator, repressor, a recombinase, a transposase, a histone remodeler, a demethylase, a DNA methyltransferase, a cryptochrome, a light inducible/controllable domain or a chemically inducible/controllable domain. The disclosure can involve sgRNAs or tracrRNAs or guide or chimeric guide sequences that allow for enhancing performance of these RNAs in cells. This type II CRISPR enzyme may be any Cas enzyme. In some cases, the Cas9 enzyme is from, or is derived from, SpCas9 or SaCas9. As used herein, the term “derived” with reference to an enzyme means that the derived enzyme is largely based, in the sense of having a high degree of sequence homology with, a wildtype enzyme, but that it has been mutated (modified) in some way as known in the art or as described herein. In an example the mutation may comprise one or more mutations in a first linker domain, a second linker domain, and/or other portions of the protein. The high degree of sequence homology may comprise at least 80%, 85%, 86%, 87%, 88%, 89%, 90%, 91%, 92%, 93%, 94%, 95%, 96%, 97%, 98%, 99% or more relative to a wildtype enzyme.

A Cas enzyme may be identified Cas9 as this can refer to the general class of enzymes that share homology to the biggest nuclease with multiple nuclease domains from the type II CRISPR system. In some cases, the Cas9 enzyme is from, or is derived from, SpCas9 (S. pyogenes Cas9) or saCas9 (S. aureus Cas9). “StCas9” refers to wildtype Cas9 from S. thermophilus (UniProt ID: G3ECR1). Similarly, “SpCas9” refers to wildtype Cas9 from S. pyogenes (UniProt ID: Q99ZW2). As used herein, the term “derived” with reference to an enzyme means that the derived enzyme is largely based, in the sense of having a high degree of sequence homology with, a wildtype enzyme, but that it has been mutated (modified) in some way as known in the art or as described herein. It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes.

In particular embodiments, the effector protein is a Cas9 effector protein from or originated from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacte, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus, Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter.

In some embodiments, the Cas9 protein is from or originated from an organism selected from S. mutans, S. agalactiae, S. equisimilis, S. sanguinis, S. pneumonia, C. jejuni, C. coli; N salsuginis, N tergarcus; S. auricularis, S. carnosus; N meningitides, N gonorrhoeae, L. monocytogenes, L. ivanovii; C. botulinum, C. difficile, C. tetani, or C. sordellii, Francisella tularensis 1, Francisella tularensis subsp. novicida, Prevotella albensis, Lachnospiraceae bacterium MC2017 1, Butyrivibrio proteoclasticus, Peregrinibacteria bacterium GW2011 GWA2_33 10, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. In some embodiments, Cas9 effector protein from an organism from or originated from Streptococcus pyogenes, Staphylococcus aureus, or Streptococcus thermophilus Cas9.

In a more preferred embodiment, the Cas9 protein is derived from a bacterial species selected from Streptococcus pyogenes, Staphylococcus aureus, or Streptococcus thermophilus Cas9. In certain embodiments, the Cas9 is derived from a bacterial species selected from Francisella tularensis 1, Prevotella albensis, Lachnospiraceae bacterium MC20171, Butyrivibrio proteoclasticus, Peregrinibacteria bacterium GW2011 GWA2 33 JO, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cas9 protein is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

Cas9 enzymes include but are not limited to S. pyogenes serotype M1 (UniProt ID: Q99ZW2), S. aureus Cas9 (UniProt ID: J7RUA5), Eubacterium ventriosum Cas9 (UniProt ID: A5Z395), Azospirillum (strain B510) Cas9 (UniProt ID: D3NT09), Gluconacetobacter diazotrophicus (strain ATCC 49037) Cas9 (UnitProt ID: A9HKP2), Nisseria cinerea Cas9 (UniProt ID: DOW2Z9), Roseburia intestinalis Cas9 (UniProt ID: C7G697), Parvibaculum lavamentivorans (strain DS-1) Cas9 (UniProt ID: A7HP89), Nitratifractor salsuginis (strain DSM 16511) Cas9 (UniProt ID: E6WZS9), Campylobacter lari Cas9 (UniProt ID: G1UFN3).

Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archaea, Mole Cell 2010 January 15; 37 (1): 7. The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA: tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. A pre-crRNA array consisting of a single spacer flanked by two direct repeats (DRs) is also encompassed by the term “tracr-mate sequences”). In certain embodiments, Cas9 may be constitutively present or inducibly present or conditionally present or administered or delivered. Cas9 optimization may be used to enhance function or to develop new functions. One can generate chimeric Cas9 proteins and Cas9 may be used as a generic DNA binding protein. The structural information provided for Cas9 may be used to further engineer and optimize the CRISPR-Cas system and this may be extrapolated to interrogate structure-function relationships in other CRISPR enzyme systems as well, particularly structure-function relationships in other Type II CRISPR enzymes or Cas9 orthologs. The crystal structure information (described in U.S. provisional applications 61/915,251 filed Dec. 12, 2013, 61/930,214 filed on Jan. 22, 2014, 61/980,012 filed Apr. 15, 2014; and Nishimasu et al, “Crystal Structure of Cas9 in Complex with Guide RNA and Target DNA,” Cell 156 (5): 935-949, DOI: ttp://dx.doi.org/10.1016/j.cell.2014.02.001 (2014), each and all of which are incorporated herein by reference) provides structural information to truncate and create modular or multi-part CRISPR enzymes which may be incorporated into inducible CRISPR-Cas systems. In particular, structural information is provided for S. pyogenes Cas9 (SpCas9) and this may be extrapolated to other Cas9 orthologs or other Type II CRISPR enzymes. The Cas9 gene is found in several diverse bacterial genomes, typically in the same locus with cas1, cas2, and cas4 genes and a CRISPR cassette. Furthermore, the Cas9 protein contains a readily identifiable C-terminal region that is homologous to the transposon ORF-B and includes an active RuvC-like nuclease, an arginine-rich region.

dCas9

The Cas9 protein may be mutated so that the nuclease activity is inactivated. An inactivated Cas9 protein from S. pyogenes (iCas9, also referred to as “dCas9”) with no endonuclease activity has been recently targeted to genes in bacteria, yeast, and human cells by gRNA to silence gene expression through steric hindrance. As used herein, a “dCas molecule” may refer to a dCas protein, or a fragment thereof. As used herein, a “dCas9 molecule” may refer to a dCas9 protein, or a fragment thereof. As used herein, the terms “iCas” and “dCas” are used interchangeably and refer to a catalytically inactive CRISPR associated protein. In one embodiment, the dCas molecule comprises one or more mutations in a DNA-cleavage domain. In one embodiment, the dCas molecule comprises one or more mutations in the RuvC or domain. In one embodiment, the dCas molecule comprises one or more mutations in both the RuvC and HNH domain. In one embodiment, the dCas molecule is a fragment of a wild-type Cas molecule. In one embodiment, the dCas molecule comprises a functional domain from a wild-type Cas molecule, wherein the functional domain is chosen from a Reel domain, a bridge helix domain, or a PAM interacting domain. In one embodiment, the nuclease activity of the dCas molecule is reduced by at least 40%, 45%, 50%, 55%, 60%, 65%, 70%, 75%, 80%, 85%, 90%, 95%, or 99% compared to that of a corresponding wild type Cas molecule.

Suitable dCas molecule can be derived from a wild type Cas molecule. The Cas molecule can be from a type I, type II, or type III CRISPR-Cas systems. In one embodiment, suitable dCas molecules can be derived from a Cas1, Cas2, Cas3, Cas4, Cas5, Cas6, Cas7, Cas8, Cas9, or Cas10 molecule. In one embodiment, the dCas molecule is derived from a Cas9 molecule. The dCas9 molecule can be obtained, for example, by introducing point mutations (e.g., substitutions, deletions, or additions) in the Cas9 molecule at the DNA-cleavage domain, e.g., the nuclease domain, e.g., the RuvC and/or HNH domain. See, e.g., Jinek et al., Science (2012) 337:816-21, incorporated by reference herein in its entirety. For example, introducing two point mutations in the RuvC and HNH domains reduces the Cas9 nuclease activity while retaining the Cas9 sgRNA and DNA binding activity. In one embodiment, the two point mutations within the RuvC and HNH active sites are D10A and H840A mutations of the S. pyogenes Cas9 molecule. Alternatively, D10 and H840 of the S. pyogenes Cas9 molecule can be deleted to abolish the Cas9 nuclease activity while retaining its sgRNA and DNA binding activity. In one embodiment, the two point mutations within the RuvC and HNH active sites are D10A and N580A mutations of the S. pyogenes Cas9 molecule.

In various embodiments, the present disclosure involves a dCas molecule or a variant or a mutant of any of the variants thereof. All variants and mutants of dCas9 can be used in a method, composition, or kit disclosed herein, including but not limited to those derived from SpCas9 (Cas9 isolated from Streptococcus pyogenes), SaCas9 (Cas9 isolated from Staphylococcus aureus), StCas9 (Cas9 isolated from Streptococcus thermophilus), NmCas9 (Cas9 isolated from Neisseria meningitidis), FnCas9 (Cas9 isolated from Francisella novicida), CjCas9 (Cas9 isolated from Campylobacter jejuni), ScCas9 (Cas9 isolated from Streptococcus canis), and any variants and mutant forms of the Cas9 listed above, such as high-fidelity Cas9 (Kleinstiver et al., Nature. 2016 Jan. 28) and enhanced SpCas9 (Slaymaker et al., Sciences. 2016 Jan. 1). This list is only to provide several exemplary options and is not exclusive.

In one embodiment, the dCas molecule is a Streptococcus pyogenes dCas9 molecule comprising a mutation at D10 and/or H840, numbered according to SEQ ID NO: 1. In one embodiment, the dCas molecule is a Streptococcus pyogenes dCas9 molecule comprising D10A and/or H840A mutations, numbered according to SEQ ID NO: 1.

Streptococcus pyogenes dCas9
(SEQ ID NO: 1)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In one embodiment, the dCas9 molecule is a Staphylococcus aureus dCas9 molecule comprising the amino acid sequence of SEQ ID NO: 2 or 3, a sequence substantially identical (e.g., at least 80%, 85%, 90%, 91%, 92%, 93%, 94%, 95%, 96%, 97%, 98%, 99% or higher in sequence identity) to SEQ ID NO: 2 or 3, or a sequence having one, two, three, four, five or more changes, e.g., amino acid substitutions, insertions, or deletions, relative to SEQ ID NO: 2 or 3, or any fragment thereof.

Similar mutations can also apply to any other naturally-occurring Cas9 (e.g., Cas9 from other species) or engineered Cas9 molecules. In certain embodiments, the dCas9 molecule comprises a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B 510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 1651 1) dCas9 molecule, a Campylobacter lari (strain CF89-12) dCas9 molecule, a Streptococcus thermophilus (strain LMD-9) dCas9 molecule, or fragment thereof.

In certain embodiments, the present disclosure provides a vector comprising a nucleotide encoding a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 1651 1) dCas9 molecule, a Campylobacter lari (strain CF89-12) dCas9 molecule, a Streptococcus thermophilus (strain LMD-9) dCas9 molecule, or fragment thereof.

Exemplary dCas9 proteins include but are not limited to those listed in Table 1.

TABLE 1
Exemplary dCas9 proteins

| | | SEQ ID |
|---|---|---|
| Description | Sequence | NO: |
| Staphylococcus | MKRNYILGLAIGITSVGYGIIDYETRDVIDAGVRLFKEANVENNEGR | 2 |
| aureus dCas9 | RSKRGARRLKRRRRHRIQRVKKLLFDYNLLTDHSELSGINPYEARVK | |
| (Mutant: | GLSQKLSEEEFSAALLHLAKRRGVHNVNEVEEDTGNELSTKEQISRN | |
| D10A and H557A) | SKALEEKYVAELQLERLKKDGEVRGSINRFKTSDYVKEAKQLLKVQK | |
| | AYHQLDQSFIDTYIDLLETRRTYYEGPGEGSPFGWKDIKEWYEMLMG | |
| | HCTYFPEELRSVKYAYNADLYNALNDLNNLVITRDENEKLEYYEKFQ | |
| | IIENVFKQKKKPTLKQIAKEILVNEEDIKGYRVTSTGKPEFTNLKVY | |
| | HDIKDITARKEIIENAELLDQIAKILTIYQSSEDIQEELTNLNSELT | |
| | QEEIEQISNLKGYTGTHNLSLKAINLILDELWHTNDNQIAIFNRLKL | |
| | VPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENA | |
| | KYLIEKIKLHDMQEGKCLYSLEAIPLEDLLNNPFNYEVDAIIPRSVS | |
| | FDNSFNNKVLVKQEENSKKGNRTPFQYLSSSDSKISYETFKKHILNL | |
| | AKGKGRISKTKKEYLLEERDINRFSVQKDFINRNLVDTRYATRGLMN | |
| | LLRSYFRVNNLDVKVKSINGGFTSFLRRKWKFKKERNKGYKHHAEDA | |
| | LIIANADFIFKEWKKLDKAKKVMENQMFEEKQAESMPEIETEQEYKE | |
| | IFITPHQIKHIKDFKDYKYSHRVDKKPNRELINDTLYSTRKDDKGNT | |
| | LIVNNLNGLYDKDNDKLKKLINKSPEKLLMYHHDPQTYQKLKLIMEQ | |
| | YGDEKNPLYKYYEETGNYLTKYSKKDNGPVIKKIKYYGNKLNAHLDI | |
| | TDDYPNSRNKVVKLSLKPYRFDVYLDNGVYKFVTVKNLDVIKKENYY | |
| | EVNSKCYEEAKKLKKISNQAEFIASFYNNDLIKINGELYRVIGVNND | |
| | LLNRIEVNMIDITYREYLENMNDKRPPRIIKTIASKTQSIKKYSTDI | |
| | LGNLYEVKSKKHPQIIKKG | |
| Staphylococcus | MKRNYILGLAIGITSVGYGIIDYETRDVIDAGVRLFKEANVENNEGR | 3 |
| aureus dCas9 | RSKRGARRLKRRRRHRIQRVKKLLFDYNLLTDHSELSGINPYEARVK | |
| (Mutant: | GLSQKLSEEEFSAALLHLAKRRGVHNVNEVEEDTGNELSTKEQISRN | |
| D10A and N580A) | SKALEEKYVAELQLERLKKDGEVRGSINRFKTSDYVKEAKQLLKVQK | |
| | AYHQLDQSFIDTYIDLLETRRTYYEGPGEGSPFGWKDIKEWYEMLMG | |
| | HCTYFPEELRSVKYAYNADLYNALNDLNNLVITRDENEKLEYYEKFQ | |
| | IIENVFKQKKKPTLKQIAKEILVNEEDIKGYRVTSTGKPEFTNLKVY | |
| | HDIKDITARKEIIENAELLDQIAKILTIYQSSEDIQEELTNLNSELT | |
| | QEEIEQISNLKGYTGTHNLSLKAINLILDELWHTNDNQIAIFNRLKL | |
| | VPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENA | |
| | KYLIEKIKLHDMQEGKCLYSLEAIPLEDLLNNPFNYEVHAIIPRSVS | |
| | FDNSFNNKVLVKQEEASKKGNRTPFQYLSSSDSKISYETFKKHILNL | |
| | AKGKGRISKTKKEYLLEERDINRFSVQKDFINRNLVDTRYATRGLMN | |
| | LLRSYFRVNNLDVKVKSINGGFTSFLRRKWKFKKERNKGYKHHAEDA | |
| | LIIANADFIFKEWKKLDKAKKVMENQMFEEKQAESMPEIETEQEYKE | |
| | IFITPHQIKHIKDFKDYKYSHRVDKKPNRELINDTLYSTRKDDKGNT | |
| | LIVNNLNGLYDKDNDKLKKLINKSPEKLLMYHHDPQTYQKLKLIMEQ | |
| | YGDEKNPLYKYYEETGNYLTKYSKKDNGPVIKKIKYYGNKLNAHLDI | |
| | TDDYPNSRNKVVKLSLKPYRFDVYLDNGVYKFVTVKNLDVIKKENYY | |
| | EVNSKCYEEAKKLKKISNQAEFIASFYNNDLIKINGELYRVIGVNND | |
| | LLNRIEVNMIDITYREYLENMNDKRPPRIIKTIASKTQSIKKYSTDI | |
| | LGNLYEVKSKKHPQIIKKG | |
| Staphylococcus | MKRNYILGLAIGITSVGYGIIDYETRDVIDAGVRLFKEANVENNEGR | 4 |
| aureus dCas9 | RSKRGARRLKRRRRHRIQRVKKLLFDYNLLTDHSELSGINPYEARVK | |
| (D10A, H557A, | GLSQKLSEEEFSAALLHLAKRRGVHNVNEVEEDTGNELSTKEQISRN | |
| N580A) | SKALEEKYVAELQLERLKKDGEVRGSINRFKTSDYVKEAKQLLKVQK | |
| | AYHQLDQSFIDTYIDLLETRRTYYEGPGEGSPFGWKDIKEWYEMLMG | |
| | HCTYFPEELRSVKYAYNADLYNALNDLNNLVITRDENEKLEYYEKFQ | |
| | IIENVFKQKKKPTLKQIAKEILVNEEDIKGYRVTSTGKPEFTNLKVY | |
| | HDIKDITARKEIIENAELLDQIAKILTIYQSSEDIQEELTNLNSELT | |
| | QEEIEQISNLKGYTGTHNLSLKAINLILDELWHTNDNQIAIFNRLKL | |
| | VPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENA | |
| | KYLIEKIKLHDMQEGKCLYSLEAIPLEDLLNNPFNYEVHAIIPRSVS | |
| | FDNSFNNKVLVKQEEASKKGNRTPFQYLSSSDSKISYETFKKHILNL | |
| | AKGKGRISKTKKEYLLEERDINRFSVQKDFINRNLVDTRYATRGLMN | |
| | LLRSYFRVNNLDVKVKSINGGFTSFLRRKWKFKKERNKGYKHHAEDA | |
| | LIIANADFIFKEWKKLDKAKKVMENQMFEEKQAESMPEIETEQEYKE | |
| | IFITPHQIKHIKDFKDYKYSHRVDKKPNRELINDTLYSTRKDDKGNT | |
| | LIVNNLNGLYDKDNDKLKKLINKSPEKLLMYHHDPQTYQKLKLIMEQ | |
| | YGDEKNPLYKYYEETGNYLTKYSKKDNGPVIKKIKYYGNKLNAHLDI | |
| | TDDYPNSRNKVVKLSLKPYRFDVYLDNGVYKFVTVKNLDVIKKENYY | |
| | EVNSKCYEEAKKLKKISNQAEFIASFYNNDLIKINGELYRVIGVNND | |
| | LLNRIEVNMIDITYREYLENMNDKRPPRIIKTIASKTQSIKKYSTDI | |
| | LGNLYEVKSKKHPQIIKKG | |
| Campylobacter | MARILAFAIGISSIGWAFSENDELKDCGVRIFTKVENPKTGESLALP | 5 |
| jejuni | RRLARSARKRLARRKARLNHLKHLIANEFKLNYEDYQSFDESLAKAY | |
| dCas9 | KGSLISPYELRFRALNELLSKQDFARVILHIAKRRGYDDIKNSDDKE | |
| (D8A, H559A) | KGAILKAIKQNEEKLANYQSVGEYLYKEYFQKFKENSKEFTNVRNKK | |
| | ESYERCIAQSFLKDELKLIFKKQREFGFSFSKKFEEEVLSVAFYKRA | |
| | LKDFSHLVGNCSFFTDEKRAPKNSPLAFMFVALTRIINLLNNLKNTE | |
| | GILYTKDDLNALLNEVLKNGTLTYKQTKKLLGLSDDYEFKGEKGTYF | |
| | IEFKKYKEFIKALGEHNLSQDDLNEIAKDITLIKDEIKLKKALAKYD | |
| | LNQNQIDSLSKLEFKDHLNISFKALKLVTPLMLEGKKYDEACNELNL | |
| | KVAINEDKKDFLPAFNETYYKDEVTNPVVLRAIKEYRKVLNALLKKY | |
| | GKVHKINIELAREVGKNHSQRAKIEKEQNENYKAKKDAELECEKLGL | |
| | KINSKNILKLRLFKEQKEFCAYSGEKIKISDLQDEKMLEIDAIYPYS | |
| | RSFDDSYMNKVLVFTKQNQEKLNQTPFEAFGNDSAKWQKIEVLAKNL | |
| | PTKKQKRILDKNYKDKEQKNFKDRNLNDTRYIARLVLNYTKDYLDFL | |
| | PLSDDENTKLNDTQKGSKVHVEAKSGMLTSALRHTWGFSAKDRNNHL | |
| | HHAIDAVIIAYANNSIVKAFSDFKKEQESNSAELYAKKISELDYKNK | |
| | RKFFEPFSGFRQKVLDKIDEIFVSKPERKKPSGALHEETFRKEEEFY | |
| | QSYGGKEGVLKALELGKIRKVNGKIVKNGDMFRVDIFKHKKTNKFYA | |
| | VPIYTMDFALKVLPNKAVARSKKGEIKDWILMDENYEFCFSLYKDSL | |
| | ILIQTKDMQEPEFVYYNAFTSSTVSLIVSKHDNKFETLSKNQKILFK | |
| | NANEKEVIAKSIGIQNLKVFEKYIVSALGEVTKAEFRQREDFKK | |
| Corynebacterium | MKYHVGIAVGTFSVGLAAIEVDDAGMPIKTLSLVSHIHDSGLDPDEI | 6 |
| diphtheria | KSAVTRLASSGIARRTRRLYRRKRRRLQQLDKFIQRQGWPVIELEDY | |
| dCas9 | SDPLYPWKVRAELAASYIADEKERGEKLSVALRHIARHRGWRNPYAK | |
| (D8A, H573A) | VSSLYLPDGPSDAFKAIREEIKRASGQPVPETATVGQMVTLCELGTL | |
| | KLRGEGGVLSARLQQSDYAREIQEICRMQEIGQELYRKIIDVVFAAE | |
| | SPKGSASSRVGKDPLQPGKNRALKASDAFQRYRIAALIGNLRVRVDG | |
| | EKRILSVEEKNLVFDHLVNLTPKKEPEWVTIAEILGIDRGQLIGTAT | |
| | MTDDGERAGARPPTHDTNRSIVNSRIAPLVDWWKTASALEQHAMVKA | |
| | LSNAEVDDFDSPEGAKVQAFFADLDDDVHAKLDSLHLPVGRAAYSED | |
| | TLVRLTRRMLSDGVDLYTARLQEFGIEPSWTPPTPRIGEPVGNPAVD | |
| | RVLKTVSRWLESATKTWGAPERVIIEHVREGFVTEKRAREMDGDMRR | |
| | RAARNAKLFQEMQEKLNVQGKPSRADLWRYQSVQRQNCQCAYCGSPI | |
| | TFSNSEMDAIVPRAGQGSTNTRENLVAVCHRCNQSKGNTPFAIWAKN | |
| | TSIEGVSVKEAVERTRHWVTDTGMRSTDFKKFTKAVVERFQRATMDE | |
| | EIDARSMESVAWMANELRSRVAQHFASHGTTVRVYRGSLTAEARRAS | |
| | GISGKLKFFDGVGKSRLDRRHHAIDAAVIAFTSDYVAETLAVRSNLK | |
| | QSQAHRQEAPQWREFTGKDAEHRAAWRVWCQKMEKLSALLTEDLRDD | |
| | RVVVMSNVRLRLGNGSAHKETIGKLSKVKLSSQLSVSDIDKASSEAL | |
| | WCALTREPGFDPKEGLPANPERHIRVNGTHVYAGDNIGLFPVSAGSI | |
| | ALRGGYAELGSSFHHARVYKITSGKKPAFAMLRVYTIDLLPYRNQDL | |
| | FSVELKPQTMSMRQAEKKLRDALATGNAEYLGWLVVDDELVVDTSKI | |
| | ATDQVKAVEAELGTIRRWRVDGFFSPSKLRLRPLQMSKEGIKKESAP | |
| | ELSKIIDRPGWLPAVNKLFSDGNVTVVRRDSLGRVRLESTAHLPVTW | |
| | KVQ | |
| Eubacterium | MGYTVGLAIGVASVGVAVLDENDNIVEAVSNIFDEADTSNNKVRRTL | 7 |
| ventriosum | REGRRTKRRQKTRIEDFKQLWETSGYIIPHKLHLNIIELRNKGLTEL | |
| dCas9 | LSLDELYCVLLSMLKHRGISYLEDADDGEKGNAYKKGLAFNEKQLKE | |
| (D8A, H592A) | KMPCEIQLERMKKYGKYHGEFIIEINDEKEYQSNVFTTKAYKKELEK | |
| | IFETQRCNGNKINTKFIKKYMEIYERKREYYIGPGNEKSRTDYGIYT | |
| | TRTDEEGNFIDEKNIFGKLIGKCSVYPEEYRASSASYTAQEFNLLND | |
| | LNNLKINNEKLTEFQKKEIVEIIKDASSVNMRKIIKKVIDEDIEQYS | |
| | GARIDKKGKEIYHTFEIYRKLKKELKTINVDIDSFTREELDKTMDIL | |
| | TLNTERESIVKAFDEQKFVYEENLIKKLIEFRKNNQRLFSGWHSFSY | |
| | KAMLQLIPVMYKEPKEQMQLLTEMNVFKSKKEKYVNYKYIPENEVVK | |
| | EIYNPVVVKSIRTTVKILNALIKKYGYPESVVIEMPRDKNSDDEKEK | |
| | IDMNQKKNQEEYEKILNKIYDEKGIEITNKDYKKQKKLVLKLKLWNE | |
| | QEGLCLYSGKKIAIEDLLNHPEFFEIDAIIPKSISLDDSRSNKVLVY | |
| | KTENSIKENDTPYHYLTRINGKWGFDEYKANVLELRRRGKIDDKKVN | |
| | NLLCMEDITKIDVVKGFINRNLNDTRYASRVVLNEMQSFFESRKYCN | |
| | TKVKVIRGSLTYQMRQDLHLKKNREESYSHHAVDAMLIAFSQKGYEA | |
| | YRKIQKDCYDFETGEILDKEKWNKYIDDDEFDDILYKERMNEIRKKI | |
| | IEAEEKVKYNYKIDKKCNRGLCNQTIYGTREKDGKIHKISSYNIYDD | |
| | KECNSLKKMINSGKGSDLLMYNNDPKTYRDMLKILETYSSEKNPFVA | |
| | YNKETGDYFRKYSKNHNGPKVEKVKYYSGQINSCIDISHKYGHAKNS | |
| | KKVVLVSLNPYRTDVYYDNDTGKYYLVGVKYNHIKCVGNKYVIDSET | |
| | YNELLRKEGVLNSDENLEDLNSKNITYKFSLYKNDIIQYEKGGEYYT | |
| | ERFLSRIKEQKNLIETKPINKPNFQRKNKKGEWENTRNQIALAKTKY | |
| | VGKLVTDVLGNCYIVNMEKFSLVVDK | |
| Streptococcus | MTNGKILGLAIGIASVGVGIIEAKTGKVVHANSRLFSAANAENNAER | 8 |
| pasteurianus | RGFRGSRRLNRRKKHRVKRVRDLFEKYGIVTDFRNLNLNPYELRVKG | |
| dCas9 | LTEQLKNEELFAALRTISKRRGISYLDDAEDDSTGSTDYAKSIDENR | |
| (D10A, H599A) | RLLKNKTPGQIQLERLEKYGQLRGNFTVYDENGEAHRLINVFSTSDY | |
| | EKEARKILETQADYNKKITAEFIDDYVEILTQKRKYYHGPGNEKSRT | |
| | DYGRFRTDGTTLENIFGILIGKCNFYPDEYRASKASYTAQEYNFLND | |
| | LNNLKVSTETGKLSTEQKESLVEFAKNTATLGPAKLLKEIAKILDCK | |
| | VDEIKGYREDDKGKPDLHTFEPYRKLKFNLESINIDDLSREVIDKLA | |
| | DILTLNTEREGIEDAIKRNLPNQFTEEQISEIIKVRKSQSTAFNKGW | |
| | HSFSAKLMNELIPELYATSDEQMTILTRLEKFKVNKKSSKNTKTIDE | |
| | KEVTDEIYNPVVAKSVRQTIKIINAAVKKYGDFDKIVIEMPRDKNAD | |
| | DEKKFIDKRNKENKKEKDDALKRAAYLYNSSDKLPDEVFHGAKQLET | |
| | KIRLWYQQGERCLYSGKPISIQELVHNSNNFEIDHILPLSLSFDDSL | |
| | ANKVLVYAWTNQEKGQKTPYQVIDSMDAAWSFREMKDYVLKQKGLGK | |
| | KKRDYLLTTENIDKIEVKKKFIERNLVDTRYASRVVLNSLQSALREL | |
| | GKDTKVSVVRGQFTSQLRRKWKIDKSRETYHHHAVDALIIAASSQLK | |
| | LWEKQDNPMFVDYGKNQVVDKQTGEILSVSDDEYKELVFQPPYQGFV | |
| | NTISSKGFEDEILFSYQVDSKYNRKVSDATIYSTRKAKIGKDKKEET | |
| | YVLGKIKDIYSQNGFDTFIKKYNKDKTQFLMYQKDSLTWENVIEVIL | |
| | RDYPTTKKSEDGKNDVKCNPFEEYRRENGLICKYSKKGKGTPIKSLK | |
| | YYDKKLGNCIDITPEESRNKVILQSINPWRADVYFNPETLKYELMGL | |
| | KYSDLSFEKGTGNYHISQEKYDAIKEKEGIGKKSEFKFTLYRNDLIL | |
| | IKDIASGEQEIYRFLSRTMPNVNHYVELKPYDKEKFDNVQELVEALG | |
| | EADKVGRCIKGLNKPNISIYKVRTDVLGNKYFVKKKGDKPKLDFKNN | |
| | KK | |
| Lactobacillus | MTDRISLGLAIGVASVGFSVLDIDKGKVIELGARLFNATVAAGNQDR | 9 |
| farciminis | RDMRGSRRLLNRNKQRRKDVSKLFQEYGLLDNFDRDNFHTAFNNNWN | |
| dCas9 | PYELRVKGLTKQLNKEELADSLYQIIKRRGISYALKDADANEDGTDY | |
| (D10A, H611A) | SSSLKINSQELIEKTPAQIQLQRLNDYGKVRGKVIVGDDIDDQKVLL | |
| | NVFPTNAYEKEARQIIATQQQFYPEILTGEFVKEYCQILTRKRDYFV | |
| | GPGNEKSRTDYGIYKTDGRTLDNLFEELIGHDKIYPDELRASAASYT | |
| | AQLFNVLNDLNNLRILSYEDGKLTQIDKEKIIAEIKNNTTVVNMLNV | |
| | IKKVTGCQKDDIKGFRLNEKDKPEISSMPIYRKAHKDFLKVGIDITD | |
| | WPIDFIDRLSFILTLNTENGEIRKQINKRLVPDFDELNDDLVQLIID | |
| | NKDSFDIKTNNKWHRFSLKTMNKLIPTMIERPVEQMTLLTEMGLIKK | |
| | DTKRFEGNKYLPYKEIANDIFNPVASKSVREALKIVNAVLKKYGHIE | |
| | YIVIEMPRDKNLDEERKQIADFQKKNKKIKDAAFNAFVKAVGSKKNV | |
| | KIALSKNRKLQMGIRLWHQQQGIDPYNGEEINANDLISNPDKFEIDA | |
| | IIPQSISFDDGINNKTLCFASMNQVKGQKTPYEFLNEGHGQGYAKFK | |
| | AIVSKNKNFSKAKRDNYLFEENVSNIETRKRFLSRNLVDTRYSSRVV | |
| | LNSLQEFFHEKSDDTKVTVIRGKFTSNMRKHWHIDKTRDTFHHHAID | |
| | ASIIAATPFLRMWKKGSTIFPTKINETSIDIETGEILDDKNFDKVMY | |
| | EEPYSGFISEIMNADDRIKFSHQVDKKMNRKVSDATIYSTRIGKLSK | |
| | DKKDAEYVVAKVKDIYSVDGYENFKKVYKKDKTKELLYKYDPRTFSE | |
| | LEKIVIDYPDKIEKVQTNGKIKAVDISPFELYRRDNGMVRKYSKKTG | |
| | PAIKQLKYLDKKLGSHIDITPKNANNKHVVLQSLKPWRTDVYLNHET | |
| | GEYEIMGIKYSDLKFNKNEGYGIKKDKYLKIKNIEGVSENSEFMFSL | |
| | YRKDRIKVQDMETNESVELLFWSRNFSNKKYAELKPISQIDNDKVLP | |
| | IYGRGRLIKRLIPKNCKIWKVNTSILGDTYYLEKESNYPQKILD | |
| Sphaerochaeta | MSKKVSRRYEEQAQEICQRLGSRPYSIGLALGVGSIGVAVAAYDPIK | 10 |
| globosa | KQPSDLVFVSSRIFIPSTGAAERRQKRGQRNSLRHRANRLKFLWKLL | |
| dCas9 | AERNLMLSYSEQDVPDPARLRFEDAVVRANPYELRLKGLNEQLTLSE | |
| (D30A, H635A) | LGYALYHIANHRGSSSVRTFLDEEKSSDDKKLEEQQAMTEQLAKEKG | |
| | ISTFIEVLTAFNTNGLIGYRNSESVKSKGVPVPTRDIISNEIDVLLQ | |
| | TQKQFYQEILSDEYCDRIVSAILFENEKIVPEAGCCPYFPDEKKLPR | |
| | CHFLNEERRLWEAINNARIKMPMQEGAAKRYQSASFSDEQRHILFHI | |
| | ARSGTDITPKLVQKEFPALKTSIIVLQGKEKAIQKIAGFRFRRLEEK | |
| | SFWKRLSEEQKDDFFSAWTNTPDDKRLSKYLMKHLLLTENEVVDALK | |
| | TVSLIGDYGPIGKTATQLLMKHLEDGLTYTEALERGMETGEFQELSV | |
| | WEQQSLLPYYGQILTGSTQALMGKYWHSAFKEKRDSEGFFKPNTNSD | |
| | EEKYGRIANPVVHQTLNELRKLMNELITILGAKPQEITVELARELKV | |
| | GAEKREDIIKQQTKQEKEAVLAYSKYCEPNNLDKRYIERFRLLEDQA | |
| | FVCPYCLEHISVADIAAGRADVDAIFPRDDTADNSYGNKVVAHRQCN | |
| | DIKGKRTPYAAFSNTSAWGPIMHYLDETPGMWRKRRKFETNEEEYAK | |
| | YLQSKGFVSRFESDNSYIAKAAKEYLRCLFNPNNVTAVGSLKGMETS | |
| | ILRKAWNLQGIDDLLGSRHWSKDADTSPTMRKNRDDNRHHGLDAIVA | |
| | LYCSRSLVQMINTMSEQGKRAVEIEAMIPIPGYASEPNLSFEAQREL | |
| | FRKKILEFMDLHAFVSMKTDNDANGALLKDTVYSILGADTQGEDLVF | |
| | VVKKKIKDIGVKIGDYEEVASAIRGRITDKQPKWYPMEMKDKIEQLQ | |
| | SKNEAALQKYKESLVQAAAVLEESNRKLIESGKKPIQLSEKTISKKA | |
| | LELVGGYYYLISNNKRTKTFVVKEPSNEVKGFAFDTGSNLCLDFYHD | |
| | AQGKLCGEIIRKIQAMNPSYKPAYMKQGYSLYVRLYQGDVCELRASD | |
| | LTEAESNLAKTTHVRLPNAKPGRIFVIIITFTEMGSGYQIYFSNLAK | |
| | SKKGQDTSFTLTTIKNYDVRKVQLSSAGLVRYVSPLLVDKIEKDEVA | |
| | LCGE | |
| Azospirillum | MARPAFRAPRREHVNGWTPDPHRISKPFFILVSWHLLSRVVIDSSSG | 11 |
| (strain B510) | CFPGTSRDHTDKFAEWECAVQPYRLSFALGINSIGWGLLNLDRQGKP | |
| dCas9 | REIRALGSRIFSDGRDPQDKASLAVARRLARQMRRRRDRYLTRRTRL | |
| (D75A, H680A) | MGALVRFGLMPADPAARKRLEVAVDPYLARERATRERLEPFEIGRAL | |
| | FHLNQRRGYKPVRTATKPDEEAGKVKEAVERLEAAIAAAGAPTLGAW | |
| | FAWRKTRGETLRARLAGKGKEAAYPFYPARRMLEAEFDTLWAEQARH | |
| | HPDLLTAEAREILRHRIFHQRPLKPPPVGRCTLYPDDGRAPRALPSA | |
| | QRLRLFQELASLRVIHLDLSERPLTPAERDRIVAFVQGRPPKAGRKP | |
| | GKVQKSVPFEKLRGLLELPPGTGFSLESDKRPELLGDETGARIAPAF | |
| | GPGWTALPLEEQDALVELLLTEAEPERAIAALTARWALDEATAAKLA | |
| | GATLPDFHGRYGRRAVAELLPVLERETRGDPDGRVRPIRLDEAVKLL | |
| | RGGKDHSDFSREGALLDALPYYGAVLERHVAFGTGNPADPEEKRVGR | |
| | VANPTVHIALNQLRHLVNAILARHGRPEEIVIELARDLKRSAEDRRR | |
| | EDKRQADNQKRNEERKRLILSLGERPTPRNLLKLRLWEEQGPVENRR | |
| | CPYSGETISMRMLLSEQVDIDAILPFSVSLDDSAANKVVCLREANRI | |
| | KRNRSPWEAFGHDSERWAGILARAEALPKNKRWRFAPDALEKLEGEG | |
| | GLRARHLNDTRHLSRLAVEYLRCVCPKVRVSPGRLTALLRRRWGIDA | |
| | ILAEADGPPPEVPAETLDPSPAEKNRADHRHHALDAVVIGCIDRSMV | |
| | QRVQLAAASAEREAAAREDNIRRVLEGFKEEPWDGFRAELERRARTI | |
| | VVSHRPEHGIGGALHKETAYGPVDPPEEGFNLVVRKPIDGLSKDEIN | |
| | SVRDPRLRRALIDRLAIRRRDANDPATALAKAAEDLAAQPASRGIRR | |
| | VRVLKKESNPIRVEHGGNPSGPRSGGPFHKLLLAGEVHHVDVALRAD | |
| | GRRWVGHWVTLFEAHGGRGADGAAAPPRLGDGERFLMRLHKGDCLKL | |
| | EHKGRVRVMQVVKLEPSSNSVVVVEPHQVKTDRSKHVKISCDQLRAR | |
| | GARRVTVDPLGRVRVHAPGARVGIGGDAGRTAMEPAEDIS | |
| Gluconacetobacter | MIDESLTFGIALGIGSCGWAVLRRPSAFGRKGVIEGMGSWCFDVPET | 12 |
| diazotrophicus | SKERTPTNQIRRSNRLLRRVIRRRRNRMAAIRRLLHAAGLLPSTDSD | |
| dCas9 | ALKRPGHDPWELRARGLDKPLKPVEFAVVLGHIAKRRGFKSAAKRKA | |
| (D11A, H587A) | TNISSDDKKMLTALEATRERLGRYRTVGEMFARDPDFASRRRNREGK | |
| | YDRTTARDDLEHEVHALFAAQRRLGQGFASPELEEAFTASAFHQRPM | |
| | QDSERLVGFCPFERTEKRAAKLTPSFERFRLLARLLNLRITTPDGER | |
| | PLTVDEIALVTRDLGKTAKLSIKRVRTLIGLEDNQRFTTIRPEDEDR | |
| | DIVARTGGAMTGTATLRKALGEALWTDMQERPEQLDAIVQVLSFFEA | |
| | NETITEKLREIGLTLAVLDVLLTALDAGVFAKFKGAAHISTKAARNL | |
| | LPHLEQGRRYDEACTMAGYDHAASRLSHHGQIVAKTQFNALVTEIGE | |
| | SIANPIARKALIEGLKQIWAMRNHWGLPGSIHVELARDVGNSIEKRR | |
| | EIEKHIEKNTALRARERREVHDLLDLEDVNGDTLLRYRLWKEQGGKC | |
| | LYTGKAIHIRQIAATDNSVQVDAILPWSRFGDDSFNNKTLCLASANQ | |
| | QKKRSTPYEWLSGQTGDAWNAFVQRIETNKELRGFKKRNYLLKNAKE | |
| | AEEKFRSRNLNDTRYAARLFAEAVKLLYAFGERQEKGGNRRVFTRPG | |
| | ALTAALRQAWGVESLKKQDGKRINDDRHHALDALTVAAVDEAEIQRL | |
| | TKSFHEWEQQGLGRPLRRVEPPWESFRADVEATYPEVFVARPERRRA | |
| | RGEGHAATIRQVKERECTPIVFERKAVSSLKEADLERIKDGERNEAI | |
| | VEAIRSWIATGRPADAPPRSPRGDIITKIRLATTIKAAVPVRGGTAG | |
| | RGEMVRADVFSKPNRRGKDEWYLVPVYPHQIMNRKAWPKPPMRSIVA | |
| | NKDEDEWTEVGPEHQFRFSLYPRSNIEIIRPSGEVIEGYFVGLHRNT | |
| | GALIPTPVGPDSYVIA | |
| Neisseria | MAAFKPNPMNYILGLAIGIASVGWAIVEIDEEENPIRLIDLGVRVFE | 13 |
| cinerea | RAEVPKTGDSLAAARRLARSVRRLTRRRAHRLLRARRLLKREGVLQA | |
| dCas9 | ADFDENGLIKSLPNTPWQLRAAALDRKLTPLEWSAVLLHLIKHRGYL | |
| (D16A, H588A) | SQRKNEGETADKELGALLKGVADNTHALQTGDFRTPAELALNKFEKE | |
| | SGHIRNQRGDYSHTFNRKDLQAELNLLFEKQKEFGNPHVSDGLKEGI | |
| | ETLLMTQRPALSGDAVQKMLGHCTFEPTEPKAAKNTYTAERFVWLTK | |
| | LNNLRILEQGSERPLTDTERATLMDEPYRKSKLTYAQARKLLDLDDT | |
| | AFFKGLRYGKDNAEASTLMEMKAYHAISRALEKEGLKDKKSPLNLSP | |
| | ELQDEIGTAFSLFKTDEDITGRLKDRVQPEILEALLKHISFDKFVQI | |
| | SLKALRRIVPLMEQGNRYDEACTEIYGDHYGKKNTEEKIYLPPIPAD | |
| | EIRNPVVLRALSQARKVINGVVRRYGSPARIHIETAREVGKSFKDRK | |
| | EIEKRQEENRKDREKSAAKFREYFPNFVGEPKSKDILKLRLYEQQHG | |
| | KCLYSGKEINLGRLNEKGYVEIDAALPFSRTWDDSFNNKVLALGSEN | |
| | QNKGNQTPYEYFNGKDNSREWQEFKARVETSRFPRSKKQRILLQKFD | |
| | EDGFKERNLNDTRYINRFLCQFVADHMLLTGKGKRRVFASNGQITNL | |
| | LRGFWGLRKVRAENDRHHALDAVVVACSTIAMQQKITRFVRYKEMNA | |
| | FDGKTIDKETGEVLHQKAHFPQPWEFFAQEVMIRVFGKPDGKPEFEE | |
| | ADTPEKLRTLLAEKLSSRPEAVHKYVTPLFISRAPNRKMSGQGHMET | |
| | VKSAKRLDEGISVLRVPLTQLKLKDLEKMVNREREPKLYEALKARLE | |
| | AHKDDPAKAFAEPFYKYDKAGNRTQQVKAVRVEQVQKTGVWVHNHNG | |
| | IADNATIVRVDVFEKGGKYYLVPIYSWQVAKGILPDRAVVQGKDEED | |
| | WTVMDDSFEFKFVLYANDLIKLTAKKNEFLGYFVSLNRATGAIDIRT | |
| | HDTDSTKGKNGIFQSVGVKTALSFQKYQIDELGKEIRPCRLKKRPPV | |
| | R | |
| Roseburia | MRENGSDERRRNMDEKMDYRIGLAIGIASVGWAVLQNNSDDEPVRIV | 14 |
| intestinalis | DLGVRIFDTAEIPKTGESLAGPRRAARTTRRRLRRRKHRLDRIKWLF | |
| dCas9 | ENQGLINIDDFLKRYNMAGLPDVYQLRYEALDRKLTDEELAQVLLHI | |
| (D24A, H628A) | AKHRGFRSTRKAETAAKENGAVLKATDENQKRMQEKGYRTVGEMIYL | |
| | DEAFRTGCSWSEKGYILTPRNKAENYQHTMLRAMLVEEVKEIFSSQR | |
| | RLGNEKATEELEEKYLEIMTSQRSFDLGPGMQPDGKPSPYAMEGFSD | |
| | RVGKCTFLGDQGELRGAKGTYTAEYFVALQKINHTKLVNQDGETRNF | |
| | TEEERRALTLLLFTQKEVKYAAVRKKLGLPEDILFYNLNYKKAATKE | |
| | EQQKENQNTEKAKFIGMPYYHDYKKCLEERVKYLTENEVRDLFDEIG | |
| | MILTCYKNDDSRTERLAKLGLVPIEMEGLLAYTPTKFQHLSMKAMRN | |
| | IIPFLEKGMTYDKACEEAGYDFKADSKGTKQKLLTGENVNQTINEIT | |
| | NPVVKRSVSQTVKVINAIIRTYGSPQAINIELAREMSKTFEERRKIK | |
| | GDMEKRQKNNEDVKKQIQELGKLSPTGQDILKYRLWQEQQGICMYSG | |
| | KTIPLEELFKPGYDIDAILPYSITFDDSFRNKVLVTSQENRQKGNRT | |
| | PYEYMGNDEQRWNEFETRVKTTIRDYKKQQKLLKKHFSEEERSEFKE | |
| | RNLTDTKYITTVIYNMIRQNLEMAPLNRPEKKKQVRAVNGAITAYLR | |
| | KRWGLPQKNRETDTHHAMDAVVIACCTDGMIQKISRYTKVRERCYSK | |
| | GTEFVDAETGEIFRPEDYSRAEWDEIFGVHIPKPWETFRAELDVRMG | |
| | DDPKGFLDTHSDVALELDYPEYIYENLRPIFVSRMPNHKVTGAAHAD | |
| | TIRSPRHFKDEGIVLTKTALTDLKLDKDGEIDGYYNPQSDLLLYEAL | |
| | KKQLLLYGNDAKKAFAQDFHKPKADGTEGPVVRKVKIQKKQTMGVFV | |
| | DSGNGIAENGGMVRIDVFRVNGKYYFVPVYTADVVKKVLPNRASTAH | |
| | KPYGEWKVMEDKDFLFSLYSRDLIHIKSKKDIPIKMVNGGMEGIKET | |
| | YAYYIGADISAANIQGIAHDSRYKFRGLGIQSLDVLEKCQIDVLGHV | |
| | SVVRSEKRMGFS | |
| Parvibaculum | MERIFGFAIGTTSIGFSVIDYSSTQSAGNIQRLGVRIFPEARDPDGT | 15 |
| lavamentivorans | PLNQQRRQKRMMRRQLRRRRIRRKALNETLHEAGFLPAYGSADWPVV | |
| dCas9 | MADEPYELRRRGLEEGLSAYEFGRAIYHLAQHRHFKGRELEESDTPD | |
| (D8A, H601A) | PDVDDEKEAANERAATLKALKNEQTTLGAWLARRPPSDRKRGIHAHR | |
| | NVVAEEFERLWEVQSKFHPALKSEEMRARISDTIFAQRPVFWRKNTL | |
| | GECRFMPGEPLCPKGSWLSQQRRMLEKLNNLAIAGGNARPLDAEERD | |
| | AILSKLQQQASMSWPGVRSALKALYKQRGEPGAEKSLKFNLELGGES | |
| | KLLGNALEAKLADMFGPDWPAHPRKQEIRHAVHERLWAADYGETPDK | |
| | KRVIILSEKDRKAHREAAANSFVADFGITGEQAAQLQALKLPTGWEP | |
| | YSIPALNLFLAELEKGERFGALVNGPDWEGWRRTNFPHRNQPTGEIL | |
| | DKLPSPASKEERERISQLRNPTVVRTQNELRKVVNNLIGLYGKPDRI | |
| | RIEVGRDVGKSKREREEIQSGIRRNEKQRKKATEDLIKNGIANPSRD | |
| | DVEKWILWKEGQERCPYTGDQIGFNALFREGRYEVEAIWPRSRSFDN | |
| | SPRNKTLCRKDVNIEKGNRMPFEAFGHDEDRWSAIQIRLQGMVSAKG | |
| | GTGMSPGKVKRFLAKTMPEDFAARQLNDTRYAAKQILAQLKRLWPDM | |
| | GPEAPVKVEAVTGQVTAQLRKLWTLNNILADDGEKTRADHRHHAIDA | |
| | LTVACTHPGMTNKLSRYWQLRDDPRAEKPALTPPWDTIRADAEKAVS | |
| | EIVVSHRVRKKVSGPLHKETTYGDTGTDIKTKSGTYRQFVTRKKIES | |
| | LSKGELDEIRDPRIKEIVAAHVAGRGGDPKKAFPPYPCVSPGGPEIR | |
| | KVRLTSKQQLNLMAQTGNGYADLGSNHHIAIYRLPDGKADFEIVSLF | |
| | DASRRLAQRNPIVQRTRADGASFVMSLAAGEAIMIPEGSKKGIWIVQ | |
| | GVWASGQVVLERDTDADHSTTTRPMPNPILKDDAKKVSIDPIGRVRP | |
| | SND | |
| Nitratifractor | MKKILGVALGITSFGYAILQETGKDLYRCLDNSVVMRNNPYDEKSGE | 16 |
| salsuginis | SSQSIRSTQKSMRRLIEKRKKRIRCVAQTMERYGILDYSETMKINDP | |
| (strain DSM | KNNPIKNRWQLRAVDAWKRPLSPQELFAIFAHMAKHRGYKSIATEDL | |
| 16511) dCas9 | IYELELELGLNDPEKESEKKADERRQVYNALRHLEELRKKYGGETIA | |
| (D8A, H611A) | QTIHRAVEAGDLRSYRNHDDYEKMIRREDIEEEIEKVLLRQAELGAL | |
| | GLPEEQVSELIDELKACITDQEMPTIDESLFGKCTFYKDELAAPAYS | |
| | YLYDLYRLYKKLADLNIDGYEVTQEDREKVIEWVEKKIAQGKNLKKI | |
| | THKDLRKILGLAPEQKIFGVEDERIVKGKKEPRTFVPFFFLADIAKF | |
| | KELFASIQKHPDALQIFRELAEILQRSKTPQEALDRLRALMAGKGID | |
| | TDDRELLELFKNKRSGTRELSHRYILEALPLFLEGYDEKEVQRILGF | |
| | DDREDYSRYPKSLRHLHLREGNLFEKEENPINNHAVKSLASWALGLI | |
| | ADLSWRYGPFDEIILETTRDALPEKIRKEIDKAMREREKALDKIIGK | |
| | YKKEFPSIDKRLARKIQLWERQKGLDLYSGKVINLSQLLDGSADIEA | |
| | IVPQSLGGLSTDYNTIVTLKSVNAAKGNRLPGDWLAGNPDYRERIGM | |
| | LSEKGLIDWKKRKNLLAQSLDEIYTENTHSKGIRATSYLEALVAQVL | |
| | KRYYPFPDPELRKNGIGVRMIPGKVTSKTRSLLGIKSKSRETNFHHA | |
| | EDALILSTLTRGWQNRLHRMLRDNYGKSEAELKELWKKYMPHIEGLT | |
| | LADYIDEAFRRFMSKGEESLFYRDMEDTIRSISYWVDKKPLSASSHK | |
| | ETVYSSRHEVPTLRKNILEAFDSLNVIKDRHKLTTEEFMKRYDKEIR | |
| | QKLWLHRIGNTNDESYRAVEERATQIAQILTRYQLMDAQNDKEIDEK | |
| | FQQALKELITSPIEVTGKLLRKMRFVYDKLNAMQIDRGLVETDKNML | |
| | GIHISKGPNEKLIFRRMDVNNAHELQKERSGILCYLNEMLFIFNKKG | |
| | LIHYGCLRSYLEKGQGSKYIALFNPRFPANPKAQPSKFTSDSKIKQV | |
| | GIGSATGIIKAHLDLDGHVRSYEVFGTLPEGSIEWFKEESGYGRVED | |
| | DPHH | |
| Campylobacter | MKILGFAIGINSIGWAFVENDELKDCGVRIFTEAENPSNKESLALPR | 17 |
| lari dCas9 | RNARSSRRRLKRRKARLVAIKRILAKELKLNFKDYVAADGELPKAYE | |
| (D7A, H567A) | GKLTSIYELRYKALIQQLETKDLARVILHIAKHRGYMNKNEKKSNDT | |
| | KKGKILSALKNNALKLENYQSVGEYFYKEFFQKYKENTKNFIKIRNT | |
| | KDNYNNCVLSSDLEKELKLILEKQKEFGYNYSEDFINEILKVAFFQR | |
| | PLKDFSHLVGACTFFEEEKRACKNSYSAWEFVALTKIINEIKSLEKI | |
| | SGEIVPTQTINEILNLILDKGSITYKKFRSCINLHESISFKSLKYDK | |
| | ENAENAKLIDFRKLVEFKKALGVHSLSRQELDQISTHITLIKDNVKL | |
| | KTVLEKYNLSNEQINNLLEIEFNDYINLSFKALGMILPLMREGKRYY | |
| | EACEITNLKPKTVDEKEDFLPAFCDSIFAHELSNPVVNRAISEYRKV | |
| | LNALLKKYGKVHKIHLELARDVGLSKKAREKIEDQQKNNKAINDWAL | |
| | KECENIGIKASAKNILKLKLWKEQKEICIYSGNKISIEHLKDEKALE | |
| | VDAIYPYSRSFDDSFINKVLVFTKENQEKLNKTPFEAFGKNIEKWSK | |
| | IQTLAQNLPYKKKNKILDENFKDKQQQDFISRNLNDTRYITTLIAKY | |
| | TKEYLNFLPLSENENTNLKSGEKGSKIHIQTISGMLTSVLRHTWGFD | |
| | KKDRNNHLHHALDAIIVAYSTSSIIKAFSDFKKNQELLKARFYAKEL | |
| | TSDNYKHQVKFFEPFKGFREKILSKIDEIFVSTPPRKRARGALHEET | |
| | FYSEDKMIKKYNSKEGLQIALSCGRVRKIGTKYVENDTMVRVDIFKK | |
| | QNKFYAIPIYVMDFALGILPNKIVITGKDKNNNPKQWQTIDESYEFC | |
| | FSLYKNDLILLQKKNMQEPEFAYYNSFDIDRSRIKIKKHDNKFENLT | |
| | SNQKLLFTNKDKGKNRTGIQNLKIFEKYIVTPLGDKIKADFQPRENI | |
| | SLKTSKKHGL | |
| Streptococcus | MTKPYSIGLAIGTNSVGWAVTTDNYKVPSKKMKVLGNTSKKYIKKNL | 18 |
| thermophilus | LGVLLFDSGITAEGRRLKRTARRRYTRRRNRILYLQEIFSTEMATLD | |
| (strain LMD-9) | DAFFQRLDDSFLVPDDKRDSKYPIFGNLVEEKAYHDEFPTIYHLRKY | |
| dCas9 | LADSTKKADLRLVYLALAHMIKYRGHFLIEGEFNSKNNDIQKNFQDF | |
| (D10A, H847A) | LDTYNAIFESDLSLENSKQLEEIVKDKISKLEKKDRILKLFPGEKNS | |
| | GIFSEFLKLIVGNQADERKCFNLDEKASLHFSKESYDEDLETLLGYI | |
| | GDDYSDVFLKAKKLYDAILLSGELTVTDNETEAPLSSAMIKRYNEHK | |
| | EDLALLKEYIRNISLKTYNEVFKDDTKNGYAGYIDGKTNQEDFYVYL | |
| | KKLLAEFEGADYFLEKIDREDFLRKQRTFDNGSIPYQIHLQEMRAIL | |
| | DKQAKFYPFLAKNKERIEKILTFRIPYYVGPLARGNSDFAWSIRKRN | |
| | EKITPWNFEDVIDKESSAEAFINRMTSFDLYLPEEKVLPKHSLLYET | |
| | FNVYNELTKVRFIAESMRDYQFLDSKQKKDIVRLYFKDKRKVTDKDI | |
| | IEYLHAIYGYDGIELKGIEKQFNSSLSTYHDLLNIINDKEFLDDSSN | |
| | EAIIEEIIHTLTIFEDREMIKQRLSKFENIFDKSVLKKLSRRHYTGW | |
| | GKLSAKLINGIRDEKSGNTILDYLIDDGISNRNFMQLIHDDALSFKK | |
| | KIQKAQIIGDEDKGNIKEVVKSLPGSPAIKKGILQSIKIVDELVKVM | |
| | GGRKPESIVVEMARENQYTNQGKSNSQQRLKRLEKSLKELGSKILKE | |
| | NIPAKLSKIDNNALQNDRLYLYYLQNGKDMYTGDDLDIDRLSNYDID | |
| | AIIPQAFLKDNSIDNKVLVSSASNRGKSDDVPSLEVVKKRKTFWYQL | |
| | LKSKLISQRKFDNLTKAERGGLSPEDKAGFIQRQLVETRQITKHVAR | |
| | LLDEKFNNKKDENNRAVRTVKIITLKSTLVSQFRKDFELYKVREIND | |
| | FHHAHDAYLNAVVASALLKKYPKLEPEFVYGDYPKYNSFRERKSATE | |
| | KVYFYSNIMNIFKKSISLADGRVIERPLIEVNEETGESVWNKESDLA | |
| | TVRRVLSYPQVNVVKKVEEQNHGLDRGKPKGLFNANLSSKPKPNSNE | |
| | NLVGAKEYLDPKKYGGYAGISNSFTVLVKGTIEKGAKKKITNVLEFQ | |
| | GISILDRINYRKDKLNFLLEKGYKDIELIIELPKYSLFELSDGSRRM | |
| | LASILSTNNKRGEIHKGNQIFLSQKFVKLLYHAKRISNTINENHRKY | |
| | VENHKKEFEELFYYILEFNENYVGAKKNGKLLNSAFQSWQNHSIDEL | |
| | CSSFIGPTGSERKGLFELTSRGSAADFEFLGVKIPRYRDYTPSSLLK | |
| | DATLIHQSVTGLYETRIDLAKLGEG | |

### Cas9 Fusion Proteins

The CRISPR/Cas9-based system may include a fusion molecule (e.g., DNMT3A-DNMT3L (3A3L)-dCas9-KRAB). The fusion molecule may comprise at least one DNA binding protein (e.g., dCas9), and at least one modulator of gene expression (e.g., KRAB, DNMT3A, DNMT3L, DNMT3A-DNMT3L fusion peptide). In some embodiments, the modulator of gene expression is chosen from a repressor of gene expression (e.g. KRAB), an activator of gene expression, or a modulator of epigenetic modification (e.g. DNMT3A, DNMT3L, DNMT3A-DNMT3L fusion peptide) or any combination thereof. Different modulators of gene expression are known in the art, see, e.g., Thakore et al., Nat Methods. 2016; 13:127-37, incorporated by reference herein in its entirety.

### Repressors of Gene Expression

In some embodiments, the modulator of gene expression comprises a repressor of gene expression. The repressor may be any known repressor of gene expression, for example, a repressor chosen from Kruppel associated box (KRAB) domain, mSin3 interaction domain (SID), MAX-interacting protein 1 (MXI1), a chromo shadow domain, an EAR-repression domain (SRDX), eukaryotic release factor 1 (ERF1), eukaryotic release factor 3 (ERF3), tetracycline repressor, the lad repressor, Catharanthus roseus G-box binding factors 1 and 2, Drosophila Groucho, Tripartite motif-containing 28 (TRTM28), Nuclear receptor co-repressor 1, Nuclear receptor co-repressor 2, or fragment or fusion thereof.

### Kruppel Associated Box (KRAB)

The KRAB domain is a type of transcriptional repression domains present in the N-terminal part of many zinc finger protein-based transcription factors. The KRAB domain functions as a transcriptional repressor when tethered to a target DNA by a DNA-binding domain. The KRAB domain is enriched in charged amino acids and can be divided into sub-domains A and B. The KRAB A and B sub-domains can be separated by variable spacer segments and many KRAB proteins contain only the A sub-domain. A sequence of 45 amino acids in the KRAB A sub-domain has been shown to be important for transcriptional repression. The B sub-domain does not repress transcription by itself but does potentiate the repression exerted by the KRAB A sub-domain. The KRAB domain recruits corepressors KAP1 (KRAB-associated protein-1, also known as transcription intermediary factor 1 beta, KRAB-A interacting protein and tripartite motif protein 28) and heterochromatin protein 1 (HP1), as well as other chromatin modulating proteins, leading to transcriptional repression through heterochromatin formation. In one embodiment, the methods and compositions disclosed herein include a fusion molecule comprising a dCas9 molecule fused to a KRAB domain or fragment thereof. In one embodiment, the KRAB domain or fragment thereof is fused to the N-terminus of the dCas9 molecule. In one embodiment, the KRAB domain or fragment thereof is fused to the C-terminus of the dCas9 molecule. In one embodiment, the KRAB domain or fragment thereof is fused to both the N-terminus and the C-terminus of the dCas9 molecule. In one embodiment, the fusion molecule comprises a KRAB domain comprising the sequence of SEQ ID NO: 22, a sequence substantially identical (e.g., at least 80%, 85%, 90%, 92%, 95%, 97%, 98%, 99% or higher identical) to SEQ ID NO: 22, or a sequence having one, two, three, four, five or more changes, e.g., amino acid substitutions, insertions, or deletions, relative to SEQ ID NO: 22, or any fragment thereof.

Exemplary KRAB domain sequence:

(SEQ ID NO: 22)
RTLVTFKDVFVDFTREEWKLLDTAQQIVYRNVMLENYKNLVSLGYQ
LTKPDVILRLEKGEEP

mSin3 Interaction Domain (SID)

The mSin3 interaction domain (SID) is an interaction domain that is present on several transcription repressor proteins. It interacts with the paired amphipathic alpha-helix 2 (PAH2) domain of mSin3, a transcriptional repressor domain that is attached to transcription repressor proteins such as the mSin3 A corepressor. In one embodiment, the methods and compositions disclosed herein include a fusion molecule comprising a dCas9 molecule fused to an mSin3 interaction domain or fragment thereof. In one embodiment, the methods and compositions disclosed herein include a fusion molecule comprising a dCas9 molecule fused to four concatenated mSin3 interaction domains (SID4X). In one embodiment, the four concatenated mSin3 interaction domains (SID4X) are fused to the C-terminus of the dCas9 molecule.

### MAX-Interacting Protein 1 (MX11)

Mxi1 is a repressor of MYC. Mxi1 antagonizes MYC transcriptional activity possibly by competing for binding to MYC-associated factor X (MAX), which binds to MYC and is required for MYC to function. In one embodiment, the methods and compositions disclosed herein include a fusion molecule comprising a dCas9 molecule fused to Mxi1 or fragment thereof. In one embodiment, Mxi1 is fused to the C-terminus of the dCas9 molecule.

### Activators of Gene Expression

In some embodiments, the modulator of gene expression comprises a activator of gene expression. The activator may be any known activator of gene expression, for example, a VP16 activation domain, a VP64 activation domain, a p65 activation domain, an Epstein-Barr virus R transactivator Rta molecule, or fragment thereof. Activations that can be used with a dCas9 molecule are known in the art. See, e.g., Chavez et al., Nat Methods. (2016) 13:563-67, incorporated by reference herein in its entirety.

### VP16, VP64, VP 160

VP16 is a viral protein sequence of 16 amino acids that recruits transcriptional activators to promoters and enhancers. VP64 is a transcription activator comprising four copies of VP 16, e.g., a molecule comprising four tandem copies of VP16 connected by Gly-Ser linkers. VP160 is a transcription activator comprising 10 copies of VP16. In one embodiment, the methods and compositions disclosed herein include a fusion molecule comprising a dCas9 molecule fused to 1, 2, 3, 4, 5, 6, 7, 8, 9, 10, or more copies of VP16. In one embodiment, the methods and compositions disclosed herein include a fusion molecule comprising a dCas9 molecule fused to VP64. In one embodiment, the methods and compositions disclosed herein include a fusion molecule comprising a dCas9 molecule fused to VP 160. In one embodiment, VP64 is fused to the C-terminus, the N-terminus, or both the N-terminus and the C-terminus of the dCas9 molecule.

p65 activation domain (p65AD)

p65AD is the principal transactivation domain of the 65 kDa polypeptide of the nuclear form of the F-κB transcription factor. An exemplary sequence of human transcription factor p65 is available at the Uniprot database under accession number Q04206. In one embodiment, the methods and compositions disclosed herein include a fusion molecule comprising a dCas9 molecule fused to p65 or fragment thereof, e.g., p65AD.

### Epstein-Barr Virus (EBV) R Transactivator (Rta)

Rta, an immediate-early protein of EBV, is a transcriptional activator that induces lytic gene expression and triggers virus reactivation. In one embodiment, the methods and compositions disclosed herein include a fusion molecule comprising a dCas9 molecule fused to Rta or fragment thereof.

VP64, p65, Rta Fusions

In one embodiment, the methods and compositions disclosed herein include a fusion molecule comprising a dCas9 molecule fused to VP64, p65, Rta, or any combination thereof. The tripartite activator VP64-p65-Rta (also known as VPR), in which the three transcription activation domains are fused using short amino acid linkers, can effectively up-regulate target gene expression when fused to a dCas9 molecule. In one embodiment, the methods and compositions disclosed herein include a fusion molecule comprising a dCas9 molecule fused to VPR.

### Synergistic Activation Mediators (SAM)

In one embodiment, the methods and compositions disclosed herein include a CRISPR-Cas system that comprises three components: (1) a dCas9-VP64 fusion, (2) a gRNA incorporating two MS2 RNA aptamers at the tetraloop and stem-loop, and (3) the MS2-P65-HSF1 activation helper protein. This system, named Synergistic Activation Mediators (SAM), brings together three activation domains-VP64, P65 and HSF1 and has been described in Konermann et al., Nature. 2015; 517:583-8, incorporated by reference herein in its entirety.

### Ldb1 Self-Association Domain

In one embodiment, the methods and compositions disclosed herein include a fusion molecule comprising a dCas9 molecule fused to Ldb1 self-association domain. Ldb1 self-association domain recruits enhancer-associated endogenous Ldb1.

### Modulators of Epigenetic Modification

In one embodiment, the methods and compositions disclosed herein include a fusion molecule comprising a dCas9 molecule fused to a modulator of gene expression. In some embodiments, the modulator of gene expression comprises a modulator of epigenetic modification. In one embodiment, the fusion molecule modulates target gene expression via epigenetic modification, e.g., via histone acetylation or methylation, or DNA methylation, at a regulatory element of target gene, e.g., a promoter, enhancer or transcription start site. The modulator may be any known modulator of epigenetic modification, e.g., a histone acetyltransferase (e.g., p300 catalytic domain), a histone deacetylase, a histone methyltransferase (e.g., SUV39H1 or G9a (EHMT2)), a histone demethylase (e.g., LSD1), a DNA methyltransferase (e.g., DNMT3a or DNMT3a-DNMT3L), a DNA demethylase (e.g., TET1 catalytic domain or TDG), or fragment thereof.

### Histone Modification Activity

In some embodiments, the modulator of epigenetic modification may have histone modification activity. Histone modification activity may include but are not limited to histone deacetylase, histone acetyltransferase, histone demethylase, or histone methyltransferase activity.

In some embodiments, the modulator of epigenetic modification may have histone acetyltransferase activity. The histone acetyltransferase may be p300 or CREB-binding protein (CBP) protein, or fragments thereof. In some embodiments, the methods and compositions disclosed herein include a fusion molecule comprising a dCas9 molecule fused to acetyltransferase p300 or fragment thereof, e.g., the catalytic core of p300. In some embodiments, the methods and compositions disclosed herein include a fusion molecule comprising a dCas9 molecule fused to CREB-binding protein (CBP) protein or fragment thereof.

In some embodiments, the modulator of epigenetic modification may have histone demethylase activity. For example, the modulator of epigenetic modification may include an enzyme that removes methyl (CH3-) groups from nucleic acids or proteins (e.g., histones). In some embodiments, the methods and compositions disclosed herein include a fusion molecule comprising a dCas9 molecule fused to Lys-specific histone demethylase 1 (LSD1) or fragment thereof.

In some embodiments, the modulator of epigenetic modification may have histone methyltransferase activity. In some embodiments, the methods and compositions disclosed herein include a fusion molecule comprising a dCas9 molecule fused to SUV39H1 or fragment thereof. In some embodiments, the methods and compositions disclosed herein include a fusion molecule comprising a dCas9 molecule fused to G9a (EHMT2) or fragment thereof.

### DNA Demethylase Activity

In some embodiments, the modulator of epigenetic modification may have DNA demethylase activity. For example, the modulator of epigenetic modification may covert the methyl group to hydroxymethylcytosine as a mechanism for demethylating DNA. In some embodiments, the methods and compositions disclosed herein include a fusion molecule comprising a dCas9 molecule fused to Ten-eleven translocation methylcytosine dioxygenase 1 (TET1) or fragment thereof. In some embodiments, the methods and compositions disclosed herein include a fusion molecule comprising a dCas9 molecule fused to thymine DNA glycosylase (TDG) or fragment thereof.

### DNA Methylase Activity

In some embodiments, the modulator of epigenetic modification may have DNA methylase activity. For example, the modulator of epigenetic modification may have methylase activity which involves transferring a methyl group to DNA, RNA, proteins, small molecules, cytosine or adenine. In some embodiments, the methods and compositions disclosed herein include a fusion molecule comprising a dCas9 molecule fused to DNMT3A or fragment thereof. In some embodiments, the methods and compositions disclosed herein include a fusion molecule comprising a dCas9 molecule fused to DNMT3L or fragment thereof. In some embodiments, the methods and compositions disclosed herein include a fusion molecule comprising a dCas9 molecule fused to DNMT3L and DNMT3L or fragments thereof. In some embodiments, the methods and compositions disclosed herein include a fusion molecule comprising a dCas9 molecule fused to DNMT3A-DNMT3L fusion peptide.

DNMT3A:
(SEQ ID NO: 23)
MNHDQEFDPPKVYPPVPAEKRKPIRVLSLFDGIATGLLVLKDLGIQ
VDRYIASEVCEDSITVGMVRHQGKIMYVGDVRSVTQKHIQEWGPFD
LVIGGSPCNDLSIVNPARKGLYEGTGRLFFEFYRLLHDARPKEGDD
RPFFWLFENVVAMGVSDKRDISRFLESNPVMIDAKEVSAAHRARYF
WGNLPGMNRPLASTVNDKLELQECLEHGRIAKFSKVRTITTRSNSI
KQGKDQHFPVFMNEKEDILWCTEMERVFGFPVHYTDVSNMSRLARQ
RLLGRSWSVPVIRHLFAPLKEYFACV
DNMT3L:
(SEQ ID NO: 24)
MGPMEIYKTVSAWKRQPVRVLSLFRNIDKVLKSLGFLESGSGSGGG
TLKYVEDVTNVVRRDVEKWGPFDLVYGSTQPLGSSCDRCPGWYMFQ
FHRILQYALPRQESQRPFFWIFMDNLLLTEDDQETTTRFLQTEAVT
LQDVRGRDYQNAMRVWSNIPGLKSKHAPLTPKEEEYLQAQVRSRSK
LDAPKVDLLVKNCLLPLREYFKYFSQ NSLPL
DNMT3A-DNMT3L fusion peptide:
(SEQ ID NO: 27)
MNHDQEFDPPKVYPPVPAEKRKPIRVLSLFDGIATGLLVLKDLGIQ
VDRYIASEVCEDSITVGMVRHQGKIMYVGDVRSVTQKHIQEWGPFD
LVIGGSPCNDLSIVNPARKGLYEGTGRLFFEFYRLLHDARPKEGDD
RPFFWLFENVVAMGVSDKRDISRFLESNPVMIDAKEVSAAHRARYF
WGNLPGMNRPLASTVNDKLELQECLEHGRIAKFSKVRTITTRSNSI
KQGKDQHFPVFMNEKEDILWCTEMERVFGFPVHYTDVSNMSRLARQ
RLLGRSWSVPVIRHLFAPLKEYFACVSSGNSNANSRGPSFSSGLVP
LSLRGSHMGPMEIYKTVSAWKRQPVRVLSLFRNIDKVLKSLGFLES
GSGSGGGTLKYVEDVTNVVRRDVEKWGPFDLVYGSTQPLGSSCDRC
PGWYMFQFHRILQYALPRQESQRPFFWIFMDNLLLTEDDQETTTRF
LQTEAVTLQDVRGRDYQNAMRVWSNIPGLKSKHAPLTPKEEEYLQA
QVRSRSKLDAPKVDLLVKNCLLPLREYFKYFSQNSLPL

In one embodiment, the Cas9 fusion protein also comprises a nuclear localization sequence (NLS), e.g., a LS fused to the N-terminus and/or C-terminus of Cas9.

Nuclear localization sequences are known in the art. In one embodiment, the NLS comprises the amino acid sequence of SEQ ID NO: 25 or 26, a sequence substantially identical (e.g., at least 80%, 85%, 90%, 92%, 95%, 97%, 98%, 99% or higher identical) to SEQ ID NO: 25 or 26, or a sequence having one, two, three, four, five or more changes, e.g., amino acid substitutions, insertions, or deletions, relative to SEQ ID NO: 25 or 26, or any fragment thereof.

SEQ ID NO: 25 (exemplary nuclear localization
sequence):
APKKKRKVGIHGVPAA
SEQ ID NO: 26 (exemplary nuclear localization
sequence):
KRPAATKKAGQAKKKK

In some embodiments, the CRISPR/Cas9-based system may include a dCas9 molecule and a modulator of gene expression, or a nucleic acid encoding a dCas9 molecule and a modulator of gene expression. In one embodiment, the dCas9 molecule and the modulator of gene expression are linked covalently. In one embodiment, the modulator of gene expression is covalently fused to the dCas9 molecule directly. In one embodiment, the modulator of gene expression is covalently fused to the dCas9 molecule indirectly, e.g., via a non-modulator or linker, or via a second modulator. In one embodiment, the modulator of gene expression is at the N-terminus and/or C-terminus of the dCas9 molecule. In one embodiment, the dCas9 molecule and the modulator of gene expression are linked non-covalently. Exemplary sequences include but are not limited to those listed in Table 2. In some embodiments, the linker between the dCas9 and the at least one modulator of gene expression comprises an amino acid sequence corresponding to a linker listed in Table 2.

TABLE 2
Exemplary linker sequences

| | | SEQ |
|---|---|---|
| Description | Sequence | ID No: |
| linker | SSGNSNANSRGPSFSSGLVPLSLRGSH | 19 |
| XTEN80 | GGPSSGAPPPSGGSPAGSPTSTEEGTS | 20 |
| linker | ESATPESGPGTSTEPSEGSAPGSPAGS | |
| | PTSTEEGTSTEPSEGSAPGTSTEPSE | |
| XTEN16 | SGSETPGTSESATPES | 21 |
| linker | | |

In one embodiment, the dCas9 molecule is fused to a first tag, e.g., a first peptide tag. In one embodiment, the modulator of gene expression is fused to a second tag, e.g., a second peptide tag. In one embodiment, the first and second tag, e.g., the first peptide tag and the second peptide tag, non-covalently interact with each other, thereby bringing the dCas9 molecule and the modulator of gene expression into close proximity.

In one embodiment, the CRISPR/Cas9-based system includes a fusion molecule or a nucleic acid encoding a fusion molecule. In one embodiment, the fusion molecule comprises a sequence comprising a dCas9 fused to a modulator of gene expression. In one embodiment, the dCas9 molecule comprises a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 16511) dCas9 molecule, a Campylobacter lari (strain CF89-12) dCas9 molecule, a Streptococcus thermophilus (strain LMD-9) dCas9 molecule, or fragment thereof.

In one embodiment, the fusion molecule is a DNMT3A-DNMT3L (3A3L)-dCas9-KRAB fusion molecule comprising from the N-terminus to the C-terminus: a DNMT3A-DNMT3L fusion peptide (3A3L), a dCas9 peptide, and a KRAB peptide domain, fused directly or indirectly (e.g., via a linker).

In one embodiment, the fusion molecule is a DNMT3A-DNMT3L (3A3L)-dCas9-KRAB fusion molecule comprising from the N-terminus to the C-terminus: a DNMT3A-DNMT3L fusion peptide (3A3L), a dCas9 peptide, and a KRAB peptide domain, fused directly or indirectly (e.g., via a linker).

In one embodiment, the fusion molecule comprises the fusion molecule comprises the amino acid sequence of SEQ ID NO: 28, a sequence substantially identical (e.g., at least 80%, 85%, 90%, 91%, 92%, 93%, 94%, 95%, 96%, 97%, 98%, 99% or higher in sequence identity) to SEQ ID NO: 28, or a sequence having one, two, three, four, five or more changes, e.g., substitutions, insertions, or deletions, relative to SEQ ID NO: 28, or any fragment thereof.

DNMT3A-DNMT3L(3A3L)-dCas9-KRAB
(SEQ ID NO: 28)
MNHDQEFDPPKVYPPVPAEKRKPIRVLSLFDGIATGLLVLKDLGIQVDR
YIASEVCEDSITVGMVRHQGKIMYVGDVRSVTQKHIQEWGPFDLVIGGS
PCNDLSIVNPARKGLYEGTGRLFFEFYRLLHDARPKEGDDRPFFWLFEN
VVAMGVSDKRDISRFLESNPVMIDAKEVSAAHRARYFWGNLPGMNRPLA
STVNDKLELQECLEHGRIAKFSKVRTITTRSNSIKQGKDQHFPVEMNEK
EDILWCTEMERVFGFPVHYTDVSNMSRLARQRLLGRSWSVPVIRHLFAP
LKEYFACVSSGNSNANSRGPSFSSGLVPLSLRGSHMGPMEIYKTVSAWK
RQPVRVLSLFRNIDKVLKSLGFLESGSGSGGGTLKYVEDVTNVVRRDVE
KWGPFDLVYGSTQPLGSSCDRCPGWYMFQFHRILQYALPRQESQRPFFW
IFMDNLLLTEDDQETTTRFLQTEAVTLQDVRGRDYQNAMRVWSNIPGLK
SKHAPLTPKEEEYLQAQVRSRSKLDAPKVDLLVKNCLLPLREYFKYFSQ
NSLPLGGPSSGAPPPSGGSPAGSPTSTEEGTSESATPESGPGTSTEPSE
GSAPGSPAGSPTSTEEGTSTEPSEGSAPGTSTEPSEPKKKRKVMDKKYS
IGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKE
IFFDQSKNGYAGYIDGGASQEEFYKFIKPILEKMDGTEELLVKLNREDL
LRKQRTFDNGSIPHQIHLGELHAILRRQEDFYPFLKDNREKIEKILTFR
IPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKE
LGSQILKEHPVENTQLQNEKLYLYYLQNGRDMYVDQELDINRLSDYDVD
AIVPQSFLKDDSIDNKVLTRSDKNRGKSDNVPSEEVVKKMKNYWRQLLN
AKLITQRKFDNLTKAERGGLSELDKAGFIKRQLVETRQITKHVAQILDS
RMNTKYDENDKLIREVKVITLKSKLVSDFRKDFQFYKVREINNYHHAHD
AYLNAVVGTALIKKYPKLESEFVYGDYKVYDVRKMIAKSEQEIGKATAK
YFFYSNIMNFFKTEITLANGEIRKRPLIETNGETGEIVWDKGRDFATVR
KVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFL
EAKGYKEVKKDLIIKLPKYSLFELENGRKRMLASAGELQKGNELALPSK
YVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDT
TIDRKRYTSTKEVLDATLIHQSITGLYETRIDLSQLGGDAYPYDVPDYA
SLGSGSPKKKRKVEDPKKKRKVDGSGSETPGTSESATPESRTLVTFKDV
FVDFTREEWKLLDTAQQIVYRNVMLENYKNLVSLGYQLTKPDVILRLEK
GEEP
gRNA

As used herein, the term “guide sequence” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequence may form a duplex with a target sequence. The duplex may be a DNA duplex, an RNA duplex, or a RNA/DNA duplex. The terms “guide molecule” and “guide RNA” and “single guide RNA” are used interchangeably herein to refer to RNA-based molecules that are capable of forming a complex with a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA specifically encompasses RNA-based molecules having one or more chemically modifications (e.g., by chemical linking two ribonucleotides or by replacement of one or more ribonucleotides with one or more deoxyribonucleotides), as described herein.

The guide molecule or guide RNA of a CRISPR-Cas protein may comprise a tracr-mate sequence (encompassing a “direct repeat” in the context of an endogenous CRISPR system) and a guide sequence (also referred to as a “spacer” in the context of an endogenous CRISPR system). In some embodiments, the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence. In some embodiments, the guide molecule or sgRNA comprises a tracr sequence as set forth in SEQ ID No: 59.

An exemplary tracr sequence:
gttttagagctaGAAAtagcaagttaaaataaggctagtccgttatcaacttgaaaaagtggcaccgagtcggtgc (SEQ ID No: 59)

In general, a CRISPR-Cas system is characterized by elements that promote the formation of a CRISPR complex at the site of a target sequence. In the context of formation of a CRISPR complex, “target sequence” refers to a sequence to which a guide sequence is designed to have complementarity, where hybridization between a target DNA sequence and a guide sequence promotes the formation of a CRISPR complex.

In certain embodiments, the guide sequence or spacer length of the guide molecules is 15 to 50 nucleotides in length. In certain embodiments, the spacer length of the guide RNA is at least 15 nucleotides in length. In certain embodiments, the spacer length is from 15 to 17 nucleotides in length, from 17 to 20 nucleotides in length, from 20 to 24 nucleotides in length, from 23 to 25 nucleotides in length, from 24 to 27 nucleotides in length, from 27-30 nucleotides in length, from 30-35 nucleotides in length, or greater than 35 nucleotides in length.

In some embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

As described above, the CRISPR/Cas9 system utilizes gRNA that provides the targeting of the CRISPR/Cas9-based system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to cleave the target nucleic acid.

The term “target region”, “target sequence” or “protospacer” as used interchangeably herein refers to the region of the target gene to which the CRISPR/Cas9-based system targets. The CRISPR/Cas9-based system may include at least one gRNA, wherein the gRNAs target different DNA sequences. The target DNA sequences may be overlapping. The target sequence or protospacer is followed by a PAM sequence at the 3′ end of the protospacer. Different Type II systems have differing PAM requirements. For example, the S. pyogenes Type II system uses an “NGG” sequence, where “N” can be any nucleotide.

In some embodiments, the number of gRNA administered to the cell may be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 19 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs.

In some embodiments, the number of gRNAs administered to the cell may be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

In some embodiments, the gRNA is selected to increase or decrease transcription of a target gene. In some embodiment, the gRNA targets a region upstream of the transcription start site (TSS) of a target gene (e.g. VEGF), e.g., between 0-1000 bp upstream of the transcription start site of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp upstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the target gene. In one embodiment, the gRNA targets a region 0-300 bp upstream of the TSS of the target gene.

In some embodiments, the gRNA targets a region downstream of the transcription start site of a target gene, e.g., between 0-1000 bp downstream of the transcription start site of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp downstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the target gene. In one embodiment, the gRNA targets a region 0-300 bp downstream of the TSS of the target gene.

### VEGF

As used herein, the term “VEGF” refers to vascular endothelial growth factor. The VEGF pathway is involved in multiple aspects of vascular development and involves a family of proteins acting as angiogenic activators, including VEGF-A, VEGF-B, VEGF-C, VEGF-E and their respective receptors. VEGF-A, also referred to as VEGF or vascular permeability factor (VPF) is the target of anti-angiogenic therapy. VEGF-A exists in five isoforms that arise from alternative splicing of mRNA of a single VEGF gene: VEGFm, VEGF45, VEGFies, VEGF189 and VEGF206.

Human VEGFA has a cytogenetic location of 6p21.1 and the genomic coordinates are on Chromosome 6 on the forward strand at position 43,770,209-43,786,487. An example sequence of Human VEGF can be found at NCBI gene ID of 7422, and Ensembl Gene ID of ENSG00000112715. VEGFA induces proliferation and migration of vascular endothelial cells, and is essential for both physiological and pathological angiogenesis. Disruption of this gene in mice resulted in abnormal embryonic blood vessel formation.

In some embodiments, the method shows a significant decrease in VEGFA expression levels after transfection with the CRISPR-Cas9 system as disclosed herein. In some embodiments, compared to a control (e.g. transfection with a non-VEGFA targeting sgRNA), the decrease in VEGF expression level is about 80% or more, about 85% or more, about 90% or more, or about 95% or more.

In some embodiments, the decrease in VEGFA expression levels is retained at least about 96 hours, at least about one week, at least about 2 weeks, at least about 3 weeks, at least about 4 weeks, at least 6 weeks, at least two months, or even longer after transfection with the CRISPR-Cas9 system as disclosed herein. In some embodiments, the decrease in VEGF levels is retained from about 1 week to about 4 weeks, about 1 week to about 3 weeks, about 1 week to about 2 weeks, about 2 weeks to about 4 weeks, about 2 weeks to about 3 weeks, or about 3 weeks to about 4 weeks.

In some embodiments, the decrease in VEGFA expression levels is based on comparison of a baseline or predetermined level of VEGFA. In some embodiments, the decrease in VEGFA levels may be based on comparison of a first level of VEGF from a first sample from a subject to a second level of VEGF from a second sample from a subject.

The present disclosure provides sgRNA sequences that target a mouse or a rabbit VEGFA target gene. Exemplary sgRNAs include but are not limited to those listed in Table 3a and 3b. The present disclosure also provides sgRNA sequences that target human VEGFA (which also target homologous regions in monkey VEGFA). Exemplary sgRNAs include but are not limited to those listed in Table 4.

TABLE 3a
Exemplary Mouse VEGFA sgRNA sequences

| | | SEQ |
|---|---|---|
| Description | Sequence | ID No: |
| mouse VEGFA sgRNA1 | TCAGCTCGCCCCCAGTGCCG | 29 |
| mouse VEGFA sgRNA2 | GCAGCGAGGCCGCGGCACTG | 30 |
| mouse VEGFA sgRNA3 | GGGGCAGCCGAGCTGCAGCG | 31 |
| mouse VEGFA sgRNA4 | ACTGGGGGCGAGCTGAGCGG | 32 |
| mouse VEGFA sgRNA5 | CAGCGAGGCCGCGGCACTGG | 33 |
| mouse VEGFA sgRNA6 | GCCGAGCTGCAGCGAGGCCG | 34 |
| mouse VEGFA sgRNA7 | GCCGCGGCCTCGCTGCAGCT | 35 |

TABLE 3b
Exemplary Rabbit VEGFA sgRNA sequences

| | | SEQ |
|---|---|---|
| Description | Sequence | ID No: |
| Rabbit VEGFA sgRNA1 | TGGAACCCTGGAGTGACCCC | 60 |
| Rabbit VEGFA sgRNA2 | GCCCAGAGCTGTTGGAACCC | 61 |
| Rabbit VEGFA sgRNA3 | GGAGGCCGGGGGTCACTCCA | 62 |
| Rabbit VEGFA sgRNA4 | GGAGCCCGTCAGGGACGGGT | 63 |
| Rabbit VEGFA sgRNA5 | TAGGGGGCGAGGGTGCTGCG | 64 |
| Rabbit VEGFA sgRNA6 | CTCCAGGGTTCCAACAGCTC | 65 |
| Rabbit VEGFA sgRNA7 | TGTGTGAGCGCGTGTGTAGG | 66 |
| Rabbit VEGFA sgRNA8 | AGGGGGCGAGGGTGCTGCGT | 67 |
| Rabbit VEGFA sgRNA9 | GGGAAGGGGCCCAGAGCTGT | 68 |
| Rabbit VEGFA sgRNA10 | GCGGGGAGCCCGTCAGGGAC | 69 |
| Rabbit VEGFA sgRNA11 | GTCCCTGACGGGCTCCCCGC | 70 |
| Rabbit VEGFA sgRNA12 | AGGAGGCCGGGGGTCACTCC | 71 |
| Rabbit VEGFA sgRNA13 | GCTGCGTGGGGAGGAGGCCGG | 72 |
| Rabbit VEGFA sgRNA14 | TCCAGGGTTCCAACAGCTCT | 73 |
| Rabbit VEGFA sgRNA15 | GACGGGCGGGGAGCCCGTCA | 74 |
| Rabbit VEGFA sgRNA16 | GTGAGGAGCGCAGAGGCTTG | 75 |
| Rabbit VEGFA sgRNA17 | GGGAGCGGTGAGGAGCGCAG | 76 |
| Rabbit VEGFA sgRNA18 | TCAGTCGCCTGGGAGCGGTG | 77 |
| Rabbit VEGFA sgRNA19 | GGGGCAGCCGGGCTGCGAGG | 78 |
| Rabbit VEGFA sgRNA20 | GGTGAGGAGCGCAGAGGCTT | 79 |
| Rabbit VEGFA sgRNA21 | CGCAGGGGGAGCGGAGCCGG | 80 |
| Rabbit VEGFA sgRNA22 | GCGAGGAGGCCGCGGCGCAG | 81 |
| Rabbit VEGFA sgRNA23 | CGAGGAGGCCGCGGCGCAGG | 82 |
| Rabbit VEGFA sgRNA24 | GCTCCGCTCCCCCTGCGCCG | 83 |
| Rabbit VEGFA sgRNA25 | CTGCGCTCCTCACCGCTCCC | 84 |

TABLE 4
Exemplary Human VEGFA sgRNA sequences

| | | SEQ |
|---|---|---|
| Description | Sequence | ID No: |
| human VEGFA sgRNA1 | AGCACCAGCGCTCTGTCGGG | 36 |
| human VEGFA sgRNA2 | GGGGCAGCCGGGTAGCTCGG | 37 |
| human VEGFA sgRNA3 | GGCTAGCACCAGCGCTCTGT | 38 |
| human VEGFA sgRNA4 | GCTAGCACCAGCGCTCTGTC | 39 |
| human VEGFA sgRNA5 | GCCGGGTAGCTCGGAGGTCG | 40 |
| human VEGFA sgRNA6 | GCTCGGAGGTCGTGGCGCTG | 41 |
| human VEGFA sgRNA7 | GCGCTCTGTCGGGAGGCGCA | 42 |
| human VEGFA sgRNA8 | AGCTCGGAGGTCGTGGCGCT | 43 |
| human VEGFA sgRNA9 | CGCTCTGTCGGGAGGCGCAG | 44 |
| human VEGFA sgRNA10 | CTCGGAGGTCGTGGCGCTGG | 45 |
| human VEGFA sgRNA11 | TAGCTCGGAGGTCGTGGCGC | 46 |
| human VEGFA sgRNA12 | GCCACGACCTCCGAGCTACC | 47 |
| human VEGFA sgRNA13 | CGGTTAGGTGGACCGGTCAG | 48 |
| human VEGFA sgRNA14 | GGGGCGGATGGGTAATTTTC | 49 |
| human VEGFA sgRNA15 | GGGAAGCTCGACCCCCACCA | 50 |
| human VEGFA sgRNA16 | GTCGAGCTTCCCCTTCATTG | 51 |
| human VEGFA sgRNA17 | GAGCTTCCCCTTCATTGCGG | 52 |
| human VEGFA sgRNA18 | GCCCGGGCCCGAGCCGCGTG | 53 |
| human VEGFA sgRNA19 | GCTGGTAGCGGGGAGGATCG | 54 |
| human VEGFA sgRNA20 | GGTAGCGGGGAGGATCGCGG | 55 |
| human VEGFA sgRNA21 | GGGGAGGATCGCGGAGGCTT | 56 |
| human VEGFA sgRNA22 | GGGAGGATCGCGGAGGCTTG | 57 |
| human VEGFA sgRNA23 | GGACCGGTCAGCGGACTCAC | 58 |

In one embodiment, the gRNA targets a promoter region of a target gene. In one embodiment, the gRNA targets an enhancer region of a target gene. gRNA can be divided into a target binding region, a Cas9 binding region, and a transcription termination region. The target binding region hybridizes with a target region in a target gene. Methods for designing such target binding regions are known in the art, see, e.g., Doench et al., Nat Biotechnol. (2014) 32:1262-7; and Doench et al., Nat Biotechnol. (2016) 34:184-91, incorporated by reference herein in their entirety. Design tools are available at, e.g., Feng Zhang lab's target Finder, Michael Boutros lab's Target Finder (E-CRISP), RGEN Tools (Cas-OF Finder), CasFinder, and CRISPR Optimal Target Finder. In certain embodiments, the target binding region can be between about 15 and about 50 nucleotides in length (about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides in length). In certain embodiments, the target binding region can be between about 19 and about 21 nucleotides in length. In one embodiment, the target binding region is 15, 16, 17, 18, 19, 20, 21, 22, 23, 24, or 25 nucleotides in length.

In one embodiment, the target binding region is complementary, e.g., completely complementary, to the target region in the target gene. In one embodiment, the target binding region is substantially complementary to the target region in the target gene. In one embodiment, the target binding region comprises no more than 1, 2, 3, 4, 5, 6, 7, 8, 9, or 10 nucleotides that are not complementary to the target region in the target gene.

In one embodiment, the target binding region is engineered to improve stability or extend half-life, e.g., by incorporating a non-natural nucleotide or a modified nucleotide in the target binding region, by removing or modifying an RNA destabilizing sequence element, by adding an RNA stabilizing sequence element, or by increasing the stability of the Cas9/gRNA complex. In one embodiment, the target binding region is engineered to enhance its transcription. In one embodiment, the target binding region is engineered to reduce secondary structure formation. In one embodiment, the Cas9 binding region of gRNA is modified to enhance the transcription of the gRNA. In one embodiment, the Cas9 binding region of gRNA is modified to improve stability or assembly of the Cas9/gRNA complex.

### Delivery Systems

The present disclosure also provides delivery systems for introducing components of the systems and compositions herein to cells, tissues, organs, or organisms. A delivery system may comprise one or more delivery vehicles and/or cargos.

### Cargos

The delivery systems may comprise one or more cargos. The cargos may comprise one or more components of the systems and compositions herein. A cargo may comprise one or more of the following: i) a plasmid encoding one or more Cas proteins; ii) a plasmid encoding one or more guide RNAs, iii) mRNA of one or more Cas proteins; iv) one or more guide RNAs; v) one or more Cas proteins; vi) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516.

### Physical Delivery

In some embodiments, the cargos may be introduced to cells by physical delivery methods. Examples of physical methods include microinjection, electroporation, and hydrodynamic delivery.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present disclosure may a greatest dimension (e.g. diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

### Vectors

The systems, compositions, and/or delivery systems may comprise one or more vectors. The present disclosure also include vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 1 1d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC).

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

### Regulatory Elements

A vector may comprise one or more regulatory elements. The regulatory element(s) may be operably linked to coding sequences of Cas proteins, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof.

Examples of pol III promoters include, but are not limited to, U6 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the -actin promoter, the phosphoglycerol kinase (PGK) promoter, and the EF1a promoter.

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno-Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82:5887-5911 (2008)) and WO 2021/183807A1, which are incorporated by reference herein in their entirety.

CRISPR-Cas AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin 0, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

LNPs can be easily prepared by various methods as known in the art, e.g. by mixing the organic phase and the water phase. The mixing of the two phases can be achieved by microfluidic device and impinging stream reactors. The more adequate the organic phase and the water phase are mixed, the better the embedding rate and particle size distribution of LNP could be obtained. Preferably, the particle size of the LNP can be adjusted by changing the mixing speed of the organic phase and the water phase. The faster the mixing speed, the smaller the particle size of the LNP would be prepared. The embedding efficiency can be optimized by regulating the N/P ratio of the LNP system. In some preferable embodiments, the N/P ratio is 1:1-9:1.

In some examples LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

In some embodiments, LNPs are used for delivering an mRNA and gRNAs (e.g. mRNA fusion molecule comprising DNMT3A-DNMT3L (3A-3L)-dCas9-KRAB and at least one sgRNA targeting VEGF.

Components of LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-In some embodiments, LNPs may comprise ionizable lipids. In some embodiments, ionizable lipids include but are not limited to pH-responsive ionizable lipids, thermal-responsive ionizable lipids and light-responsive ionizable lipids. In some embodiments, ionizable lipids include cationic lipids and anionic lipids that are ionized under the certain conditions, such as, but not limited to pH, temperature or light. In some embodiments, the molar ratio of ionizable lipids of the LNP is 20% to about 70% (e.g., about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 30% to about 70%, about 30% to about 65%, about 30% to about 60%, about 30% to about 55%, about 30% to about 50%, about 30% to about 45%, about 30% to about 40%, about 30% to about 35%, about 40% to about 70%, about 40% to about 65%, about 40% to about 60%, about 40% to about 55%, about 40% to about 50%, about 40% to about 45%, about 50% to about 70%, about 50% to about 65%, about 50% to about 60%, about 50% to about 55%, about 60% to about 70%, or about 60% to about 65%)

In some embodiments, LNPs may comprise PEGylated lipids. In some embodiments, the molar ratio of PEGylated lipids of the LNP is 0% to about 30% (e.g., about 0% to about 30%, about 0% to about 25%, about 0% to about 20%, about 0% to about 15%, about 0% to about 10%, about 10% to about 30%, about 10% to about 25%, about 10% to about 20%, about 10% to about 15%, about 20% to about 30%, or about 20% to about 25%).

In some embodiments, LNPs may comprise supporting lipids. In some embodiments, the molar ratio of supporting lipids of the LNP is 30% to about 50% (e.g. about 30% to about 50%, about 30% to about 45%, about 30% to about 40%, about 30% to about 35%, about 40% to about 50%, or about 40% to about 45%)

In some embodiments, LNPs may comprise cholesterol. In some embodiments, the molar ratio of cholesterol of the LNP is 10% to about 50% (e.g., about 10% to about 50%, about 10% to about 45%, about 10% to about 40%, about 10% to about 35%, about 10% to about 30%, about 10% to about 25%, about 10% to about 20%, about 10% to about 15%, about 20% to about 50%, about 20% to about 45%, about 20% to about 40%, about 20% to about 35%, about 20% to about 30%, about 20% to about 25%, about 30% to about 50%, about 30% to about 45%, about 30% to about 40%, about 30% to about 35%, about 40% to about 50% or about 40% to about 45%).

In some embodiments, LNPs may comprise a mixture of ionizable lipids (20%-70%, molar ratio), PEGylated lipids (0%-30%, molar ratio), supporting lipids (30%-50%, molar ratio), and cholesterol (10%-50%, molar ratio).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-Other lipids

The lipid particles may also comprise one or more other types of lipids, e.g., cationic lipids, such as amino lipid 2,2-dilinoleyl-4-dimethylaminoethyl-Lipoplexes and/or polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca2+ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nano-sized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus I (HIV-I). Examples of CPPs include to Penetratin, Tat (48-60), Transportan, and (R-AhX-R4) (Ahx refers to aminohexanoyl). Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

### DNA Nanoclews

In some embodiments, the delivery vehicles comprise DNA nanoclews. A DNA nanoclew refers to a sphere-like structure of DNA (e.g., with a shape of a ball of yam). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136 (42): 14722-5; and Sun Wet al, Angew Chem Int Ed Engl. 2015 Oct. 5; 54 (41): 12029-33. DNA nanoclew may have a palindromic sequences to be partially complementary to the gRNA within the Cas: gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas: gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNATM) constructs, and those described in Mout R, et al. (2017). ACS Nano 11:2452-8; Lee K, et al. (2017). Nat Biomed Eng 1:889-901.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D′Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage SS et al., Synthetic mRNA expressed Cas13a mitigates RNA virus infections, www.biorxiv.org/content/10. 1 101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi: 10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci US A 98:3185-90; Teng K W, et al. (2017). Elife 6: e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Ace Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates Kand Kostarelos K. (2013). Adv Drug Deliv Rev 65:2023-33.), bare mesoporous silica nanoparticles (MSNPs) (e.g., as described in Luo GF, et al. (2014). Sci Rep 4:6064), and dense silica nanoparticles (SiNPs) (as described in Luo D and Saltzman W M. (2000). Nat Biotechnol 18:893-5).

### Methods of Use

The compositions and systems herein may be used for a variety of applications, including modifying non-animal organisms such as plants and fungi, and modifying animals, treating and diagnosing diseases in plants, animals, and humans. In general, the compositions and systems may be introduced to cells, tissues, organs, or organisms, where they modify the expression and/or activity of one or more genes.

### Cells and Organisms

The present disclosure provides cells, tissues, organisms comprising the engineered Cas protein, the CRISPR-Cas systems, the polynucleotides encoding one or more components of the CRISPR-Cas systems, and/or vectors comprising the polynucleotides. The disclosure also provides for the nucleotide sequence encoding the effector protein being codon optimized for expression in a eukaryote or eukaryotic cell in any of the herein described methods or compositions. In an embodiment of the disclosure, the codon optimized effector protein is any Cas protein discussed herein and is codon optimized for operability in a eukaryotic cell or organism, e.g., such cell or organism as elsewhere herein mentioned, for instance, without limitation, a yeast cell, or a mammalian cell or organism, including a mouse cell, a rat cell, and a human cell or non-human eukaryote organism, e.g., plant.

In certain embodiments, the modification of the target locus of interest may result in: the eukaryotic cell comprising altered expression of at least one gene product; the eukaryotic cell comprising altered expression of at least one gene product, wherein the expression of the at least one gene product is increased; the eukaryotic cell comprising altered expression of at least one gene product, wherein the expression of the at least one gene product is decreased; or the eukaryotic cell comprising an edited genome.

In certain embodiments, the eukaryotic cell may be a mammalian cell or a human cell.

In further embodiments, the non-naturally occurring or engineered compositions, the vector systems, or the delivery systems as described in the present specification may be used for: site-specific gene knockout; site-specific genome editing; RNA sequence-specific interference; or multiplexed genome engineering.

Also provided is a gene product from the cell, the cell line, or the organism as described herein. In certain embodiments, the amount of gene product expressed may be greater than or less than the amount of gene product from a cell that does not have altered expression or edited genome. In certain embodiments, the gene product may be altered in comparison with the gene product from a cell that does not have altered expression or edited genome.

### Exemplary Therapies

The present disclosure provides a use of the CRISPR-Cas system for treatment in a variety of diseases and disorders. In some embodiments, the disclosure described herein relates to a method for therapy in which cells are edited ex vivo by CRISPR or the base editor to modulate VEGF (e.g. VEGFA) gene, with subsequent administration of the edited cells to a patient in need thereof. In some embodiments, the editing involves knocking in, knocking out or knocking down expression of VEGF (e.g. VEGFA) gene in a cell.

In some embodiments, the VEGFA targeting CRISPR-Cas system as described herein are useful for inhibiting cellular processes that are mediated through VEGFA, and have indications for prophylaxis or therapy of disorders associated with aberrant angiogenesis and/or lymphangiogenesis (e.g., various ocular disorders and cancer) that is stimulated by the actions of VEGFA or VEGFA related receptors.

The VEGFA targeting CRISPR-Cas system as described herein, including the fusion molecule comprising a least one DNA binding protein and at least one modulator of gene expression or a nucleic acid sequence encoding the fusion molecule, and sgRNAs designed to target the DNA sequence near the VEGF gene and/or within a VEGF regulatory element, are therapeutically useful for treating or preventing any disease of condition which is improved, ameliorated, inhibited or prevented by the removal, inhibition or reduction of VEGF-A.

A non-exhaustive list of specific conditions improved by inhibition or reduction of VEGFA include: clinical conditions that are characterized by excessive vascular endothelial cell proliferation, vascular permeability, edema or inflammation such as brain edema associated with injury, stroke or tumor; edema associated with inflammatory disorders such as psoriasis or arthritis, including rheumatoid arthritis; asthma; generalized edema associated with burns; ascites and pleural effusion associated with tumors, inflammation or trauma; chronic airway inflammation; capillary leak syndrome; sepsis; kidney disease associated with increased leakage of protein; and eye disorders such as age related macular degeneration and diabetic retinopathy.

A “neovascular disorder” is a disorder or disease state characterized by altered, dysregulated or unregulated angiogenesis. Examples of neovascular disorders include neoplastic transformation (e.g. cancer) and ocular neovascular disorders including diabetic retinopathy and age-related macular degeneration.

An “ocular neovascular disorder” is a disorder characterized by altered, dysregulated or unregulated angiogenesis in the eye of a patient. Such disorders include optic disc neovascularization, iris neovascularization, retinal neovascularization, choroidal neovascularization, corneal neovascularization, vitreal neovascularization, glaucoma, pannus, pterygium, macular edema, diabetic retinopathy, diabetic macular edema, vascular retinopathy, retinal degeneration, uveitis, inflammatory diseases of the retina, and proliferative vitreoretinopathy.

In some embodiments, the disease to be treated by the composition and method as disclosed herein is associated with angiogenesis (the formation of blood vessels). In some embodiments, the disease is neovascular disorder, such as an ocular neovascular disorder, including Age related macular degeneration (AMD), including dry AMD, wet AMD.

### Examples

### Example 1: Fusion Molecule Plasmid Construction and Knock Down Efficiency

Two plasmids were constructed to form the “EPICAS” system (FIG. 1A). The “fusion molecule” or “catalytic protein” plasmid encodes dCas9, DNMT3A, DNMT3L and KRAB peptides. A fused DNMT3A and DNMT3L (3A3L) peptide is at the N-terminal of dCas9, and KRAB is at the C-terminal of dCas9. Thus, the fusion molecule has a 3A3L-dCas9-KRAB, from the N-terminal to the C-terminal end. The “sgRNA” plasmid encodes a sgRNA sequence that targets the VEGFa gene. The “scaffold” is the sequence of the gene or promoter of VEGFA gene. Multiple sgRNAs were designed to target the region within the 250 bp upstream and downstream of the transcription start site (TSS) of the mouse VEGFa gene. Specifically, 7 sgRNAs (SEQ ID Nos: 29-35) were designed and corresponding sgRNA plasmids were generated for subsequent transfection.

Individual sgRNA plasmids were co-transfected with the catalytic protein plasmid into the mouse N2A cell lines (National collection of Authenticated Cell cultures). After 48 hours, the top 10% GFP+ and mCherry+ cells were sorted by FACS. RT-QPCR experiments were performed to evaluate the mRNA expression level of VEGFa. All 7 sgRNAs that were tested showed significantly down-regulated expression of VEGF in N2A cells and showed a knock-down efficiency of about 80%. Among them, cells transfected with sgRNA3, sgRNA5 and sgRNA7 showed the best knock-down effect, reaching about 84% (FIG. 1B).

Next, sgRNA3, sgRNA4 and sgRNA5 were selected to further test the retaining of knock-down effect in a longer period. sgRNA plasmids were co-transfected with the catalytic protein plasmid into the mouse N2A cell lines. After 48 hours, the top 10% GFP+ and mCherry+ cells were sorted by FACS and continue to be cultured. After one week, the cells were collected and RT-QPCR experiments were performed to evaluate the mRNA expression level of VEGFa. The result indicated that the gene silencing effect was retained and even improved compared to that after 48 hours of transfection, reaching more than 90% (FIG. 1C). It is speculated that VEGFa mRNA has a relatively long half-life in cell, and degradation of the existing VEGFa mRNA and the hindered synthesis of new VEGFa mRNA both contribute to the lower level of VEGFa mRNA after one week.

In addition, the combination of sgRNA3, sgRNA4 and sgRNA5 (sgMix) was tested to determine if the combination of more than one sgRNA could further reduce the gene expression level of VEGFa in N2A cells. The result showed sgMix significantly knocked down the expression level of VEGFa (FIG. 1C).

### Example 2: In vitro Transcription of mRNA Encoding Fusion Molecule

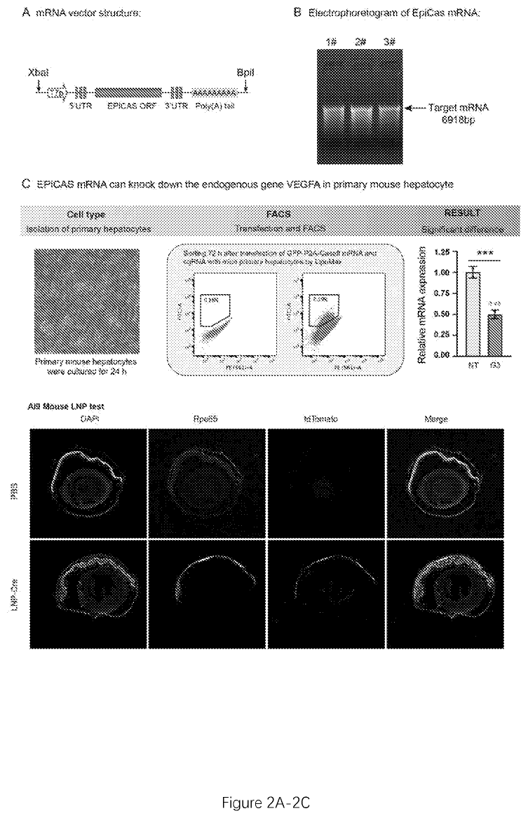

In vitro transcription and purification was used to produce mRNA corresponding to the fusion molecule or catalytic protein of the EPICAS system. First, a plasmid containing all of the fusion molecule elements, including a cassette of 5′UTR-DNMT3A-DNMT3L-dCas9-KRAB-3′UTR-poly A was constructed. The plasmid sequence was linearized by XbaI and BpiI restriction enzyme digestion (FIG. 2A). An in vitro transcription reaction containing linearized DNA template, T7 RNA polymerase, NTPs and cap analogue was performed to produce mRNA containing N1-methylpseudouridine. After digestion of the DNA template with DNase I, the mRNA product underwent purification and buffer exchange, and the purity of the final mRNA product was assessed with capillary gel electrophoresis (FIG. 2B). The 100-mer sgRNAs were chemically synthesized with minimal end-modifications under solid phase synthesis conditions by a commercial supplier (Genewiz).

To test the function of in vitro transcribed mRNAs, a Snrpn-GFP reporter system was constructed in HEK293T cells. The reporter system controls the expression of GFP using a synthetic methylation-sensing promoter (conserved sequence elements from the promoter of an imprinted gene, Snrpn). Insertion of this reporter construct into a genomic locus showed the methylation state of the adjacent sequences. Using Lipofectamine Messenger MAX, the in vitro transcribed mRNAs described above (GFP-P2A-Casoff mRNA) were co-transfected with sgRNA targeting the Snrpn gene into mouse primary hepatocytes (FIG. 2C, left panel). FACS analysis indicated that 72h after transfection of the mRNA and sgRNA, the proportion of GFP+ cells was significantly increased, suggesting the reporter system was successfully established in mouse hepatocytes (FIG. 2C, middle panel). Besides, the expression level of VEGFA mRNA at 72 h after transfection was obviously reduced (FIG. 2C, right panel).

Together, these results show that EPICAS mRNA can silence the expression of VEGFA for a long duration of time using transient transfection.

### Example 3: Lipid Nanoparticle Encapsulation of mRNA Encoding Fusion Molecules and sgRNAs

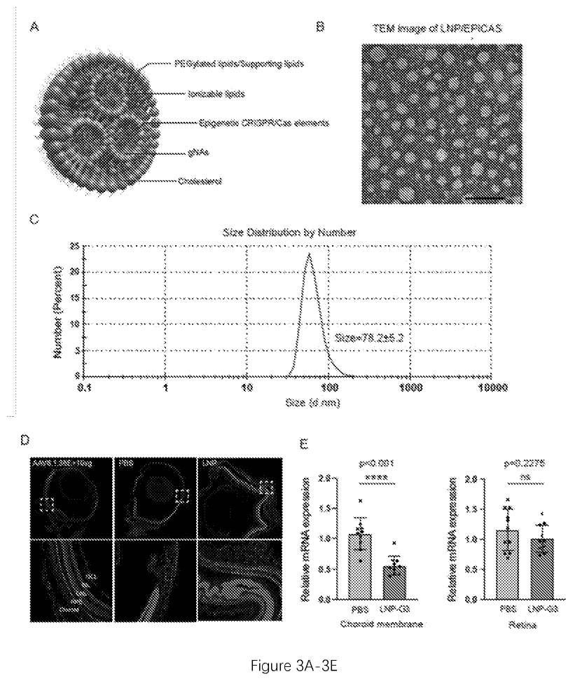

Standard methods known in the art were used to formulate LNPs for delivery of fusion molecule mRNA and sgRNA to mouse choroids. LNPs contained fusion molecule mRNA and sgRNA targeting VEGFA gene at a 1:1 ratio by weight (FIG. 3A). A mixture of ionizable lipids (49.5%, molar ratio), PEGylated lipids (2.5%, molar ratio), supporting lipids (DPPC) (9.9%, molar ratio), and cholesterol (38.1%, molar ratio), lipid nanoparticles (LNPs) were formulated using well-designed impinging stream reactors or microfluidic devices. By varying the proportion of ionizable lipids, the release kinetics of sgRNA and mRNA can be modified. With higher proportion of the ionizable lipids (molar ratio above 55%), sgRNA was released much faster than mRNA. Transmission electron microscope (TEM) images showed that the LNPs were spherical and nano-sized particles (FIG. 3B). LNPs had uniform-sizes (78.2±5.2 nm, PDI <0.10) using dynamic light scattering (NanoSZ, Malvern) (FIG. 3C).

To test whether the LNPs could deliver the mRNAs successfully to posterior retina and choroid regions in vivo, LNPs containing luciferase mRNAs were produced and administered into the eyes of Ai9 mouse (Jax Lab) by intravitreal injection. AAV8 virus expressing EF1A-Cre-GFP was used as a positive control and PBS was used as a negative control. The in vivo imaging result of the fluorescence indicated that, LNP could deliver mRNA to Retinal Pigment Epithelium (RPE) and choroid in a highly efficient manner (FIG. 3D). The positive control AAV8 could efficiently infect RPE and choroid.

### Example 4: VEGF Gene Silencing Using LNP Delivery of mRNA Encoding Fusion Molecules and sgRNAs in Mice

To test whether mRNAs and sgRNAs delivered by LNPs could successfully knock-down the level of VEGFa mRNAs, LNPs containing EPICAS mRNA and sgRNA3 were produced and administered into the eyes of Ai9 mouse by intravitreal injection. 5 days after the injection, the mice were euthanized and the retina and choroid were obtained and processed for mRNA purification. RT-QPCR experiments were performed to evaluate the mRNA expression level of VEGFa.

The result showed significantly down-regulated expression of VEGFa in choroid compared to the control PBS group (FIG. 3E), indicating the efficacy of EPICAS system in silencing VEGF gene expression in vivo. On the other hand, the reduction of VEGFa expression in retina was not significant compared to control group, indicating a potential preference for choroid in reducing gene expression.

### Example 5: Rabbit VEGF Gene Silencing in Rabbit Cells

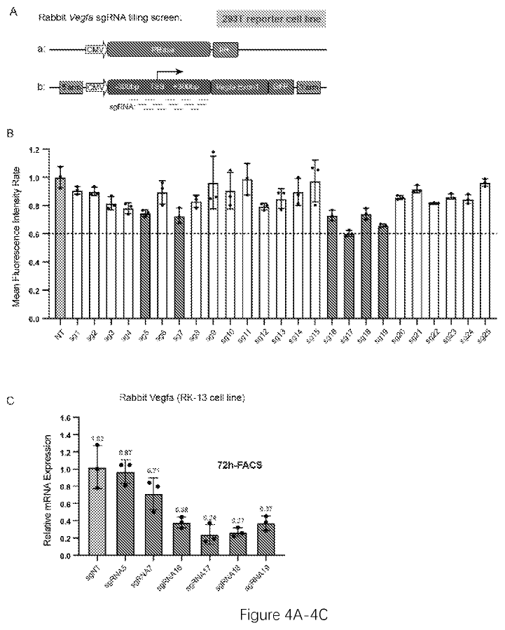

A reporter system for screening rabbit VEGFa sgRNA (SEQ ID Nos: 60-84) was established. An artificial sequence containing the 500 bp upstream of the transcription start site (TSS) to the first exon of the rabbit VEGFa gene and GFP at the C terminus of the first exon was constructed (FIG. 4A). The artificial sequence was integrated into the genome of 293T cells by using piggybac transpon system. A cell line stably transfected with GFP was obtained, which was used for sgRNA screening. In this experiment, The reporter cells were transfected with EPICAS plasmids and sgRNA and detected the fluorescence intensity of the reporter cells 72 hours later. Flow cytometry analysis results showed that the majority of sgRNA significantly decreased the intensity of GFP (FIG. 4B). Four out of six of the sgRNA that had good knockdown effects were transfected together with EPICAS plasmids into rabbit RK-13 cells and targeted the endogenous gene VEGFA in the rabbit cells. QPCR results showed that these sgRNA significantly reduced the mRNA expression of VEGFA in RK-13 cells (FIG. 4C).

### Example 6: Human VEGF Gene Silencing in Human Cell Lines

A reporter cell line was constructed in order to test the efficacy of VEGF gene silencing in human cell lines. A plasmid was constructed to have a CMV promoter driven cassette, where the cassette had the following elements in the 5′ to 3′ direction: 5′-pCMV-300 bp-TSS-+300 bp-VEGF exon1-2A-GFP-3′. In this reporter system, the CMV promoter drives the expression of VEGF and GFP fluorescence. If VEGF is silenced, the transcription of GFP is terminated. Together with PiggyBac transposase (PBase) plasmid, the reporter plasmid was transfected into the HEK293T cells. Cells with successful reporter cassette integration were sorted by FACS according to the expression of GFP fluorescence.

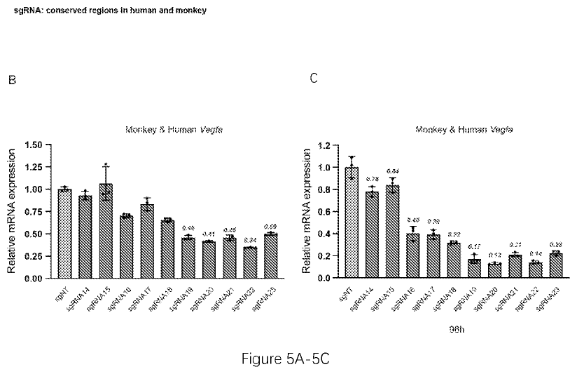

Multiple sgRNAs were designed to target the homologous region within the 300 bp upstream and downstream of the transcription start site (TSS) of the monkey and human VEGF gene (FIG. 5A). 23 sgRNAs (SEQ ID Nos: 36-58) were chosen for plasmid construction to encode each one of the sgRNAs. Individual sgRNA plasmids were co-transfected with the catalytic protein (DNMT3A-DNMT3L-dCas9-KRAB) plasmid into HEK293T cells. After 48 or 96 hours, GFP+ and mCherry+ double positive cells were sorted by FACS. RT-QPCR experiments were performed to evaluate the mRNA expression level of VEGFa.

Most sgRNAs that were tested showed significantly down-regulated expression of VEGFa in 293T cells (FIG. 5B). Cells transfected with sgRNA10, sgRNA19, sgRNA20, sgRNA21, sgRNA22 and sgRNA23 resulted in more than 50% down regulation of VEGFa after 48 hours. After 96 hours, the VEGFA expression level went even lower, with sgRNA19, sgRNA20 and sgRNA22 reaching more than 80% of down regulation.

Together, these results show that the EPICAS system successfully silenced the expression of VEGFA in both mouse cells and human cells with high efficiency and persistence supporting silencing VEGF gene expression by epigenetic editing. LNPs were successfully used to deliver the EPICAS system in vivo. Accordingly, LNP formulation of the EPICAS system can be used in the treatment of VEGF related diseases such as AMD.

1. A composition comprising a fusion molecule comprising a least one DNA binding protein and at least one modulator of gene expression, or a nucleic acid sequence encoding the fusion molecule, wherein the fusion molecule is targeted to a genomic region near a VEGF gene and/or within a VEGF regulatory element, the at least one modulator of gene expression provides a modification of at least one nucleotide near the VEGF gene and/or within a VEGF regulatory element, wherein the at least one modulator of gene expression comprises a DNA methyltransferase (DNMT), a DNA demethylase, a histone methyltransferase, a histone demethylase, or a portion thereof, or a zinc finger protein-based transcription factor or a portion thereof, or a combination thereof, and
wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

2. The composition of claim 2, wherein the VEGF gene is VEGF-A gene.

3. The composition of claim 2 or 3, wherein the VEGF regulatory element is a transcription start site, core promoter, a proximal promoter, a distal enhancer, a silencer, an insulator element, a boundary element or a locus control region.

4. The composition of any one of claims 1-3, wherein the modification of at least one nucleotide near the VEGF gene and/or within a VEGF regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the transcription start site of the VEGF gene.

5. The composition of any one of claims 1-3, wherein the modification of at least one nucleotide near the VEGF gene and/or within a VEGF regulatory element is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the transcription start site of the VEGF gene.

6. The composition of claim 4 or 5, wherein the modification of at least one nucleotide near the VEGF gene and/or within a VEGF regulatory element is located within 500 bp upstream of the transcription start site to 500 bp downstream of the transcription start site of the VEGF gene.

7. The composition of claim 6, wherein the modification of at least one nucleotide near the VEGF gene and/or within a VEGF regulatory element is located within 300 bp upstream of the transcription start site to 300 bp downstream of the transcription start site of the VEGF gene.

8. The composition of claim 4 or 5, wherein the modification of at least one nucleotide near the VEGF gene and/or within a VEGF regulatory element is located within 1000 bp upstream of the transcription start site to within 300 bp downstream of the transcription start site of the VEGF gene.

9. The composition of any of the preceding claims, wherein the modification of at least one nucleotide is a DNA methylation.

10. The composition of any of the preceding claims, wherein the at least one modulator of gene expression comprises one or more selected from a DNA methyltransferase (DNMT), a zinc-finger protein-based transcription factor, a portion thereof and any combinations thereof.

11. The composition of claim 10, wherein the at least one modulator of gene expression comprises a DNA methyltransferase or a portion thereof, and a zinc finger protein-based transcription factor or a portion thereof.

12. The composition of claim 10 or 11, wherein the DNA methyltransferase is DNMT3A, DNMT3B, DNMT3L, DNMT1 or DNMT2.

13. The composition of claim 12, wherein the DNMT3A comprises the amino acid sequence of SEQ ID NO: 23, and/or the DNMT3L comprises the amino acid sequence of SEQ ID NO: 24.

14. The composition of claim 10 or 11, wherein the zinc finger protein-based transcription factor is Kruppel-associated suppression box (KRAB).

15. The composition of claim 14, wherein the KRAB comprises the amino acid sequence of SEQ ID NO: 22.

16. The composition of claim 15, wherein the DNA methyltransferase is selected from DNMT3A and DNMT3L and a combination thereof, and the zinc finger protein-based transcription factor is KRAB.

17. The composition of any of the preceding claims, wherein the at least one DNA binding protein is a Cas9, dCas9, Cpf1, a zinc finger nuclease (ZNF), a transcription activator-like effector nuclease (TALEN), a homing endonuclease, a dCas9-FokI nuclease or a MegaTal nuclease.

18. The composition of claim 17, wherein the at least one DNA binding protein is dCas9.

19. The composition of claim 18, wherein the dCas9 comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, a Streptococcus thermophilus (e.g., strain LMD-9) dCas9.

20. The composition of claim 18, wherein the dCas9 comprises the amino acid sequence of SEQ ID NO: 1.

21. The composition of any of the preceding claims, wherein the fusion molecule comprises the at least one modulator of gene expression fused to the C-terminus, the N-terminus, or both, of the at least one DNA binding protein.

22. The composition of claim 21, wherein the at least one modulator of gene expression is fused directly to the at least one DNA binding protein.

23. The composition of claim 21, wherein the at least one modulator of gene expression is fused indirectly with the at least one DNA binding protein via a non-modulator, a second modulator, or a linker.

24. The composition of any of claims 21-23, wherein the fusion molecule comprises a dCas9 fused with a KRAB on the C-terminal end and a DNMT3A and a DNMT3L on the N-terminal end.

25. The composition of claim 24, wherein the fusion molecule comprises the amino acid sequence of SEQ ID NO: 28.

26. The composition of any of the preceding claims, wherein the fusion molecule further comprises at least one nuclear localization sequence.

27. The composition of claim 26, wherein the at least one nuclear localization sequence is directly or indirectly fused to the C-terminus, the N-terminus or both of the at least one DNA binding protein.

28. The composition of any of the preceding claims, wherein the nucleic acid sequence encoding the fusion molecule is a deoxyribonucleic acid (DNA) or a messenger ribonucleic acid (mRNA).

29. The composition of any of the preceding claims, further comprising at least one single guide RNA (sgRNA) that is complementary to a target DNA sequence near the VEGF gene and/or within a VEGF regulatory element.

30. The composition of claim 29, wherein the target DNA sequence is located within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream or downstream of the transcription start site of the VEGF (e. g. VEGF-A) gene.

31. The composition of claim 29 or 30, wherein the sgRNA comprises the nucleic acid sequence of SEQ ID NOs: 29-58 and 60-84.

32. The composition of any of the preceding claims, wherein the fusion molecule is packaged in a liposome or a lipid nanoparticle.

33. The composition of any of claims 29-31, wherein the fusion molecule and the sgRNA are packaged in a liposome or a lipid nanoparticle.

34. The composition of claim 33, wherein the fusion molecule and the sgRNA are packaged in the same liposome or lipid nanoparticle, or in different liposomes or lipid nanoparticles.

35. The composition of any one of claims 32-34, wherein the liposome or the lipid nanoparticle comprises of ionizable lipids (20%-70%, molar ratio), PEGylated lipids (0%-30%, molar ratio), supporting lipids (30%-50%, molar ratio), and cholesterol (10%-50%, molar ratio).

36. The composition of claim 35, wherein the ionizable lipid is selected from a group consisting of pH-responsive ionizable lipids, thermal-responsive ionizable lipids and light-responsive ionizable lipids.

37. The composition of any of claims 1-31, wherein the fusion molecule is packaged in an AAV vector.

38. The composition of any one of claims 29-31, wherein the fusion molecule and the sgRNA are packaged in an AAV vector.

39. The composition of claim 38, wherein the fusion molecule and the sgRNA are packaged in the same AAV vector or in different AAV vectors.

40. The composition of any of the preceding claims, wherein the composition is a pharmaceutical composition comprising a pharmaceutically acceptable carrier.

41. A sgRNA, comprising a sequence complementary to a target DNA sequence located near the VEGF gene and/or within a VEGF regulatory element, optionally located within 500 bp upstream to 500 bp downstream of the transcription start site of VEGF gene.

42. The sgRNA of claim 41, comprising the nucleic acid sequence of any one of SEQ ID NOs: 29-58 and 60-84, optionally further comprising a tracr sequence as set forth in SEQ ID No: 59.

43. The sgRNA of claim 41 or 42, wherein the VEGF gene is VEGF-A gene from a mammalian animal, such as human, monkey, mouse, rat, and rabbit.

44. A nucleic acid molecule encoding the sgRNA of any one of claims 41 to 43.

45. A composition comprising:
(a) a fusion molecule, comprising a least one DNA binding protein and at least one modulator of gene expression, or a nucleic acid sequence encoding the fusion molecule; and
(b) a guiding molecule, comprising the sgRNA of any one of claims 41-43 and a protein binding sequence that is capable of binding to the at least one DNA binding protein, or a nucleic acid sequence encoding the guiding molecule;
wherein the at least one modulator of gene expression provides a modification of at least one nucleotide near the VEGF gene and/or within a VEGF regulatory element.

46. A method for reducing or eliminating the expression of a VEGF gene product in a cell comprising the step of introducing the composition of any one of claims 1-40 and 44 into the cell, thereby reducing or eliminating the expression of the VEGF gene product in the cell.

47. An in vivo method of reducing or eliminating the expression of a VEGF gene product in a subject, comprising the step of introducing the composition of any one of claims 1-40 and 44 to a cell of the subject, thereby reducing or eliminating the expression of the VEGF gene product in the subject.

48. A method for treating or alleviating a symptom of a VEGF related disorder in a subject, comprising the step of introducing an effective amount of the composition of any one of claims 1-40 and 44 to a cell of the subject.

49. The method of any one of claims 47-48, wherein the subject is a mammalian, such as human, monkey, mouse, rat, rabbit, pig, horse, cat and dog.

50. The method of any one of claims 48-49, wherein the VEGF related disorder is associated with angiogenesis.

51. The method of claim 50, wherein the VEGF related disorder is neovascular disorder, such as an ocular neovascular disorder, including age related macular degeneration (AMD).

52. The method of any one of claims 46-51, wherein the cell is a retinal cell, retinal pigment epithelial (RPE) cell or choroidal cell.

53. The method of any one of claims 47-52, wherein the fusion molecule is delivered to the subject by local injection such as intraocular injection and intravitreal injection.

54. The composition of any one of claims 1-40 and 44 for use in treating or alleviating a symptom of a VEGF related disorder in a subject.

55. The composition for use according to claim 54, wherein the VEGF related disorder is neovascular disorder, such as an ocular neovascular disorder, including Age related macular degeneration (AMD).

56. Use of the composition of any one of claims 1-40 and 44 in the manufacture of a medicament for treating or alleviating a symptom of a VEGF related disorder in a subject.

57. A kit, comprising a container that comprises the composition of any one of claims 1-40 and 44.
